SEQUENCE LISTING (58 sequences)

SEQ ID NO: 1 - protein, 326 aa, synthetic construct
MRTEAQVPALQPPEPGLEGAMGHRTLVLPWVLLTLCVTAGTPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLAGILGVSGVLLFGCVYLLHLLRRHKHRPAPRLQPSRTSPQAPRARAWAPSQASQAALHVPYATINTSCRPATLDTAHPHGGPSWWASLPTHAAHRPQGPAAWASTPIPARGSFVSVENGLYAQAGERPPHTGPGLTLFPDPRGPRAMEGPLGVR

SEQ ID NO: 2 - protein, 479 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPRASPRDVGPLVWGAVGGTLLVLLLLAGGSLAFILLRVRRRRKSPGGAGGGASGDGGFYDPKAQVLGNGDPVFWTPVVPGPMEPDGKDEEEEEEEEKAEKGLMLPPPPALEDDMESQLDGSLISRRAVYV

SEQ ID NO: 3 - protein, 518 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSY

SEQ ID NO: 4 - protein, 120 aa, synthetic construct
QVQLVQSGAEVKKPGASVKVSCKASGYTFTDYNINWVRQAPGQGLEWMGYIYPYIGGSGYAQKFQGRVTMTRDTSTSTVYMELSSLRSEDTAVYYCAREDKTARNAMDYWGQGTLVTVSS

SEQ ID NO: 5 - protein, 8 aa, synthetic construct
GYTFTDYN

SEQ ID NO: 6 - protein, 8 aa, synthetic construct
IYPYIGGS

SEQ ID NO: 7 - protein, 13 aa, synthetic construct
AREDKTARNAMDY

SEQ ID NO: 8 - protein, 447 aa, synthetic construct
QVQLVQSGAEVKKPGASVKVSCKASGYTFTDYNINWVRQAPGQGLEWMGYIYPYIGGSGYAQKFQGRVTMTRDTSTSTVYMELSSLRSEDTAVYYCAREDKTARNAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 9 - protein, 107 aa, synthetic construct
DIQMTQSPSSLSASVGDRVTITCRVSENIYSNLAWYQQKPGKAPKLLIYEATNLAEGVPSRFSGSGSGTDFTLTISSLQPEDFATYYCQHFWGTPYTFGQGTKLEIK

SEQ ID NO: 10 - protein, 6 aa, synthetic construct
ENIYSN

SEQ ID NO: 11 - skipped

SEQ ID NO: 12 - protein, 9 aa, synthetic construct
QHFWGTPYT

SEQ ID NO: 13 - protein, 214 aa, synthetic construct
DIQMTQSPSSLSASVGDRVTITCRVSENIYSNLAWYQQKPGKAPKLLIYEATNLAEGVPSRFSGSGSGTDFTLTISSLQPEDFATYYCQHFWGTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 119 aa, synthetic construct
QVQLVQSGAEVKKPGASVKVSCKASGYAFTNYLIEWVRQAPGQGLEWIGVINPGSGGIYYAQKFQGRVTMTADTSTSTVYMELSSLRSEDTAVYYCARSETHDTWFAYWGQGTLVTVSS

SEQ ID NO: 15 - protein, 8 aa, synthetic construct
GYAFTNYL

SEQ ID NO: 16 - protein, 8 aa, synthetic construct
INPGSGGI

SEQ ID NO: 17 - protein, 12 aa, synthetic construct
ARSETHDTWFAY

SEQ ID NO: 18 - protein, 446 aa, synthetic construct
QVQLVQSGAEVKKPGASVKVSCKASGYAFTNYLIEWVRQAPGQGLEWIGVINPGSGGIYYAQKFQGRVTMTADTSTSTVYMELSSLRSEDTAVYYCARSETHDTWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - protein, 107 aa, synthetic construct
DIQMTQSPSSLSASVGDRVTITCKASQSVRIAVAWFQQKPGKAPKALIYLASTRHTGVPSRFSGSGSGTDFTLTISSVQPEDFATYYCLQHWNYPYTFGQGTKLEIK

SEQ ID NO: 20 - protein, 6 aa, synthetic construct
QSVRIA

SEQ ID NO: 21 - skipped

SEQ ID NO: 22 - protein, 9 aa, synthetic construct
LQHWNYPYT

SEQ ID NO: 23 - protein, 214 aa, synthetic construct
DIQMTQSPSSLSASVGDRVTITCKASQSVRIAVAWFQQKPGKAPKALIYLASTRHTGVPSRFSGSGSGTDFTLTISSVQPEDFATYYCLQHWNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 121 aa, synthetic construct
EVQLVETGGGLIQPGRSLRLSCAASGFTFSSYAMHWVRQAPGKGLEWVAVISYDGTPVYYADSVKGRFTISRDNSKNTLYLQMNSLRAEDTAVYYCARDPLPLHYYGMDVWGQGTTVTVSS

SEQ ID NO: 25 - protein, 8 aa, synthetic construct
GFTFSSYA

SEQ ID NO: 26 - protein, 8 aa, synthetic construct
ISYDGTPV

SEQ ID NO: 27 - protein, 14 aa, synthetic construct
ARDPLPLHYYGMDV

SEQ ID NO: 28 - protein, 448 aa, synthetic construct
EVQLVETGGGLIQPGRSLRLSCAASGFTFSSYAMHWVRQAPGKGLEWVAVISYDGTPVYYADSVKGRFTISRDNSKNTLYLQMNSLRAEDTAVYYCARDPLPLHYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 29 - protein, 111 aa, synthetic construct
QSALTQPRSASGNPGQRVTISCSGSSSNMGRRPVNWYQQIPGTAPKLLIYSQNQRPSGVPDRFSGSQSGTSASLTISGLQSEDEAEYFCAVWDGDRRSLQLGGGTQLAVLG

SEQ ID NO: 30 - protein, 8 aa, synthetic construct
SSNMGRRP

SEQ ID NO: 31 - skipped

SEQ ID NO: 32 - protein, 11 aa, synthetic construct
AVWDGDRRSLQ

SEQ ID NO: 33 - protein, 216 aa, synthetic construct
QSALTQPRSASGNPGQRVTISCSGSSSNMGRRPVNWYQQIPGTAPKLLIYSQNQRPSGVPDRFSGSQSGTSASLTISGLQSEDEAEYFCAVWDGDRRSLQLGGGTQLAVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 121 aa, synthetic construct
EVQLVETGGGLIQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSS

SEQ ID NO: 35 - protein, 8 aa, synthetic construct
GFTFSSYA

SEQ ID NO: 36 - protein, 8 aa, synthetic construct
ISYAGEVK

SEQ ID NO: 37 - protein, 14 aa, synthetic construct
ARDPLPLHYYGMDV

SEQ ID NO: 38 - protein, 448 aa, synthetic construct
EVQLVETGGGLIQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 39 - protein, 111 aa, synthetic construct
QSALTQPRSASGNPGQRVTISCSGSSSNMGRRPVNWYQQIPGTAPKLLIYSQNQRPSGVPDRFSGSQSGTSASLTISGLQSEDEAEYFCAVWDDIGRVLQLGGGTQLAVLG

SEQ ID NO: 40 - protein, 8 aa, synthetic construct
SSNMGRRP

SEQ ID NO: 41 - skipped

SEQ ID NO: 42 - protein, 11 aa, synthetic construct
AVWDDIGRVLQ

SEQ ID NO: 43 - protein, 216 aa, synthetic construct
QSALTQPRSASGNPGQRVTISCSGSSSNMGRRPVNWYQQIPGTAPKLLIYSQNQRPSGVPDRFSGSQSGTSASLTISGLQSEDEAEYFCAVWDDIGRVLQLGGGTQLAVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 44 - protein, 120 aa, synthetic construct
EVQLVLSGGGFVQPGGSLKLSCAASGFTFSSYAMSWVRQNPERRLVWVATITGGGRNTYYPDSVKGRFTISRDNAKNTLYLQMSSLRSEDTAMYYCTRQGYDGYTWFAYWGQGTLVTVSS

SEQ ID NO: 45 - protein, 8 aa, synthetic construct
GFTFSSYA

SEQ ID NO: 46 - protein, 8 aa, synthetic construct
ITGGGRNT

SEQ ID NO: 47 - protein, 13 aa, synthetic construct
TRQGYDGYTWFAY

SEQ ID NO: 48 - protein, 447 aa, synthetic construct
EVQLVLSGGGFVQPGGSLKLSCAASGFTFSSYAMSWVRQNPERRLVWVATITGGGRNTYYPDSVKGRFTISRDNAKNTLYLQMSSLRSEDTAMYYCTRQGYDGYTWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 49 - protein, 112 aa, synthetic construct
DIVLTQSPTSLAVSLGQRATISCRASESVDNSGISFMNWFQQKPGQPPKLLIYAASNPGSGVPARFSGSGSGTDFSLNIHPMEEDDTAMYFCQQSKEVPWTFGGGTELEIKR

SEQ ID NO: 50 - protein, 10 aa, synthetic construct
ESVDNSGISF

SEQ ID NO: 51 - skipped

SEQ ID NO: 52 - protein, 9 aa, synthetic construct
QQSKEVPWT

SEQ ID NO: 53 - protein, 218 aa, synthetic construct
DIVLTQSPTSLAVSLGQRATISCRASESVDNSGISFMNWFQQKPGQPPKLLIYAASNPGSGVPARFSGSGSGTDFSLNIHPMEEDDTAMYFCQQSKEVPWTFGGGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 330 aa, Homo sapiens [sequence omitted]

SEQ ID NO: 55 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 56 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 57 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 300 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEG

(19) United States
(12) Patent Application Publication
(10) Pub. No.: US 2026/0000760 A1
(43) Pub. Date: Jan. 1, 2026

(54) TRIPLE COMBINATION CANCER THERAPY WITH ANTI-PVRIG ANTIBODIES, ANTI-TIGIT ANTIBODIES, AND PEMBROLIZUMAB

(71) Applicant: COMPUGEN LTD., Holon (IL)

(72) Inventors: Adeboye Henry Adewoye, MD, San Francisco, CA (US); Pierre Ferre, Toulouse (FR); Inbal Barbiro, Rehovot (IL); Eran Ophir, Even Yehuda (IL); Gad S. Cojocaru, Ramat-HaSharon (IL); Michelle Mahler, Efrat (IL); Lihi Zimmer-Strulov, Yoqneam Illit (IL)

(21) Appl. No.: 19/205,900

(22) Filed: May 12, 2025

Related U.S. Application Data
(60) Provisional application No. 63/648,608, filed on May 16, 2024.
(60) Provisional application No. 63/664,151, filed on Jun. 25, 2024.
(60) Provisional application No. 63/688,200, filed on Aug. 28, 2024.
(60) Provisional application No. 63/709,972, filed on Oct. 21, 2024.
(60) Provisional application No. 63/716,692, filed on Nov. 5, 2024.
(60) Provisional application No. 63/774,038, filed on Mar. 18, 2025.

(51) Int. Cl. A61K 39/395; A61K 39/00; A61K 47/02; A61K 47/18; A61K 47/22; A61K 47/26; A61P 35/00; A61P 35/04; C07K 16/28; G01N 33/574

(52) U.S. Cl. CPC A61K 39/39558; A61K 39/39591; A61K 47/02; A61K 47/183; A61K 47/22; A61K 47/26; A61P 35/00; A61P 35/04; C07K 16/2803; G01N 33/57484; A61K 2039/507; A61K 2039/545; G01N 2333/5412

(57) ABSTRACT

The present invention is directed to methods of maintenance treatment for platinum sensitive ovarian cancer, wherein the methods comprise administering an anti-PVRIG antibody, optionally in combination with anti-TIGIT antibodies and/or anti-PD-1 antibodies, including the use of stable liquid pharmaceutical formulations thereof, in particular of the anti-PVRIG antibodies. Also provided are combination therapies for the treatment of cancer refractory or resistant to at least two or more prior therapeutic treatments, wherein the treatment comprises anti-PVRIG antibodies, anti-TIGIT antibodies, and pembrolizumab, including the use of stable liquid pharmaceutical formulations thereof, in particular of the anti-PVRIG antibodies.

### CROSS-REFERENCE TO RELATED APPLICATIONS

This application claims priority to U.S. Provisional Patent Application No. 63/648,608, filed on May 16, 2024, U.S. Provisional Patent Application No. 63/664,151, filed Jun. 25, 2024, U.S. Provisional Patent Application No. 63/688,200, filed Aug. 28, 2024, U.S. Provisional Patent Application No. 63/709,972, filed Oct. 21, 2024, U.S. Provisional Patent Application No. 63/716,692, filed Nov. 5, 2024, and U.S. Provisional Patent Application No. 63/774,038, filed Mar. 18, 2025, which are hereby incorporated by reference in their entireties.

### SEQUENCE LISTING

The instant application contains a Sequence Listing which has been submitted electronically in XML format and is hereby incorporated by reference in its entirety. Said XML copy, created on Sep. 17, 2025, is named 114386-5031-US_SL.xml and is 54,000 bytes in size.

### BACKGROUND OF THE INVENTION

Both positive and negative costimulatory signals play critical roles in the regulation of cell-mediated immune responses, and molecules that mediate these signals have proven to be effective targets for immunomodulation. Based on this knowledge, several therapeutic approaches that involve targeting of costimulatory molecules have been developed, and were shown to be useful for prevention and treatment of cancer by turning on, or preventing the turning off, of immune responses in cancer patients or subjects and for prevention and treatment of autoimmune diseases and inflammatory diseases, as well as rejection of allogenic transplantation, each by turning off uncontrolled immune responses, or by induction of “off signal” by negative costimulation (or coinhibition) in subjects with these pathological conditions.

With regard to treatment therapies, a particular pathway and target of interest is PVRIG. PVRIG is a transmembrane domain protein of 326 amino acids in length, with a signal peptide (spanning from amino acid 1 to 40), an extracellular domain (spanning from amino acid 41 to 171), a transmembrane domain (spanning from amino acid 172 to 190) and a cytoplasmic domain (spanning from amino acid 191 to 326). The full length human PVRIG protein is shown in FIG. 1. There are two methionines that can be start codons, but the mature proteins are identical.

The PVRIG proteins contain an immunoglobulin (Ig) domain within the extracellular domain, which is a PVR-like Ig fold domain. The PVR-like Ig fold domain may be responsible for functional counterpart binding, by analogy to the other B7 family members. The PVR-like Ig fold domain of the extracellular domain includes one disulfide bond formed between intra domain cysteine residues, as is typical for this fold and may be important for structure-function. These cysteines are located at residues 22 and 93 (or 94). In one embodiment, there is provided a soluble fragment of PVRIG that can be used in testing of PVRIG antibodies. Included within the definition of PVRIG proteins are PVRIG ECD fragments, including know ECD fragments such as those described in U.S. Pat. No. 9,714,289.

Anti-PVRIG antibodies (including antigen-binding fragments) that both bind to PVRIG and prevent activation by PVRL2 (e.g., most commonly by blocking the interaction of PVRIG and PVLR2), are used to enhance T cell and/or NK cell activation and be used in treating diseases such as cancer and pathogen infection. As such, formulations for administering such antibodies are needed.

TIGIT is a coinhibitory receptor that is highly expressed on effector & regulatory (Treg) CD4+ T cells, effector CD8+ T cells, and NK cells. TIGIT has been shown to attenuate immune response by (1) direct signaling, (2) inducing ligand signaling, and (3) competition with and disruption of signaling by the costimulatory receptor CD226 (also known as DNAM-1). TIGIT signaling has been the most well-studied in NK cells, where it has been demonstrated that engagement with its cognate ligand, poliovirus receptor (PVR, also known as CD155) directly suppresses NK cell cytotoxicity through its cytoplasmic ITIM domain. Knockout of the TIGIT gene or antibody blockade of the TIGIT/PVR interaction has shown to enhance NK cell killing in vitro, as well as to exacerbate autoimmune diseases in vivo. In addition to its direct effects on T- and NK cells, TIGIT can induce PVR-mediated signaling in dendritic or tumor cells, leading to the increase in production of anti-inflammatory cytokines such as IL10. In T-cells TIGIT can also inhibit lymphocyte responses by disrupting homodimerization of the costimulatory receptor CD226, and by competing with it for binding to PVR.

TIGIT is highly expressed on lymphocytes, including Tumor Infiltrating Lymphocytes (TILs) and Tregs, that infiltrate different types of tumors. PVR is also broadly expressed in tumors, suggesting that the TIGIT-PVR signaling axis may be a dominant immune escape mechanism for cancer. Notably, TIGIT expression is tightly correlated with the expression of another important coinhibitory receptor, PD1. TIGIT and PD1 are co-expressed on the TILs of numerous human and murine tumors. Unlike TIGIT and CTLA4, PD1 inhibition of T cell responses does not involve competition for ligand binding with a costimulatory receptor.

The immune checkpoint, poliovirus receptor related immunoglobulin domain containing (PVRIG, also known as CD112R) represents a new inhibitory receptor within the TIGIT family of receptors. PVRIG binds with high affinity to its cognate ligand, poliovirus receptor-related 2 (PVRL2, also known as CD112 or nectin-2) to deliver an inhibitory signal through its ITIM motif within T and NK cells. The affinity of TIGIT to PVR and of PVRIG to PVRL2 is higher than the affinity of CD226 to either PVR or PVRL2, suggesting TIGIT and PVRIG can outcompete PVR and PVRL2 from CD226 and providing an indirect mechanism by which TIGIT and PVRIG can reduce lymphocyte function. Thus, two receptors with the same family, TIGIT and PVRIG, deliver inhibitory signals to dampen T and NK cell responses.

Accordingly, antibodies to both TIGIT and PVRIG are useful for triple therapy combinations pembrolizumab and meet the unmet need of providing triple combination therapies for use in treating patients or subjects refractory or resistant to multiple lines of treatment.

### BRIEF SUMMARY OF THE INVENTION

Accordingly, it is an object of the invention to provide triple combination therapies for methods of treatment or for use in treatment, wherein the triple combination includes stable liquid pharmaceutical formulations comprising anti-PVRIG antibodies, anti-TIGIT antibodies, and pembrolizumab.

The present invention provides a method of treatment for cancer in a subject refractory or resistant to at least two or more prior therapeutic treatments, the method comprising administering an anti-TIGIT antibody, and anti-PVRIG antibody, and pembrolizumab, wherein:
a) the anti-PVRIG antibody comprises:
   i) a heavy chain variable domain comprising the vhCDR1, vhCDR2, and vhCDR3 from the heavy chain variable domain of CHA.7.518.1.H4(S241P) (SEQ ID NO:4), and
   ii) a light chain variable domain comprising the vlCDR1, vlCDR2, and vlCDR3 from the light chain variable domain of CHA.7.518.1.H4(S241P) (SEQ ID NO:9); or
   i) a heavy chain variable domain comprising the vhCDR1, vhCDR2, and vhCDR3 from the heavy chain variable domain of CHA.7.538.1.2.H4(S241P) (SEQ ID NO:14), and
   ii) a light chain variable domain comprising the vlCDR1, vlCDR2, and vlCDR3 from the light chain variable domain of CHA.7.538.1.2.H4(S241P) (SEQ ID NO:19); and
b) the anti-TIGIT antibody comprises:
   i) a heavy chain variable domain comprising the vhCDR1, vhCDR2, and vhCDR3 from the heavy chain variable domain of CPA.9.083.H4(S241P) (SEQ ID NO:24), and
   ii) a light chain variable domain comprising the vlCDR1, vlCDR2, and vlCDR3 from the light chain variable domain of CPA.9.083.H4(S241P) (SEQ ID NO:29); or
   i) a heavy chain variable domain comprising the vhCDR1, vhCDR2, and vhCDR3 from the heavy chain variable domain of CPA.9.086.H4(S241P) (SEQ ID NO:34), and
   ii) a light chain variable domain comprising the vlCDR1, vlCDR2, and vlCDR3 from the light chain variable domain of CPA.9.086.H4(S241P) (SEQ ID NO:39).

In some embodiments, the anti-PVRIG antibody comprises:
i) a heavy chain variable domain comprising a vhCDR1 comprising SEQ ID NO: 5, a vhCDR2 comprising SEQ ID NO: 6, a vhCDR3 comprising SEQ ID NO: 7; and
ii) a light chain variable domain comprising a vlCDR1 comprising SEQ ID NO: 10, a vlCDR2 comprising SEQ ID NO: 11, a vlCDR3 comprising SEQ ID NO: 12.

In some embodiments, the anti-PVRIG antibody comprises:
i) a heavy chain variable domain comprising SEQ ID NO:4, and
ii) a light chain variable domain comprising SEQ ID NO:9.

In some embodiments, the anti-PVRIG antibody comprises:
i) a heavy chain comprising a vhCDR1 comprising SEQ ID NO: 5, a vhCDR2 comprising SEQ ID NO: 6, a vhCDR3 comprising SEQ ID NO: 7; and
ii) a light chain comprising a vlCDR1 comprising SEQ ID NO: 10, a vlCDR2 comprising SEQ ID NO: 11, a vlCDR3 comprising SEQ ID NO: 12;
wherein the heavy chain has at least 90% identity to SEQ ID NO: 8, and the light chain has at least 90% identity to SEQ ID NO: 13.

In some embodiments, the anti-PVRIG antibody comprises:
i) a heavy chain variable domain comprising a vhCDR1 comprising SEQ ID NO: 15, a vhCDR2 comprising SEQ ID NO: 16, a vhCDR3 comprising SEQ ID NO: 17; and
ii) a light chain variable domain comprising a vlCDR1 comprising SEQ ID NO: 20, a vlCDR2 comprising SEQ ID NO: 21, a vlCDR3 comprising SEQ ID NO: 22.

In some embodiments, the anti-PVRIG antibody comprises:
i) a heavy chain variable domain comprising SEQ ID NO:14, and
ii) a light chain variable domain comprising SEQ ID NO: 19.

In some embodiments, the anti-PVRIG antibody comprises:
i) a heavy chain comprising a vhCDR1 comprising SEQ ID NO: 15, a vhCDR2 comprising SEQ ID NO: 16, a vhCDR3 comprising SEQ ID NO: 17; and
ii) a light chain comprising a vlCDR1 comprising SEQ ID NO: 20, a vlCDR2 comprising SEQ ID NO: 21, a vlCDR3 comprising SEQ ID NO: 22;
wherein the heavy chain has at least 90% identity to SEQ ID NO: 18, and the light chain has at least 90% identity to SEQ ID NO: 23.

In some embodiments, the anti-TIGIT antibody comprises:
i) a heavy chain variable domain comprising a vhCDR1 comprising SEQ ID NO: 25, a vhCDR2 comprising SEQ ID NO: 26, a vhCDR3 comprising SEQ ID NO: 27; and
ii) a light chain variable domain comprising a vlCDR1 comprising SEQ ID NO: 30, a vlCDR2 comprising SEQ ID NO: 31, a vlCDR3 comprising SEQ ID NO: 32.

In some embodiments, the anti-TIGIT antibody comprises:
i) a heavy chain variable domain comprising SEQ ID NO:24, and
ii) a light chain variable domain comprising SEQ ID NO:29.

In some embodiments, the anti-TIGIT antibody comprises:
i) a heavy chain comprising a vhCDR1 comprising SEQ ID NO: 25, a vhCDR2 comprising SEQ ID NO: 26, a vhCDR3 comprising SEQ ID NO: 27; and
ii) a light chain comprising a vlCDR1 comprising SEQ ID NO: 30, a vlCDR2 comprising SEQ ID NO: 31, a vlCDR3 comprising SEQ ID NO: 32;
wherein the heavy chain has at least 90% identity to SEQ ID NO: 28, and the light chain has at least 90% identity to SEQ ID NO: 33.

In some embodiments, the anti-TIGIT antibody comprises:
i) a heavy chain variable domain comprising a vhCDR1 comprising SEQ ID NO: 35, a vhCDR2 comprising SEQ ID NO: 36, a vhCDR3 comprising SEQ ID NO: 37; and
ii) a light chain variable domain comprising a vlCDR1 comprising SEQ ID NO: 40, a vlCDR2 comprising SEQ ID NO: 41, a vlCDR3 comprising SEQ ID NO: 42.

In some embodiments, the anti-TIGIT antibody comprises:
i) a heavy chain variable domain comprising SEQ ID NO:34, and
ii) a light chain variable domain comprising SEQ ID NO:39.

In some embodiments, the anti-TIGIT antibody comprises:
i) a heavy chain comprising a vhCDR1 comprising SEQ ID NO: 35, a vhCDR2 comprising SEQ ID NO: 36, a vhCDR3 comprising SEQ ID NO: 37; and
ii) a light chain comprising a vlCDR1 comprising SEQ ID NO: 40, a vlCDR2 comprising SEQ ID NO: 41, a vlCDR3 comprising SEQ ID NO: 42;
wherein the heavy chain has at least 90% identity to SEQ ID NO: 38, and the light chain has at least 90% identity to SEQ ID NO: 43.

In some embodiments, pembrolizumab comprises:
i) a heavy chain variable domain comprising a vhCDR1 comprising SEQ ID NO: 45, a vhCDR2 comprising SEQ ID NO: 46, a vhCDR3 comprising SEQ ID NO: 47; and
ii) a light chain variable domain comprising a vlCDR1 comprising SEQ ID NO: 50, a vlCDR2 comprising SEQ ID NO: 51, a vlCDR3 comprising SEQ ID NO: 52.

In some embodiments, pembrolizumab comprises:
i) a heavy chain variable domain comprising SEQ ID NO: 44, and
ii) a light chain variable domain comprising SEQ ID NO: 49.

In some embodiments, pembrolizumab comprises:
i) a heavy chain comprising a vhCDR1 comprising SEQ ID NO: 45, a vhCDR2 comprising SEQ ID NO: 46, a vhCDR3 comprising SEQ ID NO: 47; and
ii) a light chain comprising a vlCDR1 comprising SEQ ID NO: 50, a vlCDR2 comprising SEQ ID NO: 51, a vlCDR3 comprising SEQ ID NO: 52;
wherein the heavy chain has at least 90% identity to SEQ ID NO: 48, and the light chain has at least 90% identity to SEQ ID NO: 53.

In some embodiments, the anti-PVRIG antibody is administered as a stable liquid pharmaceutical formulation and, wherein the stable liquid pharmaceutical formulation of the anti-PVRIG antibody comprises:
(a) from 10 mM to 100 mM histidine;
(b) from 30 mM to 100 mM NaCl;
(c) from 20 mM to 150 mM L-Arginine; and
(d) from 0.005% to 0.1% w/v polysorbate 80,
wherein the formulation has a pH from 5.5 to 7.0.

In some embodiments, the anti-PVRIG antibody and/or the anti-TIGIT antibody comprises a CH1-hinge-CH2-CH3 sequence of IgG4 (SEQ ID NO:57 or SEQ ID NO:58), wherein the hinge region optionally comprises mutations.

In some embodiments, the anti-PVRIG antibody and/or the anti-TIGIT antibody comprises the CH1-hinge-CH2-CH3 region from IgG1, IgG2, IgG3, or IgG4, wherein the hinge region optionally comprises mutations. In some embodiments, the IgG1 comprises SEQ ID NO:54. In some embodiments, the IgG2 comprises SEQ ID NO:55. In some embodiments, the IgG3 comprises SEQ ID NO:56. In some embodiments, the IgG4 comprises SEQ ID NO:57. In some embodiments, the IgG4 comprises SEQ ID NO:58.

In some embodiments, the anti-PVRIG heavy chain variable domain is from the heavy chain of CHA.7.518.1.H4(S241P) (SEQ ID NO:8) and the anti-PVRIG light chain variable domain is from the light chain of CHA.7.518.1.H4(S241P) (SEQ ID NO:13).

In some embodiments, the anti-PVRIG heavy chain variable domain is from the heavy chain of CHA.7.538.1.2.H4(S241P) (SEQ ID NO:18) and the anti-PVRIG light chain variable domain is from the light chain of CHA.7.538.1.2.H4(S241P) (SEQ ID NO:23).

In some embodiments, the anti-PVRIG antibody comprises a CL region of human kappa 2 light chain.

In some embodiments, the pharmaceutical formulation comprises from 10 mM to 80 mM histidine, from 15 mM to 70 mM histidine, from 20 mM to 60 mM histidine, from 20 mM to 50 mM histidine, or from 20 mM to 30 mM histidine.

In some embodiments, the pharmaceutical formulation comprises about 25 mM histidine.

In some embodiments, the pharmaceutical formulation comprises from 30 mM to 100 mM NaCl, from 30 mM to 90 mM NaCl, from 40 mM to 80 mM NaCl, from 30 mM to 70 mM histidine, or from 45 mM to 70 mM NaCl.

In some embodiments, the pharmaceutical formulation comprises about 60 mM NaCl.

In some embodiments, the pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine.

In some embodiments, the pharmaceutical formulation comprises about 100 mM L-arginine.

In some embodiments, pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, or from 0.009% to 0.05% w/v polysorbate 80.

In some embodiments, the pharmaceutical formulation comprises about 0.01% polysorbate 80.

In some embodiments, the pH is from 6 to 7.0. In some embodiments, the pH is from 6.3 to 6.8. In some embodiments, the pH is 6.5+/−0.2.

In some embodiments, the anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL.

In some embodiments, the anti-PVRIG antibody formulation is stable at −20° C. for at least 1 month, 3 months, 6 months, 9 months, 12 months, 18 months, 24 months, 30 months, 36 months, 42 months or 48 months.

In some embodiments, the anti-PVRIG antibody formulation is stable at 2° C. to 8° C. for at least 1 week, 2 weeks, 3 weeks, 4 weeks, 5 weeks, 6 weeks, 7 weeks, 8 weeks, 9 weeks, or 10 weeks, 1 month, 3 months, 6 months, 9 months, 12 months, 18 months, 24 months, 30 months, 36 months, 42 months or 48 months.

In some embodiments, the anti-PVRIG antibody formulation is stable at about 20° C. to 25° C. for at least 1 week, 2 weeks, 3 weeks, 4 weeks, 5 weeks, or 6 weeks, 1 months, 3 months, 6 months, or 9 months, 12 months, 18 months, or 36 months.

In some embodiments, the anti-PVRIG antibody formulation is stable at 35° C. to 40° C. for at least 1 week, 2 weeks, 3 weeks, 4 weeks, 5 weeks, 6 weeks, 7 weeks, or 8 weeks.

In some embodiments, the anti-PVRIG antibody comprises:
a) a heavy chain comprising:
   i) a VH-CH1-hinge-CH2-CH3, wherein the VH is from CHA.7.518.1.H4(S241P) (SEQ ID NO:4) and wherein the CH1-hinge-CH2-CH3 region is from IgG4; and
b) a light chain comprising:
   i) a VL-CL, wherein the VL from CHA.7.518.1.H4(S241P) (SEQ ID NO:9) and wherein the CL region is from human kappa 2 light chain; or
a) a heavy chain comprising:
   i) a VH-CH1-hinge-CH2-CH3, wherein the VH is from CHA.7.538.1.H4(S241P) (SEQ ID NO:14) and wherein the CH1-hinge-CH2-CH3 region is from IgG4; and
b) a light chain comprising:
   i) a VL-CL, wherein the VL from CHA.7.538.1.H4(S241P) (SEQ ID NO:19) and wherein the CL region is from human kappa 2 light chain.

In some embodiments, the hinge region optionally comprises mutations.

In some embodiments, the anti-PVRIG antibody formulation comprises:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13); or
i) a heavy chain comprising the heavy chain from CHA.7.538.1.2.H4(S241P) (SEQ ID NO:18); and
ii) a light chain comprising the light chain from CHA.7.538.1.2.H4(S241P) (SEQ ID NO:23).

In some embodiments, the anti-PVRIG antibody formulation comprises:
(a) an anti-PVRIG antibody, wherein the anti-PVRIG antibody comprises:
   i) a heavy chain variable domain comprising the vhCDR1, vhCDR2, and vhCDR3 from the heavy chain variable domain of CHA.7.518.1.H4(S241P) (SEQ ID NO:4), and
   ii) a light chain variable domain comprising the vlCDR1, vlCDR2, and vlCDR3 from the light chain variable domain of CHA.7.518.1.H4(S241P) (SEQ ID NO:9); or
   an anti-PVRIG antibody, wherein the anti-PVRIG antibody comprises:
   i) a heavy chain comprising the heavy chain variable domain from CHA.7.538.1.2.H4(S241P) (SEQ ID NO:14); and
   ii) a light chain comprising the light chain variable domain from CHA.7.538.1.2.H4(S241P) (SEQ ID NO:19);
(b) about 25 mM histidine;
(c) about 60 mM NaCl;
(d) about 100 mM L-Arginine; and
(e) about 0.01% % w/v polysorbate 80,
wherein the formulation has a pH from 6.5+/−0.2.

In some embodiments, the anti-PVRIG antibody formulation comprising:
(a) an anti-PVRIG antibody, wherein the anti-PVRIG antibody comprises:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13); or
   an anti-PVRIG antibody, wherein the anti-PVRIG antibody comprises:
   i) a heavy chain comprising the heavy chain from CHA.7.538.1.2.H4(S241P) (SEQ ID NO:18); and
   ii) a light chain comprising the light chain from CHA.7.538.1.2.H4(S241P) (SEQ ID NO:23);
(b) about 25 mM histidine;
(c) about 60 mM NaCl;
(d) about 100 mM L-Arginine; and
(e) about 0.01% % w/v polysorbate 80,
wherein the formulation has a pH from 6.5+/−0.2.

In some embodiments, the anti-TIGIT antibody comprises:
a) a heavy chain comprising VH-CH1-hinge-CH2-CH3; and
b) a light chain comprising VL-VC, wherein VC is either kappa or lambda.

In some embodiments, the sequence of the CH1-hinge-CH2-CH3 is selected from human IgG1, IgG2 and IgG4, and variants thereof.

In some embodiments, the hinge region optionally comprises mutations.

In some embodiments, the anti-TIGIT heavy chain variable domain is from the heavy chain of CPA.9.083.H4(S241P) (SEQ ID NO:28) and the anti-TIGIT light chain variable domain is from the light chain of CPA.9.083.H4(S241P) (SEQ ID NO:33).

In some embodiments, the anti-TIGIT heavy chain variable domain is from the heavy chain of CPA.9.086.H4(S241P) (SEQ ID NO:38) and the anti-TIGIT light chain variable domain is from the light chain of CPA.9.086.H4(S241P) (SEQ ID NO:43).

In some embodiments, the anti-TIGIT antibody comprises:
i) a heavy chain comprising the heavy chain from CPA.9.083.H4(S241P) (SEQ ID NO:28); and
ii) a light chain comprising the light chain from CPA.9.083.H4(S241P) (SEQ ID NO:33); or
i) a heavy chain comprising the heavy chain from CPA.9.086.H4(S241P) (SEQ ID NO:38); and
ii) a light chain comprising the light chain from CPA.9.086.H4(S241P) (SEQ ID NO:43).

In some embodiments, the anti-PVRIG antibody, anti-TIGIT antibody and pembrolizumab combination is administered every 1 week, 2 weeks, 3 weeks, or 4 weeks.

In some embodiments, the anti-PVRIG antibody, anti-TIGIT antibody and pembrolizumab combination is administered every 3 weeks.

In some embodiments, the anti-PVRIG antibody is administered at a dosage of about 0.01 mg/kg to about 20 mg/kg of the anti-PVRIG antibody, about 0.01 mg/kg to about 15 mg/kg of the anti-PVRIG antibody or about 0.01 mg/kg to about 10 mg/kg of the anti-PVRIG antibody.

In some embodiments, the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg of the anti-PVRIG antibody.

In some embodiments, the anti-PVRIG antibody is administered at a concentration of about 15 mg/kg or about 20 mg/kg.

In some embodiments, the anti-PVRIG antibody is administered at a concentration of about 20 mg/kg every 3 weeks.

In some embodiments, the anti-PVRIG antibody is administered at a concentration of about 15 mg/kg every 3 weeks.

In some embodiments, the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the anti-TIGIT antibody is administered about 10 mg/kg every 3 weeks.

In some embodiments, the anti-TIGIT antibody is administered about 3 mg/kg every 3 weeks.

In some embodiments, pembrolizumab is administered as a dosage of about 2 mg/kg or 10 mg/kg. In some embodiments, pembrolizumab is administered as a dosage of about 2 mg/kg. In some embodiments, pembrolizumab is administered as a dosage of about 10 mg/kg.

In some embodiments, pembrolizumab is administered as a dosage of about 2 mg/kg every 3 weeks. In some embodiments, pembrolizumab is administered as a dosage of about 10 mg/kg every 3 weeks. In some embodiments, pembrolizumab is administered as a dosage of about 200 mg every 3 weeks. In some embodiments, pembrolizumab is administered as a dosage of about 400 mg every 6 weeks.

In some embodiments, the anti-PVRIG antibody, anti-TIGIT antibody, and pembrolizumab are administered in any order.

In some embodiments, the anti-PVRIG antibody, anti-TIGIT antibody, and pembrolizumab are administered simultaneously.

In some embodiments, the anti-PVRIG antibody, anti-TIGIT antibody, and pembrolizumab are administered sequentially.

In some embodiments, the order of administration is one of:
i) anti-PVRIG antibody, anti-TIGIT antibody, and pembrolizumab;
ii) anti-TIGIT antibody, anti-PVRIG antibody, and pembrolizumab;
iii) pembrolizumab, anti-PVRIG antibody, and anti-TIGIT antibody;
iv) pembrolizumab, anti-TIGIT antibody, and anti-PVRIG antibody;
v) anti-PVRIG antibody, pembrolizumab, and anti-TIGIT antibody; or
vi) anti-TIGIT antibody, pembrolizumab, and anti-PVRIG antibody.

In some embodiments, pembrolizumab is administered over about 10 minutes, over about 15 minutes, over about 20 minutes, over about 25 minutes, over about 30 minutes, over about 35 minutes, or over about 40 minutes.

In some embodiments, the pembrolizumab is administered over about 10 minutes to about 40 minutes, over about 15 minutes to about 40 minutes, over about 20 minutes to about 40 minutes, or about 30 minutes +/−10 minutes.

In some embodiments, the level of serum IFNγ in the subject is increased by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1100%, 1200%, 1300%, 1400%, 1500% or more, relative to untreated subjects. In some embodiments, the level of serum IFNγ in the subject is increased by at least 200%, 300%, 400%, or more, relative to untreated subjects. In some embodiments, the level of serum IFNγ in the subject is increased by at least 1000%, 1100%, 1200%, 1300%, 1400%, 1500% or more, relative to untreated subjects. In some embodiments, the level of serum IFNγ in the subject is increased by at least 1200%, or more, relative to untreated subjects.

In some embodiments, the subject exhibits an increased level of serum IFNγ of at least 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 110 pg/mL, 120 pg/mL, 130 pg/mL, 140 pg/mL, 150 pg/mL, 160 pg/mL, 170 pg/mL, 180 pg/mL, 190 pg/mL, 200 pg/mL, 210 pg/mL, 220 pg/mL, 230 pg/mL, 240 pg/mL, 250 pg/mL, 260 pg/mL, 270 pg/mL, 280 pg/mL, 290 pg/mL, 300 pg/mL, 310 pg/mL, 320 pg/mL, 330 pg/mL, 340 pg/mL, 350 pg/mL, 360 pg/mL, 370 pg/mL, 380 pg/mL, 390 pg/mL, 400 pg/mL, 410 pg/mL, 420 pg/mL, 430 pg/mL, 440 pg/mL, 450 pg/mL, 460 pg/mL, 470 pg/mL, 480 pg/mL, 490 pg/mL, 500 pg/mL, or more. In some embodiments, the subject exhibits an increased level of serum IFNγ of at least 20 pg/mL, 30 pg/mL, 40 pg/mL, or more. In some embodiments, the subject exhibits an increased level of serum IFNγ of at least 40 pg/mL, 50 pg/mL, 60 pg/mL, 70 pg/mL, 80 pg/mL or more. In some embodiments, the subject exhibits an increased level of serum IFNγ of at least 70 pg/mL, or more. In some embodiments, the subject exhibits an increased level of serum IFNγ of at least 80 pg/mL, or more.

In some embodiments, the serum level of IL-6 in the subject is decreased by at least about 10%, 20%, 30%, 40%, 50%, 60%, 70%, 80%, or 90% relative to untreated subjects.

In some embodiments, the subject exhibits a decreased and/or lower baseline serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally the subject exhibits a decreased and/or lower baseline serum level of IL-6 of no more than about 1 pg/mL, 2 pg/mL, or 3 pg/mL.

In some embodiments, the subject exhibits a decreased and/or lower baseline serum level of IL-6 for at least about 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, or 40 weeks.

In some embodiments, the serum level of IL-8 in the subject is decreased and/or lower by at least about 10%, 20%, 30%, 40%, 50%, 60%, 70%, 80%, or 90% relative to untreated subjects.

In some embodiments, the subject exhibits a decreased and/or lower baseline serum level of IL-8 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally a decreased and/or lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL.

In some embodiments, the subject exhibits a decreased and/or lower baseline serum level of IL-8 for at least about 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, or 40 weeks.

In some embodiments, the cancer is selected from the group consisting of prostate cancer, liver cancer (HCC), rectal cancer, colorectal cancer (CRC), colorectal cancer MSS (MSS-CRC; microsatellite stable colorectal carcinoma/carcinoma), CRC (MSS unknown), metastatic MSS-CRC, refractory metastatic MSS-CRC, MSS-CRC with liver metastasis, ovarian cancer (including ovarian carcinoma), primary peritoneal ovarian carcinoma, advanced epithelial ovarian cancer, advanced epithelial ovarian carcinoma, platinum resistant ovarian cancer, platinum sensitive epithelial ovarian cancer, platinum sensitive high grade ovarian cancer, platinum sensitive fallopian tube cancer, platinum sensitive peritoneal cancer, platinum sensitive primary peritoneal cancer, platinum sensitive ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1 >=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

In some embodiments, the anti-PVRIG antibody, anti-TIGIT antibody, and pembrolizumab combination can find use in a method of treating cancer in a subject.

In some embodiments, the invention provides for the use of the anti-PVRIG antibody, anti-TIGIT antibody, and pembrolizumab in the manufacture of a medicament for the treatment of cancer in a subject, wherein the anti-PVRIG antibody is formulated as the stable liquid pharmaceutical formulation as described herein.

In some embodiments of the use in a method of treating cancer, the cancer is selected from the group consisting of prostate cancer, liver cancer (HCC), rectal cancer, colorectal cancer (CRC), colorectal cancer MSS (MSS-CRC; microsatellite stable colorectal carcinoma/carcinoma), CRC (MSS unknown), metastatic MSS-CRC, refractory metastatic MSS-CRC, MSS-CRC with liver metastasis, ovarian cancer (including ovarian carcinoma), primary peritoneal ovarian carcinoma, advanced epithelial ovarian cancer, advanced epithelial ovarian carcinoma, platinum resistant ovarian cancer, platinum sensitive epithelial ovarian cancer, platinum sensitive high grade ovarian cancer, platinum sensitive fallopian tube cancer, platinum sensitive peritoneal cancer, platinum sensitive primary peritoneal cancer, platinum sensitive ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1 >=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

In some embodiments of the formulation, method, or use of the invention as described herein, the subject is refractory or resistant to treatment with at least two or more prior therapeutic treatments, wherein the prior therapeutic treatments include one or more of fluroropyrimidines (or fluoropyrimidines), irinotecan, oxaliplatin, mirvetuximab soravtansine-gynx, anti-PD-1, anti-PD-L1, anti-PD-L2, anti-CD96 antibody, anti-OX-40 antibody, anti-CD137 antibody, anti-LAG3, anti-TIM3, and/or anti-CTLA4 antibody therapies.

In some embodiments of the formulation, method, or use of the invention as described herein, the subject is refractory or resistant to treatment with at least two or more prior therapeutic treatments, wherein the two or more prior therapeutic treatments include fluroropyrimidines (or fluoropyrimidines), irinotecan, oxaliplatin, and/or mirvetuximab soravtansine-gynx.

In some embodiments of the formulation, method, or use of the invention as described herein, the subject is refractory or resistant to treatment with no more than three prior therapeutic treatments.

In some embodiments of the formulation, method, or use of the invention as described herein, the subject is refractory or resistant to treatment with at least three or more prior therapeutic treatments.

In some embodiments of the formulation, method, or use of the invention as described herein, the subject is refractory or resistant to treatment with at least three or more prior therapeutic treatments, where in the three prior therapeutic treatments include fluroropyrimidines (or fluoropyrimidines), irinotecan, oxaliplatin, and/or mirvetuximab soravtansine-gynx.

In some embodiments of the formulation, method, or use of the invention as described herein, the subject has not had prior treatment with an anti-PD-1, anti-PD-L1, or anti-PD-L2-directed therapy.

In some embodiments of the formulation, method, or use of the invention as described herein, the subject has not had prior treatment with an immune checkpoint inhibitor, including anti-PD-1, anti-PD-L1, anti-PD-L2, anti-CD96 antibody, anti-OX-40 antibody, anti-CD137 antibody, anti-LAG3, anti-TIM3, and/or anti-CTLA4 antibody therapies.

In another aspect, the present invention provides herein a method for determining a cancer patient population for treatment with an anti-PVRIG antibody, an anti-TIGIT antibody, and an anti-PD-1 antibody, the method comprising:
(a) detecting the presence of IL-6 in the serum from the cancer patient,
(b) quantitating the measurement of the level of IL-6;
(c) treating the cancer patient with an anti-PVRIG antibody, an anti-TIGIT antibody, and an anti-PD-1 antibody when IL-6 is present at a decreased and/or lower baseline serum level as compared to a control or a patient that is not treated with the anti-PVRIG antibody, and the anti-TIGIT antibody, and the anti-PD-1 antibody.

In another aspect, the present invention provides herein a method for predicting or determining the efficacy of treatment with an anti-PVRIG antibody, an anti-TIGIT antibody, and an anti-PD-1 antibody in a subject, the method comprising:
(a) measuring the serum level of IL-6 in the subject;
(b) quantitating the measurement of the serum level of IL-6;
(c) correlating the serum level of IL-6 with the efficacy of treatment, wherein IL-6 at a decreased and/or lower baseline serum level as compared to a control or a subject not treated with the anti-PVRIG antibody, the anti-TIGIT antibody, and the anti-PD-1 antibody, is indicative of treatment efficacy.

In some embodiments, the method further comprises:
(d) treating the cancer patient with an anti-PVRIG antibody, an anti-TIGIT antibody, and an anti-PD-1 antibody when IL-6 is at a decreased and/or lower baseline serum level as compared to a control or a subject not treated with the anti-PVRIG antibody, the anti-TIGIT antibody, and the anti-PD-1 antibody.

In some embodiments, a decreased and/or lower baseline serum level of IL-6 is one of the following:
(a) at least about 10%, 20%, 30%, 40%, 50%, 60%, 70%, 80%, or 90% relative to a control or a subject not treated with the anti-PVRIG antibody, the anti-TIGIT antibody, and the anti-PD-1 antibody; and
(b) no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally no more than about 1 pg/mL, 2 pg/mL, or 3 pg/mL.

In some embodiments, a decreased and/or lower baseline serum level of IL-6 of at least about 10%, 20%, 30%, 40%, 50%, 60%, 70%, 80%, or 90% relative to a control or a subject not treated with the anti-PVRIG antibody, the anti-TIGIT antibody, and the anti-PD-1 antibody is indicative of efficacy of treatment with the anti-PVRIG antibody, the anti-TIGIT antibody, and the anti-PD-1 antibody.

In some embodiments, a cancer patient having a serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally having a serum level of IL-6 of no more than about 1 pg/mL, 2 pg/mL, or 3 pg/mL, exhibits a higher probability to respond to the treatment with the anti-PVRIG antibody, the anti-TIGIT antibody, and the anti-PD-1 antibody as compared to a control or a patient not treated with the anti-PVRIG antibody, the anti-TIGIT antibody, and the anti-PD-1 antibody.

In some embodiments, the anti-PVRIG antibody is the anti-PVRIG antibody according to any of the embodiments provided herein.

In some embodiments, the anti-PVRIG antibody is administered as a stable liquid pharmaceutical formulation according to any of the embodiments provided herein.

In some embodiments, the anti-TIGIT antibody is the anti-TIGIT antibody according to any of the embodiments provided herein.

In some embodiments, the anti-PD-1 antibody is selected from the group consisting of: pembrolizumab, nivolumab, cemiplimab, SHR-1210 (CTR20160175 and CTR20170090), SHR-1210 (CTR20170299 and CTR20170322), JS-001 (CTR20160274), IBI308 (CTR20160735), BGB-A317 (CTR20160872), AGEN2033w, AGEN2034w, AGEN2046w, AGEN2047w, AGEN2047w-N297A, AGEN2047w-S267E/L328F, AGEN2047w-S239D/A330L/I332E, nivolumab BMS-936558/MDX-1106/ONO-4538, AMP224, CT-011, and MK-3475. In some embodiments, the anti-PD-1 antibody is pembrolizumab.

In another aspect, the present invention provides herein a method for determining a cancer patient population for treatment with an anti-PVRIG antibody, an anti-TIGIT antibody, and an anti-PD-1 antibody, the method comprising:
(a) detecting the presence of IL-8 in the serum from the cancer patient,
(b) quantitating the measurement of the level of IL-8;
(c) treating the cancer patient with an anti-PVRIG antibody, an anti-TIGIT antibody, and an anti-PD-1 antibody when IL-8 is present at a decreased and/or lower baseline serum level as compared to a control or a patient that is not treated with the anti-PVRIG antibody, and the anti-TIGIT antibody, and the anti-PD-1 antibody.

In another aspect, the present invention provides herein a method for determining a cancer patient population for treatment with an anti-PVRIG antibody, an anti-TIGIT antibody, and an anti-PD-1 antibody, the method comprising:
(a) detecting the presence of IL-8 in the serum from the cancer patient,
(b) quantitating the measurement of the level of IL-8;
(c) treating the cancer patient with an anti-PVRIG antibody, an anti-TIGIT antibody, and an anti-PD-1 antibody when IL-8 is present at a decreased and/or lower baseline serum level as compared to a control or a patient that is not treated with the anti-PVRIG antibody, and the anti-TIGIT antibody, and the anti-PD-1 antibody.

In another aspect, the present invention provides herein a method for predicting or determining the efficacy of treatment with an anti-PVRIG antibody, an anti-TIGIT antibody, and an anti-PD-1 antibody in a subject, the method comprising:
(a) measuring the serum level of IL-8 in the subject;
(b) quantitating the measurement of the serum level of IL-8;
(c) correlating the serum level of IL-8 with the efficacy of treatment, wherein IL-8 at a decreased and/or lower baseline serum level as compared to a control or a subject not treated with the anti-PVRIG antibody, the anti-TIGIT antibody, and the anti-PD-1 antibody, is indicative of treatment efficacy.

In some embodiments, the method further comprises:
(d) treating the cancer patient with an anti-PVRIG antibody, an anti-TIGIT antibody, and an anti-PD-1 antibody when IL-8 is at a decreased and/or lower baseline serum level as compared to a control or a subject not treated with the anti-PVRIG antibody, the anti-TIGIT antibody, and the anti-PD-1 antibody.

In some embodiments, a decreased and/or lower baseline serum level of IL-8 is one of the following:
(a) at least about 10%, 20%, 30%, 40%, 50%, 60%, 70%, 80%, or 90% relative to a control or a subject not treated with the anti-PVRIG antibody, the anti-TIGIT antibody, and the anti-PD-1 antibody; and
(b) no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL.

In some embodiments, a decreased and/or lower baseline serum level of IL-8 of at least about 10%, 20%, 30%, 40%, 50%, 60%, 70%, 80%, or 90% relative to a control or a subject not treated with the anti-PVRIG antibody, the anti-TIGIT antibody, and the anti-PD-1 antibody is indicative of efficacy of treatment with the anti-PVRIG antibody, the anti-TIGIT antibody, and the anti-PD-1 antibody.

In some embodiments, a cancer patient having a serum level of IL-8 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally having a serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, exhibits a higher probability to respond to the treatment with the anti-PVRIG antibody, the anti-TIGIT antibody, and the anti-PD-1 antibody as compared to a control or a patient not treated with the anti-PVRIG antibody, the anti-TIGIT antibody, and the anti-PD-1 antibody.

In some embodiments, the anti-PVRIG antibody is the anti-PVRIG antibody according to any of the embodiments provided herein.

In some embodiments, the anti-PVRIG antibody is administered as a stable liquid pharmaceutical formulation according to any of the embodiments provided herein.

In some embodiments, the anti-TIGIT antibody is the anti-TIGIT antibody according to any of the embodiments provided herein.

In some embodiments, the anti-PD-1 antibody is selected from the group consisting of: pembrolizumab, nivolumab, cemiplimab, SHR-1210 (CTR20160175 and CTR20170090), SHR-1210 (CTR20170299 and CTR20170322), JS-001 (CTR20160274), IBI308 (CTR20160735), BGB-A317 (CTR20160872), AGEN2033w, AGEN2034w, AGEN2046w, AGEN2047w, AGEN2047w-N297A, AGEN2047w-S267E/L328F, AGEN2047w-S239D/A330L/I332E, nivolumab BMS-936558/MDX-1106/ONO-4538, AMP224, CT-011, and MK-3475. In some embodiments, the anti-PD-1 antibody is pembrolizumab.

In some embodiments, the anti-PD-1 antibody is selected from the group consisting of: pembrolizumab, cemiplimab, SHR-1210 (CTR20160175 and CTR20170090), SHR-1210 (CTR20170299 and CTR20170322), JS-001 (CTR20160274), IBI308 (CTR20160735), BGB-A317 (CTR20160872), AGEN2033w, AGEN2034w, AGEN2046w, AGEN2047w, AGEN2047w-N297A, AGEN2047w-S267E/L328F, AGEN2047w-S239D/A330L/I332E, AMP224, CT-011, and MK-3475.

In some embodiments, the anti-PD-1 antibody is pembrolizumab.

In some embodiments, the anti-PD-1 antibody is nivolumab.

In some embodiments, the anti-PD-1 antibody is cemiplimab.

In some embodiments, the serum level of IL-6, IL-8, and/or both are measured. In some embodiments, the serum level of IL-6, IL-8, and/or both are each measured using a human pro-inflammatory Panel 1 cytokine 10-plex assay kit. In some embodiments, the kit is the kit commercially available from Meso Scael Diagnostics (see, the Word Wide Web at mesoscale.com/products/v-plex-proinflammatory-panel-1-human-kit-k15049d/.

In another aspect, the present invention provides herein a method of maintenance treatment for platinum sensitive ovarian cancer, platinum sensitive epithelial ovarian cancer, platinum sensitive high grade ovarian cancer, platinum sensitive fallopian tube cancer, platinum sensitive peritoneal cancer, or platinum sensitive primary peritoneal cancer, the method comprising administering an anti-PVRIG antibody to a subject in need thereof, wherein:
the anti-PVRIG antibody comprises:
   a heavy chain variable domain comprising the vhCDR1, vhCDR2, and vhCDR3 from the heavy chain variable domain of CHA.7.518.1.H4(S241P) (SEQ ID NO:4), and
   a light chain variable domain comprising the vlCDR1, vlCDR2, and vlCDR3 from the light chain variable domain of CHA.7.518.1.H4(S241P) (SEQ ID NO:9); or
   a heavy chain variable domain comprising the vhCDR1, vhCDR2, and vhCDR3 from the heavy chain variable domain of CHA.7.538.1.2.H4(S241P) (SEQ ID NO:14), and
   a light chain variable domain comprising the vlCDR1, vlCDR2, and vlCDR3 from the light chain variable domain of CHA.7.538.1.2.H4(S241P) (SEQ ID NO:19).

In some embodiments, the subject has been previously treated with a platinum treatment and a standard of care maintenance therapeutic treatment.

In some embodiments, the subject has been previously treated with a platinum treatment and is ineligible and/or cannot tolerate a standard of care maintenance therapeutic treatment.

In some embodiments, the subject has not been previously treated with a platinum treatment for greater than or equal to about 6 months prior to relapse of the ovarian cancer.

In some embodiments, the subject is responsive to the previous platinum treatment.

In some embodiments, the prior maintenance therapeutic treatment comprises bevacizumab and/or a PARP inhibitor.

In some embodiments, the maintenance treatment further comprises administering to the subject in need thereof an anti-TIGIT antibody, an anti-PD-1 antibody, or a combination thereof. In some embodiments, the TIGIT antibody and/or the anti-PD-1 antibody are the TIGIT antibody and/or the anti-PD-1 antibody provided herein in the present disclosures.

In some embodiments, the maintenance treatment further comprises administering to the subject in need thereof bevacizumab and/or a PARP inhibitor.

In some embodiments, the anti-PVRIG antibody is administered as a stable liquid pharmaceutical formulation and, wherein the stable liquid pharmaceutical formulation of the anti-PVRIG antibody comprises:
a. from 10 mM to 100 mM histidine;
b. from 30 mM to 100 mM NaCl;
c. from 20 mM to 150 mM L-Arginine; and
d. from 0.005% to 0.1% w/v polysorbate 80,
wherein the formulation has a pH from 5.5 to 7.0.

In some embodiments, the anti-PVRIG antibody comprises a CH1-hinge-CH2-CH3 sequence of IgG4 (SEQ ID NO:57 or SEQ ID NO:58), wherein the hinge region optionally comprises mutations.

In some embodiments, the anti-PVRIG antibody comprises the CH1-hinge-CH2-CH3 region from IgG1, IgG2, IgG3, or IgG4, wherein the hinge region optionally comprises mutations, optionally wherein the IgG1 comprises SEQ ID NO:54, the IgG2 comprises SEQ ID NO:55, the IgG3 comprises SEQ ID NO:56, the IgG4 comprises SEQ ID NO:57, and/or the IgG4 comprises SEQ ID NO:58.

In some embodiments, the anti-PVRIG heavy chain variable domain is from the heavy chain of CHA.7.518.1.H4(S241P) (SEQ ID NO:8) and the anti-PVRIG light chain variable domain is from the light chain of CHA.7.518.1.H4(S241P) (SEQ ID NO:13).

In some embodiments, the anti-PVRIG heavy chain variable domain is from the heavy chain of CHA.7.538.1.2.H4(S241P) (SEQ ID NO:18) and the anti-PVRIG light chain variable domain is from the light chain of CHA.7.538.1.2.H4(S241P) (SEQ ID NO:23).

In some embodiments, the anti-PVRIG antibody comprises a CL region of human kappa 2 light chain.

In some embodiments, the pharmaceutical formulation comprises from 10 mM to 80 mM histidine, from 15 mM to 70 mM histidine, from 20 mM to 60 mM histidine, from 20 mM to 50 mM histidine, or from 20 mM to 30 mM histidine.

In some embodiments, the pharmaceutical formulation comprises about 25 mM histidine.

In some embodiments, the pharmaceutical formulation comprises from 30 mM to 100 mM NaCl, from 30 mM to 90 mM NaCl, from 40 mM to 80 mM NaCl, from 30 mM to 70 mM histidine, or from 45 mM to 70 mM NaCl.

In some embodiments, the pharmaceutical formulation comprises about 60 mM NaCl.

In some embodiments, the pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine.

In some embodiments, the pharmaceutical formulation comprises about 100 mM L-arginine.

In some embodiments, the pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, or from 0.009% to 0.05% w/v polysorbate 80.

In some embodiments, the pharmaceutical formulation comprises about 0.01% polysorbate 80.

In some embodiments, the pH is from 6 to 7.0. In some embodiments, the pH is from 6.3 to 6.8. In some embodiments, the pH is 6.5+/−0.2.

In some embodiments, the anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL.

In some embodiments, said formulation is stable at −20° C. for at least 1 month, 3 months, 6 months, 9 months, 12 months, 18 months, 24 months, 30 months, 36 months, 42 months or 48 months.

In some embodiments, the formulation is stable at 2° C. to 8° C. for at least 1 week, 2 weeks, 3 weeks, 4 weeks, 5 weeks, 6 weeks, 7 weeks, 8 weeks, 9 weeks, 10 weeks, 1 month, 3 months, 6 months, 9 months, 12 months, 18 months, 24 months, 30 months, 36 months, 42 months or 48 months.

In some embodiments, the formulation is stable at about 20° C. to 25° C. for at least 1 week, 2 weeks, 3 weeks, 4 weeks, 5 weeks, 6 weeks, 1 months, 3 months, 6 months, or 9 months, 12 months, 18 months, or 36 months.

In some embodiments, the formulation is stable at 35° C. to 40° C. for at least 1 week, 2 weeks, 3 weeks, 4 weeks, 5 weeks, 6 weeks, 7 weeks, or 8 weeks.

In some embodiments, the anti-PVRIG antibody comprises:
a heavy chain comprising:
   a VH-CH1-hinge-CH2-CH3, wherein the VH is from CHA.7.518.1.H4(S241P) (SEQ ID NO:4) and wherein the CH1-hinge-CH2-CH3 region is from IgG4; and
a light chain comprising:
   a VL-CL, wherein the VL from CHA.7.518.1.H4(S241P) (SEQ ID NO:9) and wherein the CL region is from human kappa 2 light chain; or
   a) a heavy chain comprising:
      i) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   b) a light chain comprising:
      i) a VL-CL, wherein the VL from CHA.7.538.1.H4(S241P) (SEQ ID NO:19) and wherein the CL region is from human kappa 2 light chain.

In some embodiments, the hinge region optionally comprises mutations.

In some embodiments, the anti-PVRIG antibody comprises:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13); or
i) a heavy chain comprising the heavy chain from CHA.7.538.1.2.H4(S241P) (SEQ ID NO:18); and
ii) a light chain comprising the light chain from CHA.7.538.1.2.H4(S241P) (SEQ ID NO:23).

In some embodiments, the anti-PVRIG antibody comprises:
i) a heavy chain variable domain comprising the vhCDR1, vhCDR2, and vhCDR3 from the heavy chain variable domain of CHA.7.518.1.H4(S241P) (SEQ ID NO:4), and
ii) a light chain variable domain comprising the vlCDR1, vlCDR2, and vlCDR3 from the light chain variable domain of CHA.7.518.1.H4(S241P) (SEQ ID NO:9); or
i) a heavy chain comprising the heavy chain from CHA.7.538.1.2.H4(S241P) (SEQ ID NO:14); and
ii) a light chain comprising the light chain from CHA.7.538.1.2.H4(S241P) (SEQ ID NO:19);
about 25 mM histidine;
about 60 mM NaCl;
about 100 mM L-Arginine; and
about 0.01% % w/v polysorbate 80,
   wherein the formulation has a pH from 6.5+/−0.2.

In some embodiments, the anti-PVRIG antibody comprises:
i) a heavy chain comprising the heavy chain from CHA.7.518.1.H4(S241P) (SEQ ID NO:8); and
ii) a light chain comprising the light chain from CHA.7.518.1.H4(S241P) (SEQ ID NO:13); or
i) a heavy chain comprising the heavy chain from CHA.7.538.1.2.H4(S241P) (SEQ ID NO:18); and
ii) a light chain comprising the light chain from CHA.7.538.1.2.H4(S241P) (SEQ ID NO:23);
about 25 mM histidine;
about 60 mM NaCl;
about 100 mM L-Arginine; and
about 0.01% % w/v polysorbate 80,
   wherein the formulation has a pH from 6.5+/−0.2.

In some embodiments, the sequence of the CH1-hinge-CH2-CH3 is selected from human IgG1, IgG2 and IgG4, and variants thereof, optionally wherein the IgG1 comprises SEQ ID NO:54, the IgG2 comprises SEQ ID NO:55, the IgG4 comprises SEQ ID NO:57, and/or the IgG4 comprises SEQ ID NO:58.

In some embodiments, the hinge region optionally comprises mutations.

In some embodiments, the anti-PVRIG antibody is administered every 1 week, 2 weeks, 3 weeks, or 4 weeks.

In some embodiments, the anti-PVRIG antibody is administered every 3 weeks.

In some embodiments, the anti-PVRIG antibody is administered at a dosage of about 0.01 mg/kg to about 20 mg/kg of the anti-PVRIG antibody, about 0.01 mg/kg to about 15 mg/kg of the anti-PVRIG antibody or about 0.01 mg/kg to about 10 mg/kg of the anti-PVRIG antibody.

In some embodiments, the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg of the anti-PVRIG antibody.

In some embodiments, the anti-PVRIG antibody is administered at a concentration of about 15 mg/kg or about 20 mg/kg.

In some embodiments, the anti-PVRIG antibody is administered at a concentration of about 20 mg/kg every 3 weeks.

In some embodiments, the anti-PVRIG antibody is administered at a concentration of about 15 mg/kg every 3 weeks.

In some embodiments, the anti-PVRIG antibody treatment described herein is for use in a method of treating cancer in a subject.

In another aspect, the present invention provides a use of the anti-PVRIG antibody in the manufacture of a medicament for the maintenance treatment of cancer in a subject, wherein the anti-PVRIG antibody is formulated as the stable liquid pharmaceutical formulation described herein.

In some embodiments, the formulation, method, or use described herein, comprises an anti-PVRIG antibody comprising:
i) a heavy chain variable domain comprising a vhCDR1 comprising SEQ ID NO: 5, a vhCDR2 comprising SEQ ID NO: 6, a vhCDR3 comprising SEQ ID NO: 7; and
ii) a light chain variable domain comprising a vlCDR1 comprising SEQ ID NO: 10, a vlCDR2 comprising SEQ ID NO: 11, a vlCDR3 comprising SEQ ID NO: 12.

In some embodiments, the formulation, method, or use described herein, comprises an anti-PVRIG antibody comprising:
i) a heavy chain variable domain comprising SEQ ID NO: 4, and
ii) a light chain variable domain comprising SEQ ID NO: 9.

In some embodiments, the formulation, method, or use described herein, comprises an anti-PVRIG antibody comprising:
i) a heavy chain comprising a vhCDR1 comprising SEQ ID NO: 5, a vhCDR2 comprising SEQ ID NO: 6, a vhCDR3 comprising SEQ ID NO: 7; and
ii) a light chain comprising a vlCDR1 comprising SEQ ID NO: 10, a vlCDR2 comprising SEQ ID NO: 11, a vlCDR3 comprising SEQ ID NO: 12;
wherein the heavy chain has at least 90% identity to SEQ ID NO: 8, and the light chain has at least 90% identity to SEQ ID NO: 13.

In some embodiments, the formulation, method, or use described herein, comprises an anti-PVRIG antibody comprising:
i) a heavy chain variable domain comprising a vhCDR1 comprising SEQ ID NO: 15, a vhCDR2 comprising SEQ ID NO: 16, a vhCDR3 comprising SEQ ID NO: 17; and
ii) a light chain variable domain comprising a vlCDR1 comprising SEQ ID NO: 20, a vlCDR2 comprising SEQ ID NO: 21, a vlCDR3 comprising SEQ ID NO: 22.

In some embodiments, the formulation, method, or use described herein, comprises an anti-PVRIG antibody comprising:
i) a heavy chain variable domain comprising SEQ ID NO: 14, and
ii) a light chain variable domain comprising SEQ ID NO: 19.

In some embodiments, the formulation, method, or use described herein, comprises an anti-PVRIG antibody comprising:
i) a heavy chain comprising a vhCDR1 comprising SEQ ID NO: 15, a vhCDR2 comprising SEQ ID NO: 16, a vhCDR3 comprising SEQ ID NO: 17; and
ii) a light chain comprising a vlCDR1 comprising SEQ ID NO: 20, a vlCDR2 comprising SEQ ID NO: 21, a vlCDR3 comprising SEQ ID NO: 22;
   wherein the heavy chain has at least 90% identity to SEQ ID NO: 18, and the light chain has at least 90% identity to SEQ ID NO: 23.

In some embodiments of the formulation, method, or use described herein, the platinum sensitive ovarian cancer is platinum sensitive epithelial ovarian cancer.

In some embodiments of the formulation, method, or use described herein, the platinum sensitive ovarian cancer is high grade ovarian cancer.

In some embodiments of the formulation, method, or use described herein, the platinum sensitive fallopian tube cancer is high grade fallopian tube cancer.

In some embodiments of the formulation, method, or use described herein, the platinum sensitive peritoneal cancer is high grade peritoneal cancer.

In some embodiments of the formulation, method, or use described herein, the platinum sensitive peritoneal cancer is primary peritoneal cancer.

### BRIEF DESCRIPTION OF THE DRAWINGS

FIG. 1 depicts the full-length sequence of human PVRIG.

FIG. 2 depicts the sequence of the human Poliovirus receptor-related 2 protein (PVLR2, also known as nectin-2, CD112 or herpesvirus entry mediator B, (HVEB)), the binding partner of PVRIG. PVLR2 is a human plasma membrane glycoprotein.

FIG. 3A-3B depicts the variable heavy and light chains as well as the vhCDR1, vhCDR2, vhCDR3, vlCDR1, vlCDR2 and vlCDR3 sequences of the anti-PVRIG antibodies of the invention CHA.7.518.1.H4(S241P) and CHA.7.538.1.2.H4(S241P).

FIG. 4A-4B depicts the variable heavy and light chains as well as the vhCDR1, vhCDR2, vhCDR3, vlCDR1, vlCDR2 and vlCDR3 sequences of the anti-TIGIT antibodies of the invention CPA.9.083.H4(S241P) and CPA.9.086.H4(S241P).

FIG. 5 depicts the variable heavy and light chains as well as the vhCDR1, vhCDR2, vhCDR3, vlCDR1, vlCDR2 and vlCDR3 sequences of pembrolizumab.

FIG. 6 depicts the sequences of human IgG1, IgG2, IgG3 and IgG4.

FIG. 7 depicts formulation parameters, including buffers and excipients for the CHA.7.518.1.H4(S241P) antibody formulation.

FIG. 8 depicts an exemplary study design overview.

FIG. 9 illustrates pharmacodynamic activation of the immune system with CHA.7.518.1.H4(S241P), CPA.9.086.H4(S241P), and Pembrolizumab. Peripheral blood was collected from 19 patients (17 CRC (gray) and 2 OVCA (black)) at baseline (C1D1), one day post treatment (C1D2), one week post treatment (C1D8) and before the start of consecutive drug administration (IV, Q3W; every 3 weeks, i.e., C2D1 reflects blood collected 3 weeks post-treatment and prior to the second cycle of treatment) of CHA.7.518.1.H4(S241P) (15 mg/kg), CPA.9.086.H4(S241P) (3 mg/kg) and Pembrolizumab (200 mg). Samples were assessed for serum IFNγ levels, measured using a pro-inflammatory human cytokine 10-plex assay kit and Meso Scale Discovery (MSD).

FIG. 10 illustrates DNAM-1 AXIS pathway and potential synergy in blocking PVRIG, TIGIT and PD-1 pathways.

FIG. 11 illustrates the study schema for the triplet combination expansion cohorts.

FIG. 12 illustrates baseline demographic and disease characteristics for participants with MSS-CRC.

FIG. 13 illustrates a disposition and exposure summary.

FIG. 14 illustrates a summary of TEAEs and serious TEAEs by preferred term and maximum grade.

FIG. 15 illustrates a summary of investigator-assessed tumor response (per RECIST v1.1).

FIGS. 16A-16C illustrate tumor responses in individual participants.

FIG. 17 illustrates imaging assessment for participant with partial response.

FIGS. 18A-18C illustrate pharmacodynamic activation of the immune system observed in tumor and serum after treatment in participants with MSS-CRC. (A) CD8+ T cell infiltration is increased post-treatment in tumor biopsies obtained between Cycle 2 day 1 and Cycle 3 Day 1 from individual participants treated with CHA.7.518.1.H4(S241P)+CPA.9.086.H4(S241P)+pembrolizumab (n=10) or CHA.7.518.1.H4(S241P)+nivolumab [symbol] (n=11)^{a}. (B) Serum IFN [symbol] levels^{b} in individual participants treated with CHA.7.518.1.H4(S241P)+CPA.9.086.H4(S241P)+pembrolizumab (N=20). (C) Mean (SE) serum IL-6 and IL-8 concentrations^{b} among participants with (n=3) and without (n=17) clinical benefit^{c} following treatment with CHA.7.518.1.H4(S241P)+CPA.9.086.H4(S241P)+pembrolizumab.

FIG. 19 illustrates serum IL-6 and IL-8 concentrations among participants, with metastatic MSS-CRC, with and without clinical benefit.

FIG. 20 illustrates serum IFN [symbol] levels in individual participants, with metastatic MSS-CRC, treated with CHA.7.518.1.H4(S241P)+CPA.9.086.H4(S241P)+pembrolizumab (N=20). Robust peripheral IFN [symbol] induction was observed after treatment with CHA.7.518.1.H4(S241P)+CPA.9.086.H4(S241P)+pembrolizumab. This increase was statistically significant at C1D2 (day 1 post-treatment) compared with C1D1 (pre-treatment). Serum cytokines were measured using a pro-inflammatory human cytokine 10-plex assay kit and Meso Scale Discovery.* p<0.05; **** p<0.0001.

FIG. 21 provides a waterfall plot, presenting target lesions best percent change from baseline, in participants with platinum resistant ovarian cancer (see, Example 8).

FIG. 22 provides tumor marker CA125 changes data in participants with platinum resistant ovarian cancer (see, Example 8).

FIG. 23 provides a Swimmer's plot in participants with platinum resistant ovarian cancer (see, Example 8).

[symbol]

FIG. 24 provides data regarding serum IFN levels in individual participants treated with CHA.7.518.1.H4(S241P)+CPA.9.086.H4(S241P)+pembrolizumab (N=23, with paired C1D1 and C1D2 samples). Robust peripheral IFNγ induction was observed after treatment with CHA.7.518.1.H4(S241P)+CPA.9.086.H4(S241P)+pembrolizumab. This increase was statistically significant at C1D2 (day 1 post-treatment) compared with C1D1 (pre-treatment). Serum cytokines were measured using a pro-inflammatory Panel 1 human cytokine 10-plex assay kit and Meso Scale Discovery. **** p<0.0001

FIG. 25 provides Mean (SE) serum IL-6 and IL-8 concentrations among participants with (n=8; CB) and without (n=16; NCB) clinical benefit following treatment with CHA.7.518.1.H4(S241P)+CPA.9.086.H4(S241P)+pembrolizumab. Significantly lower serum concentrations of IL-6 and IL-8 were observed among participants benefiting from treatment for more than 120 days, both pre-treatment and during treatment (CB=clinical benefit; NCB=no clinical benefit). Serum cytokines were measured using a pro-inflammatory Pane 1 human cytokine 10-plex assay kit and Meso Scale Discovery. Clinical benefit defined as on treatment for 120 days with a confirmed response of SD or better. * p<0.05; ** p<0.01, *** p<0.001

FIG. 26 provides baseline serum IL-6 concentrations among participants with (n=8; CB) and without (n=14; NCB) clinical benefit following treatment with CHA.7.518.1.H4(S241P)+CPA.9.086.H4(S241P)+pembrolizumab. (CB=clinical benefit; NCB=no clinical benefit)

FIG. 27 provides baseline serum IL-8 concentrations among participants with (n=8) and without (n=14) clinical benefit following treatment with CHA.7.518.1.H4(S241P)+CPA.9.086.H4(S241P)+pembrolizumab. (CB=clinical benefit; NCB=no clinical benefit)

FIG. 28 illustrates DNAM-1 AXIS Pathway: Potential Synergy in Blocking PVRIG, TIGIT, and PD-1 Pathways

FIG. 29 illustrates Clinical Trial Schema regarding Triplet Combination Expansion Cohorts.

FIG. 30 provides a summary of Baseline Demographic and Disease Characteristics.

FIG. 31 provides a Participant Disposition and Exposure Summary.

FIG. 32A-32C provides a summary of safety and adverse events. (A) Safety Summary. (B) TEAEs Reported in ≥3 Participants by Frequency and Severity. (C) Incidence of Grade ≥3 TEAEs.

FIG. 33 provides a summary of Investigator-assessed Tumor Response (RECIST v1.1) (EES).

FIG. 34A-34D illustrates Tumor Responses in Individual Participants (EES). (A): To account for the phenomenon of pseudoprogression or delayed response with ICI, continuation of trial treatment beyond RECIST-defined progression could continue at the discretion of the investigator. Participants were to be assessed again within 6-8 weeks from initial progression to confirm PD. Participants with confirmed PD but with clinical benefit as per investigator assessment could continue to receive trial treatment. (B) and (C) Change from baseline refers to the sum of diameters of target lesions according to RECIST 1.1. Two participants (#17 and #24) were not included, as target lesion response was assessed as inevaluable. (C) A dashed line means the participant is ongoing. (D) assessments on or after the end of treatment (EOT) were excluded. A dashed line means the participant is ongoing. BOR: Best overall response; C1D1: cycle 1 day 1; CR: complete response; EES: efficacy evaluable set; PD: progressive disease; PR: partial response; SD: stable disease.

FIG. 35 illustrates Clinical Vignette of a Participant with a Complete Response. Imaging Assessment of Participant with Complete Response.

FIG. 36A-36B illustrates Pharmacodynamic Activation of the Immune System. (A) A. Serum IFN- [symbol] Concentrations on C1D2 in Participants Treated with CHA.7.518.1.H4(S241P)+CPA.9.086.H4(S241P)+pembrolizumab (N=23). (B) Serum (mean [SE]) IL-6 and IL-8 Concentrations in Participants with vs without Clinical Benefit Before and After Treatment with CHA.7.518.1.H4(S241P)+CPA.9.086.H4(S241P)+pembrolizumab.

FIG. 37 illustrates the patient populations for platinum sensitive ovarian cancer and platinum resistant ovarian cancer.

FIG. 38 illustrates the mechanism of action of the anti-PVRIG therapy with CHA.7.518.1.H4(S241P).

FIG. 39 illustrates CHA.7.518.1.H4(S241P) single agent activity with partial response >18 month.

FIG. 40 illustrates the mechanism of action of the anti-PVRIG therapy with CHA.7.518.1.H4(S241P) in combination with therapies targeting other immune pathways.

FIG. 41 illustrates consistent efficacy & safety with CHA.7.518.1.H4(S241P) triple combination in platinum resistant ovarian cancer patients who have exhausted all treatment options.

FIG. 42 illustrates consistent durable responses with CHA.7.518.1.H4(S241P) triple combination demonstrated across studies in platinum resistant ovarian cancer patients who have exhausted all treatment options.

FIG. 43 illustrates consistent deep responses with CHA.7.518.1.H4(S241P) triple combination in platinum resistant ovarian cancer patients who have exhausted all treatment options.

FIG. 44 provides a clinical vignette—CHA.7.518.1.H4(S241P)+pembrolizumab+CPA.9.086.H4(S241P) complete response in PROC patient that has received 4 prior lines of therapy including chemotherapy, bevacizumab maintenance and an investigational agent (smac-mimetic) with SD best response prior to study entry.

FIG. 45 provides a clinical vignette—CHA.7.518.1.H4(S241P)+nivolumab partial response in nivolumab refractory PROC patient that has received 7 prior lines of therapy including progressed disease on nivolumab.

FIG. 46 illustrates that CHA.7.518.1.H4(S241P) drives clinical benefit and TME T cell clonal expansion independent of baseline inflammatory status.

FIG. 47 illustrates that CHA.7.518.1.H4(S241P) safety profile provides clinically relevant differentiation in earlier cancer settings.

FIG. 48 illustrates a CHA.7.518.1.H4(S241P) adaptive platform trial in relapsed platinum sensitive ovarian cancer in maintenance setting.

### DETAILED DESCRIPTION OF THE INVENTION

### I. Introduction

Cancer can be considered as an inability of the patient or subject (the terms patient and subject are generally used interchangeably) to recognize and eliminate cancerous cells. In many instances, these transformed (e.g., cancerous) cells counteract immunosurveillance. There are natural control mechanisms that limit T-cell activation in the body to prevent unrestrained T-cell activity, which can be exploited by cancerous cells to evade or suppress the immune response. Restoring the capacity of immune effector cells—especially T cells—to recognize and eliminate cancer is the goal of immunotherapy. The field of immuno-oncology, sometimes referred to as “immunotherapy” is rapidly evolving, with several recent approvals of T cell checkpoint inhibitory antibodies such as Yervoy, Keytruda and Opdivo. These antibodies are generally referred to as “checkpoint inhibitors” because they block normally negative regulators of T cell immunity. It is generally understood that a variety of immunomodulatory signals, both costimulatory and coinhibitory, can be used to orchestrate an optimal antigen-specific immune response. Generally, these antibodies bind to checkpoint inhibitor proteins such as CTLA4 and PD-1, which under normal circumstances prevent or suppress activation of cytotoxic T cells (CTLs). By inhibiting the checkpoint protein, for example through the use of antibodies that bind these proteins, an increased T cell response against tumors can be achieved. That is, these cancer checkpoint proteins suppress the immune response; when the proteins are blocked, for example using antibodies to the checkpoint protein, the immune system is activated, leading to immune stimulation, resulting in treatment of conditions such as cancer and infectious disease.

The present invention is directed to formulations comprising antibodies to human Poliovirus Receptor Related Immunoglobulin Domain Containing Protein, or “PVRIG”, sometimes also referred to herein as “PV protein”. PVRIG is expressed on the cell surface of NK and T-cells and shares several similarities to other known immune checkpoints for use in triple combination therapy with antibodies to TIGIT and in combination with pembrolizumab.

Accordingly, the present invention provides formulations comprising antibodies, including antigen binding domains, that bind to the human PVRIG and peptides thereof and methods of activating T cells and/or NK cells to treat diseases such as cancer and infectious diseases, and other conditions where increased immune activity results in treatment. In particular, the invention provides formulations comprising antibodies comprising heavy and light chains as well as the vhCDR1, vhCDR2, vhCDR3, vlCDR1, vlCDR2 and vlCDR3 sequences from CHA.7.518.1.H4(S241P) as well as heavy and light chains as well as the vhCDR1, vhCDR2, vhCDR3, vlCDR1, vlCDR2 and vlCDR3 sequences from CHA.7.538.1.H4(S241P).

Accordingly, the present invention also provides antibodies, including antigen binding domains, that bind to the human TIGIT protein and peptides thereof. In particular, the invention provides formulations comprising antibodies comprising heavy and light chains as well as the vhCDR1, vhCDR2, vhCDR3, vlCDR1, vlCDR2 and vlCDR3 sequences from CPA.9.083.H4(S241P) as well as heavy and light chains as well as the vhCDR1, vhCDR2, vhCDR3, vlCDR1, vlCDR2 and vlCDR3 sequences from CPA.9.086.H4(S241P).

### II. PVRIG Proteins

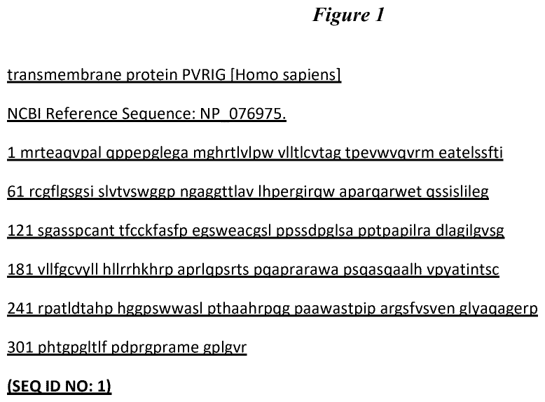

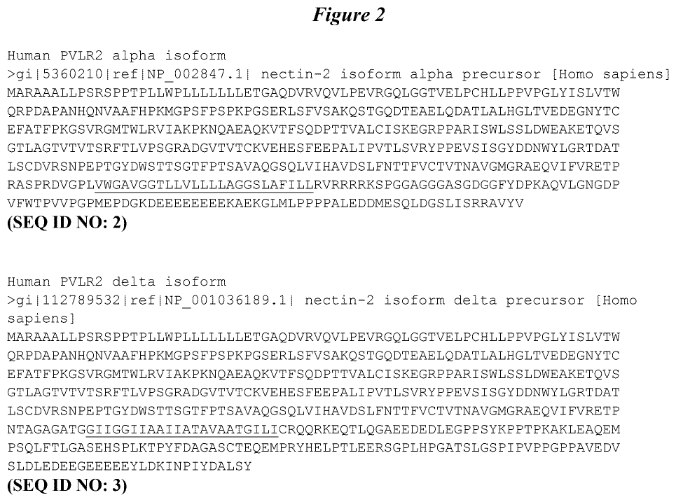

The present invention provides formulations comprising antibodies that specifically bind to PVRIG proteins. “Protein” in this context is used interchangeably with “polypeptide”, and includes peptides as well. The present invention provides antibodies that specifically bind to PVRIG proteins. PVRIG is a transmembrane domain protein of 326 amino acids in length, with a signal peptide (spanning from amino acid 1 to 40), an extracellular domain (spanning from amino acid 41 to 171), a transmembrane domain (spanning from amino acid 172 to 190) and a cytoplasmic domain (spanning from amino acid 191 to 326). The full length human PVRIG protein is shown in FIG. 1. There are two methionines that can be start codons, but the mature proteins are identical.

Accordingly, as used herein, the term “PVRIG” or “PVRIG protein” or “PVRIG polypeptide” may optionally include any such protein, or variants, conjugates, or fragments thereof, including but not limited to known or wild type PVRIG, as described herein, as well as any naturally occurring splice variants, amino acid variants or isoforms, and in particular the ECD fragment of PVRIG. The term “soluble” form of PVRIG is also used interchangeably with the terms “soluble ectodomain (ECD)” or “ectodomain” or “extracellular domain (ECD) as well as “fragments of PVRIG polypeptides”, which may refer broadly to one or more of the following optional polypeptides:

The PVRIG proteins contain an immunoglobulin (Ig) domain within the extracellular domain, which is a PVR-like Ig fold domain. The PVR-like Ig fold domain may be responsible for functional counterpart binding, by analogy to the other B7 family members. The PVR-like Ig fold domain of the extracellular domain includes one disulfide bond formed between intra domain cysteine residues, as is typical for this fold and may be important for structure-function. These cysteines are located at residues 22 and 93 (or 94). In one embodiment, there is provided a soluble fragment of PVRIG that can be used in testing of PVRIG antibodies. Included within the definition of PVRIG proteins are PVRIG ECD fragments, including know ECD fragments such as those described in U.S. Pat. No. 9,714,289, incorporate by reference herein in its entirety for all purposes.

As noted herein and more fully described below, the anti-PVRIG antibodies (including antigen-binding fragments) that both bind to PVRIG and prevent activation by PVRL2 (e.g. most commonly by blocking the interaction of PVRIG and PVLR2), are used to enhance T cell and/or NK cell activation and be used in treating diseases such as cancer and pathogen infection.

### III. TIGIT Proteins

The present invention provides antibodies that specifically bind to TIGIT proteins and prevent activation by its ligand protein, PVR, poliovirus receptor (aka, CD155) a human plasma membrane glycoprotein. TIGIT, or T cell immunoreceptor with Ig and ITIM domains, is a co-inhibitory receptor protein also known as WUCAM, Vstm3 or Vsig9. TIGIT has an immunoglobulin variable domain, a transmembrane domain, and an immunoreceptor tyrosine-based inhibitory motif (ITIM) and contains signature sequence elements of the PVR protein family. The extracellular domain (ECD) sequences of TIGIT and of PVR are shown in FIG. 1B. The antibodies of the invention are specific for the TIGIT ECD such that the binding of TIGIT and PVR is blocked

Accordingly, as used herein, the term “TIGIT” or “TIGIT protein” or “TIGIT polypeptide” may optionally include any such protein, or variants, conjugates, or fragments thereof, including but not limited to known or wild type TIGIT, as described herein, as well as any naturally occurring splice variants, amino acid variants or isoforms, and in particular the ECD fragment of TIGIT.

As noted herein and more fully described below, anti-TIGIT antibodies (including antigen-binding fragments) that both bind to TIGIT and prevent activation by PVR (e.g., most commonly by blocking the interaction of TIGIT and PVR), are used to enhance T cell and/or NK cell activation and be used in treating diseases such as cancer and pathogen infection.

### IV. Antibodies

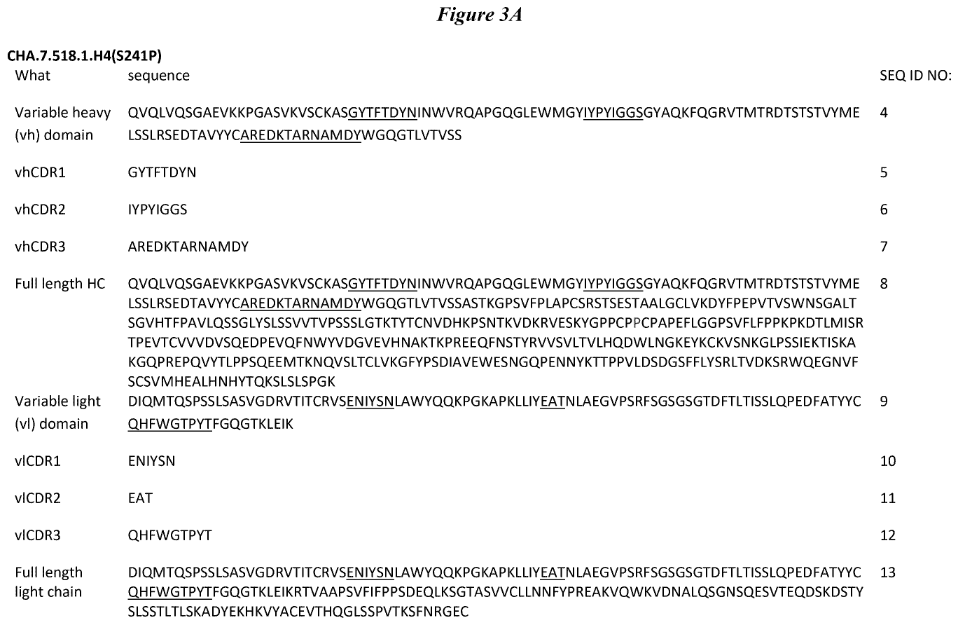

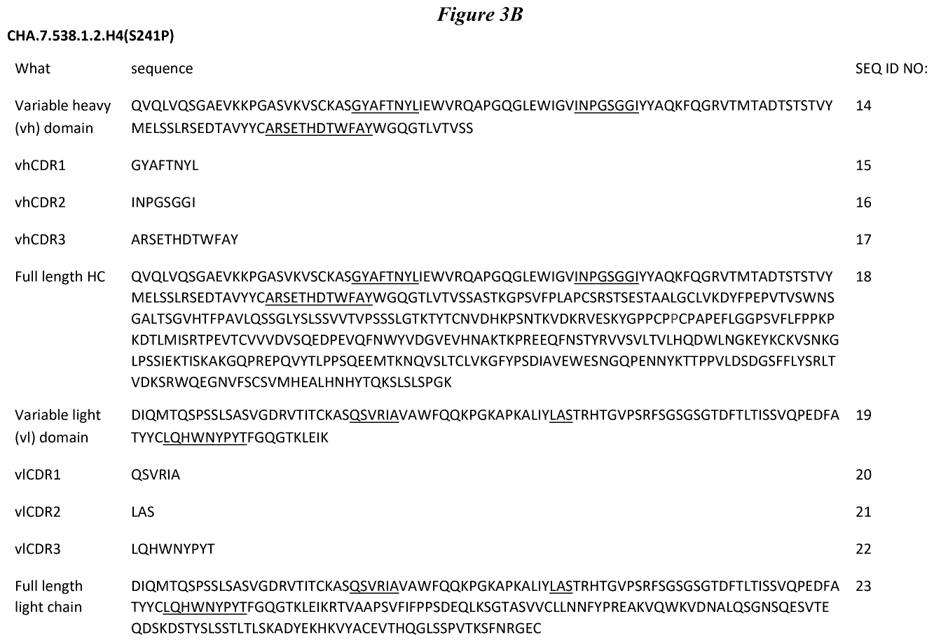

Accordingly, the invention provides anti-PVRIG antibodies that can be formulated according to the formulations described herein and which are provided in FIG. 3 (e.g., including anti-PVRIG antibodies including those with CDRs identical to those shown in FIG. 3). PVRIG, also called Poliovirus Receptor Related Immunoglobulin Domain Containing Protein, Q6DKI7 or C7orf15, relates to amino acid and nucleic acid sequences shown in RefSeq accession identifier NP_076975, shown in FIG. 1. The antibodies of the invention are specific for the PVRIG extracellular domain.

As is discussed below, the term “antibody” is used generally. Antibodies that find use in the present invention can take on a number of formats as described herein, including traditional antibodies as well as antibody derivatives, fragments and mimetics, described below. In general, the term “antibody” includes any polypeptide that includes at least one antigen binding domain, as more fully described below. Antibodies may be polyclonal, monoclonal, xenogeneic, allogeneic, syngeneic, or modified forms thereof, as described herein, with monoclonal antibodies finding particular use in many embodiments. In some embodiments, antibodies of the invention bind specifically or substantially specifically to PVRIG molecules. The terms “monoclonal antibodies” and “monoclonal antibody composition”, as used herein, refer to a population of antibody molecules that contain only one species of an antigen-binding site capable of immunoreacting with a particular epitope of an antigen, whereas the term “polyclonal antibodies” and “polyclonal antibody composition” refer to a population of antibody molecules that contain multiple species of antigen-binding sites capable of interacting with a particular antigen. A monoclonal antibody composition, typically displays a single binding affinity for a particular antigen with which it immunoreacts.

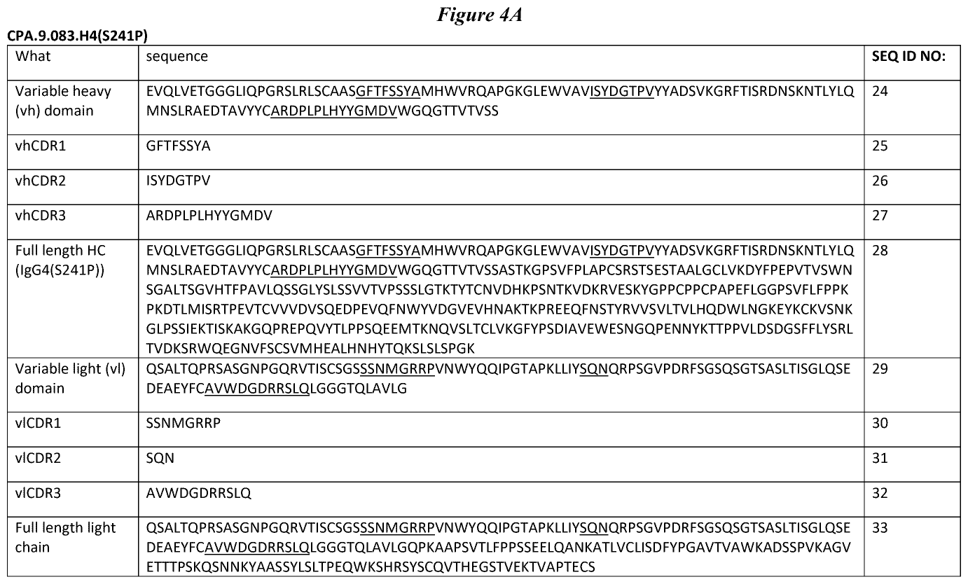

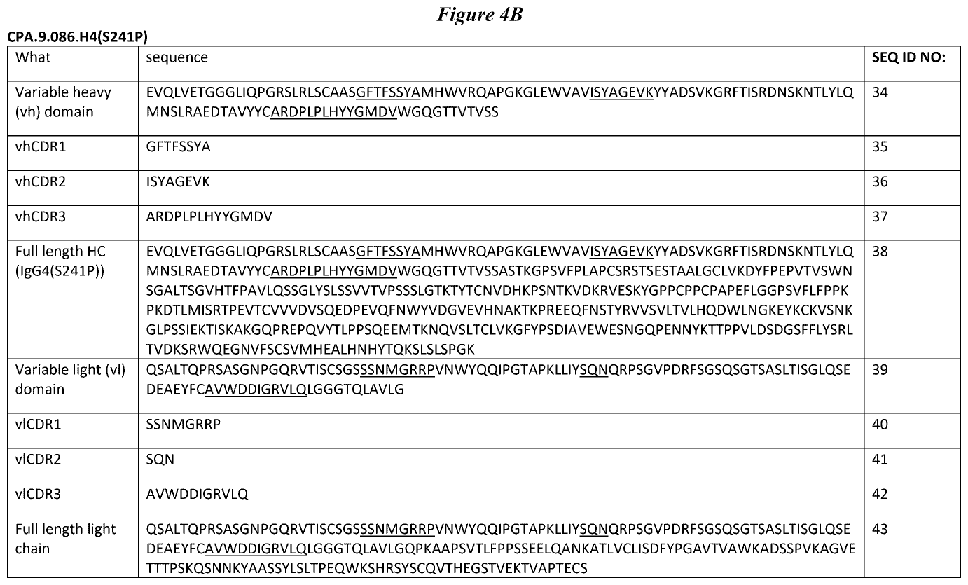

Traditional full length antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to the IgG class, which has several subclasses, including, but not limited to IgG1, IgG2, IgG3, and IgG4. Thus, “isotype” as used herein is meant any of the subclasses of immunoglobulins defined by the chemical and antigenic characteristics of their constant regions. While the exemplary antibodies herein designated “CPA” are based on IgG1 heavy constant regions, as shown in FIG. 4, the anti-PVRIG antibodies of the invention include those using IgG2, IgG3 and IgG4 sequences, or combinations thereof. For example, as is known in the art, different IgG isotypes have different effector functions which may or may not be desirable. Accordingly, the CPA antibodies of the invention can also swap out the IgG1 constant domains for IgG2, IgG3 or IgG4 constant domains (depicted in FIG. 4), with IgG2 and IgG4 finding particular use in a number of situations, for example for ease of manufacture or when reduced effector function is desired, the latter being desired in some situations.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions”.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

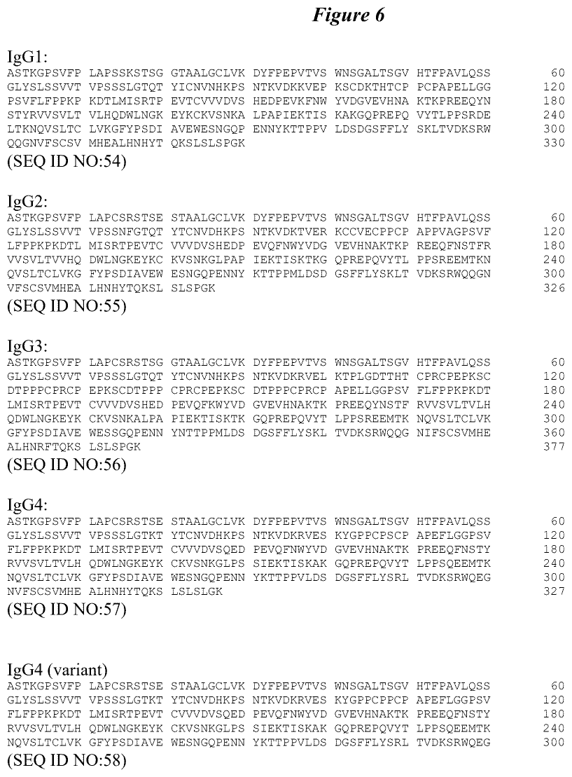

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region, although sometimes the numbering is shifted slightly as will be appreciated by those in the art; Kabat et al., SEQUENCES OF PROTEINS OF IMMUNOLOGICAL INTEREST, 5 th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below and shown in FIG. 6A-6D.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5 th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Accordingly, the invention provides variable heavy domains, variable light domains, heavy constant domains, light constant domains and Fc domains to be used as outlined herein. By “variable region” as used herein is meant the region of an immunoglobulin that comprises one or more Ig domains substantially encoded by any of the Vκ or Vλ, and/or VH genes that make up the kappa, lambda, and heavy chain immunoglobulin genetic loci respectively. Accordingly, the variable heavy domain comprises vhFR1-vhCDR1-vhFR2-vhCDR2-vhFR3-vhCDR3-vhFR4, and the variable light domain comprises vlFR1-vlCDR1-vlFR2- vlCDR2-vlFR3-vlCDR3-vlFR4. By “heavy constant region” herein is meant the CH1-hinge-CH2-CH3 portion of an antibody. By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

Thus, “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody. As will be appreciated by those in the art, these generally are made up of two chains.

Throughout the present specification, either the IMTG numbering system or the Kabat numbering system is generally used when referring to a residue in the variable domain (approximately, residues 1-107 of the light chain variable region and residues 1-113 of the heavy chain variable region) (e.g, Kabat et al., supra (1991)). EU numbering as in Kabat is generally used for constant domains and/or the Fc domains.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning”. Specific bins are described below.

Included within the definition of “antibody” is an “antigen-binding portion” of an antibody (also used interchangeably with “antigen-binding fragment”, “antibody fragment” and “antibody derivative”). That is, for the purposes of the invention, an antibody of the invention has a minimum functional requirement that it bind to a PVRIG antigen. As will be appreciated by those in the art, there are a large number of antigen fragments and derivatives that retain the ability to bind an antigen and yet have alternative structures, including, but not limited to, (i) the Fab fragment consisting of VL, VH, CL and CH1 domains, (ii) the Fd fragment consisting of the VH and CH1 domains, (iii) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (iv) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference), (v) “domain antibodies” or “dAb” (sometimes referred to as an “immunoglobulin single variable domain”, including single antibody variable domains from other species such as rodent (for example, as disclosed in WO 00/29004), nurse shark and Camelid V-HH dAbs, (vi) SMIPs (small molecule immunopharmaceuticals), camelbodies, nanobodies and IgNAR.

Still further, an antibody or antigen-binding portion thereof (antigen-binding fragment, antibody fragment, antibody portion) may be part of a larger immunoadhesion molecules (sometimes also referred to as “fusion proteins”), formed by covalent or noncovalent association of the antibody or antibody portion with one or more other proteins or peptides. Examples of immunoadhesion molecules include use of the streptavidin core region to make a tetrameric scFv molecule and use of a cysteine residue, a marker peptide and a C-terminal polyhistidine tag to make bivalent and biotinylated scFv molecules. Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

In general, the anti-PVRIG antibodies of the invention are recombinant. “Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “recombinant antibody”, as used herein, includes all antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

### A. Optional Antibody Engineering

The anti-PVRIG antibodies (e.g., anti-PVRIG antibodies including those with CDRs identical to those shown in FIG. 3) of the invention can be modified, or engineered, to alter the amino acid sequences by amino acid substitutions.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

As discussed herein, amino acid substitutions can be made to alter the affinity of the CDRs for the PVRIG protein (including both increasing and decreasing binding, as is more fully outlined below), as well as to alter additional functional properties of the antibodies. For example, the antibodies may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In some embodiments, amino acid substitutions can be made in the Fc region, in general for altering binding to FcγR receptors. By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII-1 (CD16), and FcγRIII-2 (CD16-2), as well as any undiscovered mouse FcγRs or FcγR isoforms or allotypes.

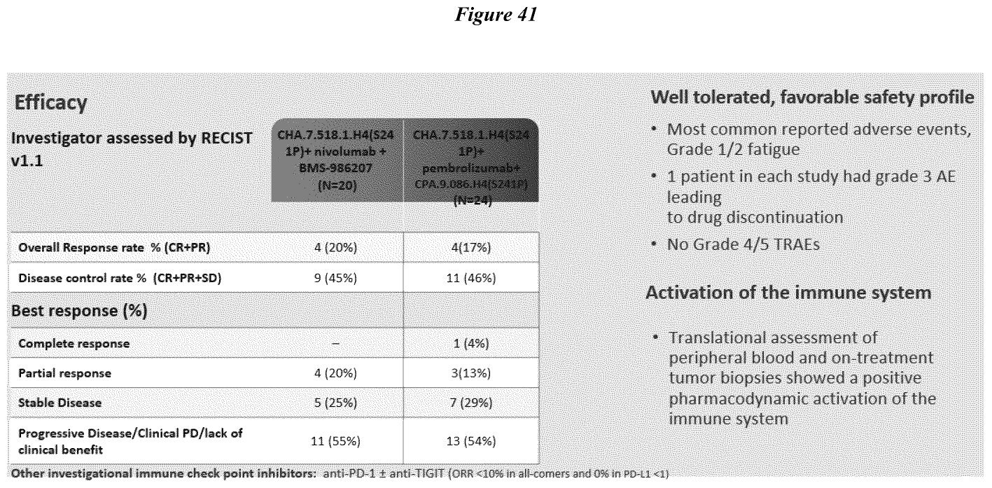

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell. Similarly, decreased binding to FcγRIIb (an inhibitory receptor) can be beneficial as well in some circumstances. Amino acid substitutions that find use in the present invention include those listed in U.S. Ser. Nos. 11/124,620 (particularly FIG. 41) and U.S. Pat. No. 6,737,056, both of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 299T and 297N.

In addition, the antibodies of the invention are modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or C_{L} region to contain a salvage receptor binding epitope taken from two loops of a CH2 domain of an Fc region of an IgG, as described in U.S. Pat. Nos. 5,869,046 and 6,121,022 by Presta et al. Additional mutations to increase serum half-life are disclosed in U.S. Pat. Nos. 8,883,973, 6,737,056 and 7,371,826, and include 428L, 434A, 434S, and 428L/434S.

In yet other embodiments, the Fc region is altered by replacing at least one amino acid residue with a different amino acid residue to alter the effector functions of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. Nos. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 are shown to improve binding to FcγRIII. Additionally, the following combination mutants are shown to improve FcγRIII binding: T256A/S298A, S298A/E333A, S298A/K224A and S298A/E333A/K334A. Furthermore, mutations such as M252Y/S254T/T256E or M428L/N434S improve binding to FcRn and increase antibody circulation half-life (see Chan C A and Carter P J (2010) Nature Rev Immunol 10:301-316).

In still another embodiment, the antibody can be modified to abrogate in vivo Fab arm exchange. Specifically, this process involves the exchange of IgG4 half-molecules (one heavy chain plus one light chain) between other IgG4 antibodies that effectively results in bispecific antibodies which are functionally monovalent. Mutations to the hinge region and constant domains of the heavy chain can abrogate this exchange (see Aalberse, RC, Schuurman J., 2002, Immunology 105:9-19).

In still another embodiment, the glycosylation of an antibody is modified. For example, an aglycosylated antibody can be made (i.e., the antibody lacks glycosylation). Glycosylation can be altered to, for example, increase the affinity of the antibody for antigen or reduce effector function such as ADCC. Such carbohydrate modifications can be accomplished by, for example, altering one or more sites of glycosylation within the antibody sequence, for example N297. For example, one or more amino acid substitutions can be made that result in elimination of one or more variable region framework glycosylation sites to thereby eliminate glycosylation at that site.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. For example, the cell lines Ms704, Ms705, and Ms709 lack the fucosyltransferase gene, FUT8 (α(1,6) fucosyltransferase), such that antibodies expressed in the Ms704, Ms705, and Ms709 cell lines lack fucose on their carbohydrates. The Ms704, Ms705, and Ms709 FUT8 cell lines are created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the a 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase α-L-fucosidase removes fucosyl residues from antibodies (Tarentino, A. L. et al. (1975) Biochem. 14:5516-23).

Another modification of the antibodies herein that is contemplated by the invention is pegylation or the addition of other water soluble moieties, typically polymers, e.g., in order to enhance half-life. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies according to at least some embodiments of the invention. See for example, EP 0 154 316 by Nishimura et al. and EP 0 401 384 by Ishikawa et al.

In addition to substitutions made to alter binding affinity to FcγRs and/or FcRn and/or increase in vivo serum half-life, additional antibody modifications can be made, as described in further detail below.

In some cases, affinity maturation is done. Amino acid modifications in the CDRs are sometimes referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

In some embodiments, one or more amino acid modifications are made in one or more of the CDRs of the PVRIG antibodies of the invention. In general, only 1 or 2 or 3-amino acids are substituted in any single CDR, and generally no more than from 1, 2, 3, 4, 5, 6, 7, 8 9 or 10 changes are made within a set of CDRs. However, it should be appreciated that any combination of no substitutions, 1, 2 or 3 substitutions in any CDR can be independently and optionally combined with any other substitution.

Affinity maturation can be done to increase the binding affinity of the antibody for the PVRIG antigen by at least about 10% to 50-100-150% or more, or from 1 to 5 fold as compared to the “parent” antibody. Preferred affinity matured antibodies will have nanomolar or even picomolar affinities for the PVRIG antigen. Affinity matured antibodies are produced by known procedures. See, for example, Marks et al., 1992, Biotechnology 10:779-783 that describes affinity maturation by variable heavy chain (VH) and variable light chain (VL) domain shuffling. Random mutagenesis of CDR and/or framework residues is described in: Barbas, et al. 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154(7):3310-9; and Hawkins et al, 1992, J. Mol. Biol. 226:889-896, for example.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of the enumerated antibodies of the invention. In addition, as outlined below, amino acid modifications can also independently and optionally be made in any region outside the CDRs, including framework and constant regions.

### V. PVRIG Antibodies

The present invention provides anti-PVRIG antibodies. (For convenience, “anti-PVRIG antibodies” and “PVRIG antibodies” are used interchangeably). The anti-PVRIG antibodies of the invention specifically bind to human PVRIG, and preferably the ECD of human PVRIG1, as depicted in FIG. 3, including, e.g., anti-PVRIG antibodies including those with CDRs identical to those shown in FIG. 3.

Specific binding for PVRIG or a PVRIG epitope can be exhibited, for example, by an antibody having a KD of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the PVRIG antigen or epitope.

However, as shown in the Examples, for optimal binding to PVRIG expressed on the surface of NK and T-cells, the antibodies preferably have a KD less 50 nM and most preferably less than 1 nM, with less than 0.1 nM and less than 1 pM and 0.1 pM finding use in the methods of the invention.

Also, specific binding for a particular antigen or an epitope can be exhibited, for example, by an antibody having a KA or Ka for a PVRIG antigen or epitope of at least 20-, 50-, 100-, 500-, 1000-, 5,000-, 10,000- or more times greater for the epitope relative to a control, where KA or Ka refers to an association rate of a particular antibody-antigen interaction. s

In some embodiments, the anti-PVRIG antibodies of the invention bind to human PVRIG with a K_{D} of 100 nM or less, 50 nM or less, 10 nM or less, or 1 nM or less (that is, higher binding affinity), or 1 pM or less, wherein K_{D} is determined by known methods, e.g. surface plasmon resonance (SPR, e.g. Biacore assays), ELISA, KINEXA, and most typically SPR at 250 or 37° C.

The invention provides antigen binding domains, including full length antibodies, which contain a number of specific, enumerated sets of 6 CDRs, as provided in FIG. 3. The invention provides antigen binding domains, including full length antibodies, which contain a number of specific, enumerated sets of 6 CDRs, as provided in FIG. 3.

The invention further provides variable heavy and light domains as well as full length heavy and light chains.

As discussed herein, the invention further provides variants of the above components, including variants in the CDRs, as outlined above. In addition, variable heavy chains can be at least 80%, at least 90%, at least 95%, at least 98% or at least 99% identical to the “VH” sequences herein, and/or contain from 1, 2, 3, 4, 5, 6, 7, 8, 9, 10 amino acid changes, or more, when Fc variants are used. Variable light chains are provided that can be at least 80%, at least 90%, at least 95%, at least 98% or at least 99% identical to the “VL” sequences herein, and/or contain from 1, 2, 3, 4, 5, 6, 7, 8, 9, 10 amino acid changes, or more, when Fc variants are used. Similarly, heavy and light chains are provided that are at least 80%, at least 90%, at least 95%, at least 98% or at least 99% identical to the “HC” and “LC” sequences herein, and/or contain from 1, 2, 3, 4, 5, 6, 7, 8, 9, 10 amino acid changes, or more, when Fc variants are used.

Accordingly, the present invention provides antibodies, usually full length or scFv domains, that comprise the following CHA sets of CDRs, the sequences of which are shown in FIG. 3:
CHA.7.518.1.H4(S241P)vhCDR1, CHA.7.518.1.H4(S241P)vhCDR2, CHA.7.518.1.H4(S241P)vhCDR3, CHA.7.518.1.H4(S241P)vlCDR1, CHA.7.518.1.H4(S241P)vlCDR2, and CHA.7.518.1.H4(S241P)vlCDR3.

In addition, the framework regions of the variable heavy and variable light chains can be humanized as is known in the art (with occasional variants generated in the CDRs as needed), and thus humanized variants of the VH and VL chains of FIG. 3 can be generated. Furthermore, the humanized variable heavy and light domains can then be fused with human constant regions, such as the constant regions from IgG1, IgG2, IgG3 and IgG4.

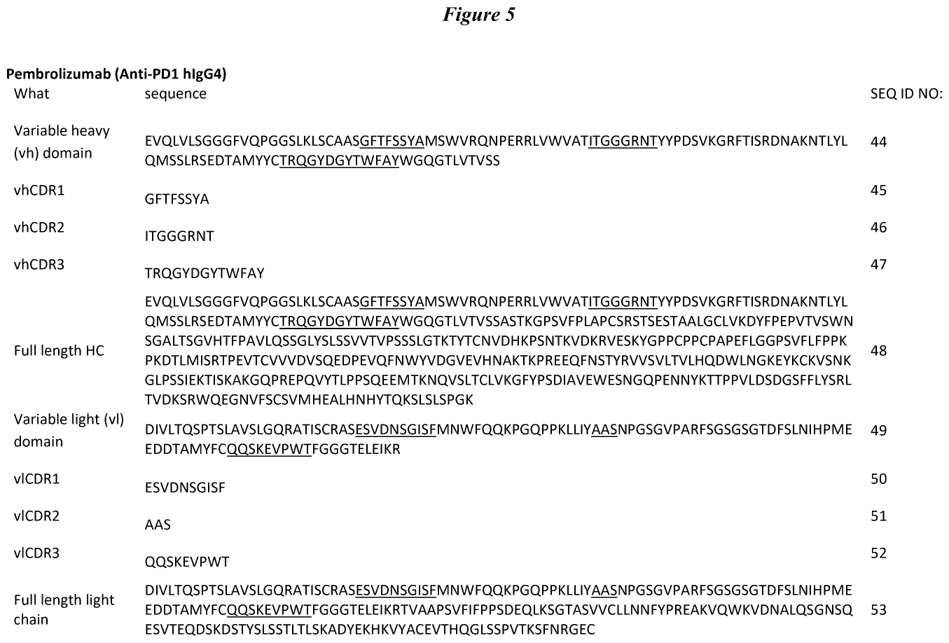

In addition, also included are sequences that may have the identical CDRs but changes in the variable domain (or entire heavy or light chain). For example, PVRIG antibodies include those with CDRs identical to those shown in FIG. 3 or FIGS. 5A-5D but whose identity along the variable region can be lower, for example 90%, 95% or 98% percent identical. For example, PVRIG antibodies include those with CDRs identical to those shown in FIG. 3 but whose identity along the variable region can be lower, for example 90%, 95% or 98% percent identical, and in some embodiments at least 90%, at least 95% or at least 98%.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In general, the percentage identity for comparison between PVRIG antibodies is at least 75%, at least 80%, at least 90%, with at least about 95, 96, 97, 98 or 99% percent identity being preferred. The percentage identity may be along the whole amino acid sequence, for example the entire heavy or light chain or along a portion of the chains. For example, included within the definition of the anti-PVRIG antibodies of the invention are those that share identity along the entire variable region (for example, where the identity is 95 or 98% identical along the variable regions, and in some embodiments at least 95% or at least 98%), or along the entire constant region, or along just the Fc domain.

### VI. TIGIT Antibodies

The present invention provides anti-TIGIT antibodies. (For convenience, “anti-TIGIT antibodies” and “TIGIT antibodies” are used interchangeably). The anti-TIGIT antibodies of the invention specifically bind to human TIGIT, and preferably the ECD of human TIGIT. The invention further provides antigen binding domains, including full length antibodies, which contain a number of specific, enumerated sets of 6 CDRs that bind to TIGIT, as shown for example in FIG. 4.

Specific binding for TIGIT or a TIGIT epitope can be exhibited, for example, by an antibody having a K_{D} of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, at least about 10⁻¹³ M, at least about 10⁻¹⁴ M, at least about 10⁻¹⁵ M, or greater, where K_{D} refers to the equilibrium dissociation constant of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the TIGIT antigen or epitope.

However, for optimal binding to TIGIT expressed on the surface of NK and T-cells, the antibodies preferably have a K_{D} less 50 nM and most preferably less than 1 nM, with less than 0.1 nM and less than 1 pM finding use in the methods of the invention

Also, specific binding for a particular antigen or an epitope can be exhibited, for example, by an antibody having a ka (referring to the association rate constant) for a TIGIT antigen or epitope of at least 20-, 50-, 100-, 500-, 1000-, 5,000-, 10,000- or more times greater for the epitope relative to a control, where ka refers to the association rate constant of a particular antibody-antigen interaction.

In some embodiments, the anti-TIGIT antibodies of the invention bind to human TIGIT with a K_{D} of 100 nM or less, 50 nM or less, 10 nM or less, or 1 nM or less (that is, higher binding affinity), or 1 pM or less, wherein K_{D} is determined by known methods, e.g. surface plasmon resonance (SPR, e.g. Biacore assays), ELISA, KINEXA, and most typically SPR at 250 or 37° C.

The invention provides antigen binding domains, including full length antibodies, which contain a number of specific, enumerated sets of 6 CDRs, as provided in FIG. 3. The invention provides antigen binding domains, including full length antibodies, which contain a number of specific, enumerated sets of 6 CDRs, as provided in FIG. 3.

The invention further provides variable heavy and light domains as well as full length heavy and light chains.

As discussed herein, the invention further provides variants of the above components, including variants in the CDRs, as outlined above. In addition, variable heavy chains can be at least 80%, at least 90%, at least 95%, at least 98% or at least 99% identical to the “VH” sequences herein, and/or contain from 1, 2, 3, 4, 5, 6, 7, 8, 9, 10 amino acid changes, or more, when Fc variants are used. Variable light chains are provided that can be at least 80%, at least 90%, at least 95%, at least 98% or at least 99% identical to the “VL” sequences herein, and/or contain from 1, 2, 3, 4, 5, 6, 7, 8, 9, 10 amino acid changes, or more, when Fc variants are used. Similarly, heavy and light chains are provided that are at least 80%, at least 90%, at least 95%, at least 98% or at least 99% identical to the “HC” and “LC” sequences herein, and/or contain from 1, 2, 3, 4, 5, 6, 7, 8, 9, 10 amino acid changes, or more, when Fc variants are used.

Accordingly, the present invention provides antibodies, usually full length or scFv domains, that comprise the following CHA sets of CDRs, the sequences of which are shown in FIG. 4:
CPA.9.083.H4(S241P)vhCDR1, CPA.9.083.H4(S241P)vhCDR2, CPA.9.083.H4(S241P)vhCDR3, CPA.9.083.H4(S241P) vlCDR1, CPA.9.083.H4(S241P) vlCDR2, and CPA.9.083.H4(S241P)vlCDR3; or
CPA. 9.086.H4(S241P)vhCDR1, CPA.9.086.H4(S241P)vhCDR2, CPA.9.086.H4(S241P)vhCDR3, CPA.9.086.H4(S241P) vlCDR1, CPA.9.086.H4(S241P) vlCDR2, and CPA.9.086.H4(S241P)vlCDR3; or

In addition, the framework regions of the variable heavy and variable light chains can be humanized as is known in the art (with occasional variants generated in the CDRs as needed), and thus humanized variants of the VH and VL chains of FIG. 4 can be generated. Furthermore, the humanized variable heavy and light domains can then be fused with human constant regions, such as the constant regions from IgG1, IgG2, IgG3 and IgG4.

In addition, also included are sequences that may have the identical CDRs but changes in the variable domain (or entire heavy or light chain). For example, TIGIT antibodies include those with CDRs identical to those shown in FIG. 4 but whose identity along the variable region can be lower, for example 95 or 98% percent identical. For example, TIGIT antibodies include those with CDRs identical to those shown in FIG. 5 but whose identity along the variable region can be lower, for example 95 or 98% percent identical, and in some embodiments at least 95% or at least 98%.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In general, the percentage identity for comparison between TIGIT antibodies is at least 75%, at least 80%, at least 90%, with at least about 95, 96, 97, 98 or 99% percent identity being preferred. The percentage identity may be along the whole amino acid sequence, for example the entire heavy or light chain or along a portion of the chains. For example, included within the definition of the anti-TIGIT antibodies of the invention are those that share identity along the entire variable region (for example, where the identity is 95 or 98% identical along the variable regions, and in some embodiments at least 95% or at least 98%), or along the entire constant region, or along just the Fc domain.

### A. Antibodies in Combination Therapy

The TIGIT and PVRIG antibodies of the invention find particular use in the treatment of cancer when used in combination with pembrolizumab, as described herein. The TIGIT and PVRIG antibodies of the invention find particular use in the treatment of cancer when used in combination with an anti-PD-1 antibody, as described herein. In general, the antibodies of the invention are immunomodulatory, in that rather than directly attack cancerous cells, the anti-TIGIT and anti-PVRIG antibodies of the invention stimulate the immune system, generally by inhibiting the action of TIGIT and PVRIG, respectively. Thus, unlike tumor-targeted therapies, which are aimed at inhibiting molecular pathways that are crucial for tumor growth and development, and/or depleting tumor cells, cancer immunotherapy is aimed to stimulate the patient's or subject's own immune system to eliminate cancer cells, providing long-lived tumor destruction. Various approaches can be used in cancer immunotherapy, among them are therapeutic cancer vaccines to induce tumor-specific T cell responses, and immunostimulatory antibodies (i.e. antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients or subjects respond (as opposed to conventional or targeted therapy, where a large number of patients or subjects respond, but responses are transient).

By the time a tumor is detected clinically, it has already evaded the immune-defense system by acquiring immunoresistant and immunosuppressive properties and creating an immunosuppressive tumor microenvironment through various mechanisms and a variety of immune cells.

Accordingly, the anti-TIGIT, anti-PVRIG, and pembrolizumab combination of the invention are useful in treating cancer. Due to the nature of an immuno-oncology mechanism of action, TIGIT and or PVRIG do not necessarily need to be overexpressed on or correlated with a particular cancer type; that is, the goal is to have the anti-TIGIT antibodies de-suppress T cell and NK cell activation, such that the immune system will go after the cancers.

In some embodiments, non-liming examples of the anti-PD-1 antibodies of the present invention include pembrolizumab, nivolumab (BMS-936558/MDX-1106/ONO-4538), cemiplimab, SHR-1210 (CTR20160175 and CTR20170090), SHR-1210 (CTR20170299 and CTR20170322), JS-001 (CTR20160274), IBI308 (CTR20160735), BGB-A317 (CTR20160872), AGEN2033w, AGEN2034w, AGEN2046w, AGEN2047w, AGEN2047w-N297A, AGEN2047w-S267E/L328F, AGEN2047w-S239D/A330L/I332E, AMP224, CT-011, and/or MK-3475. In some embodiments, the anti-PD-1 is pembrolizumab. In some embodiments, the anti-PD-1 is cemiplimab. In some embodiments, the anti-PD-1 is nivolumab (BMS-936558/MDX-1106/ONO-4538).

In some embodiments, non-liming examples of the anti-PD-1 antibodies of the present invention include pembrolizumab, cemiplimab, SHR-1210 (CTR20160175 and CTR20170090), SHR-1210 (CTR20170299 and CTR20170322), JS-001 (CTR20160274), IBI308 (CTR20160735), BGB-A317 (CTR20160872), AGEN2033w, AGEN2034w, AGEN2046w, AGEN2047w, AGEN2047w-N297A, AGEN2047w-S267E/L328F, AGEN2047w-S239D/A330L/I332E, AMP224, CT-011, and/or MK-3475. In some embodiments, the anti-PD-1 is pembrolizumab. In some embodiments, the anti-PD-1 is cemiplimab.

### VII. Formulations of Anti-PVRIG Antibodies

The anti-PVRIG antibodies and/or antigen binding portions thereof compositions (e.g., anti-PVRIG antibodies including those with CDRs identical to those shown in FIG. 3) can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's or subject's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980). Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™, or polyethylene glycol (PEG).

In a preferred embodiment, the pharmaceutical composition that comprises anti-PVRIG antibodies including those with CDRs identical to those shown in FIG. 3) of the invention may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferrably sterile. This is readily accomplished by filtration through sterile filtration membranes or other methods.

As used herein, the term “activity” refers to a functional activity or activities of anti-PVRIG antibodies and/or antigen binding portions thereof. Functional activities include, but are not limited to, biological activity and binding affinity.

As used herein, the term “stability” is used in a structural context, e.g., relating to the structural integrity of an anti-PVRIG antibody and/or antigen binding portion thereof, or in a functional context, e.g., relating to a an anti-PVRIG antibody and/or antigen binding portion thereof's ability to retain its function and/or activity over time (e.g., including anti-PVRIG antibody and/or antigen binding portion thereof stability or anti-PVRIG antibody and/or antigen binding portion thereof formulation stability, wherein the anti-PVRIG antibody includes those with CDRs identical to those shown in FIG. 3). As will be appreciated, the anti-PVRIG antibody and/or antigen binding portion thereof under discussion may be contained within a formulation in accordance with the methods and compositions described herein, and the stability of that protein refers to its stability in that formulation. In some embodiments, the stability of an anti-PVRIG antibody and/or antigen binding portion thereof composition is determined by measuring the binding activity of the composition, including for example, using the assays described in the application and figures provided herewith, as well as any other applicable assays known in the art. In some embodiments, the stability of an anti-PVRIG antibody and/or antigen binding portion thereof composition is formulated with sugar, sugar alcohol, and/or non-ionic surfactant, as described herein, is compared to an anti-PVRIG antibody and/or antigen binding portion thereof composition formulated without the at least one amino acid, salt, and/or non-ionic surfactant and/or with a different combination of components. In some embodiments, the formulation does not comprise a sugar and/or sugar alcohol.

As used herein, a “storage stable” aqueous an anti-PVRIG antibody and/or antigen binding portion thereof composition refers to a an anti-PVRIG antibody and/or antigen binding portion thereof comprising solution that has been formulated to increase the stability of the protein in solution, for example by at least 10%, over a given storage time. In the context of the present disclosure, an anti-PVRIG antibody and/or antigen binding portion thereof can be made “storage stable” by the addition of at least one amino acid, salt, or non-ionic surfactant as a stabilizing agent. In some embodiments, the stability of the anti-PVRIG antibody and/or antigen binding portion thereof in any given formulation can be measured, for example, by monitoring the formation of aggregates, loss of bulk binding activity, or formation of degradation products, over a period of time. The absolute stability of a formulation, and the stabilizing effects of the sugar, sugar alcohol, or non-ionic surfactant, will vary dependent upon the particular composition being stabilized. In one embodiment, the stability of an anti-PVRIG antibody and/or antigen binding portion thereof composition is determined by measuring the anti-PVRIG antibody and/or antigen binding portion thereof binding activity of the composition. For example, by using an ELISA or other binding activity assay. In one embodiment, the stability of an anti-PVRIG antibody and/or antigen binding portion thereof composition formulated with sugar, sugar alcohol, and/or non-ionic surfactant, as described herein, is compared to an anti-PVRIG antibody and/or antigen binding portion thereof composition formulated without the a least one amino acid, salt, and/or non-ionic surfactant and/or with a different combination of components. In some embodiments, the formulation does not comprise a sugar and/or sugar alcohol.

As used herein, “shelf-life” refers to the period of time a formulation maintains a predetermined level of stability at a predetermined temperature. In particular embodiments, the predetermined temperature refers to frozen (e.g., −80° C., −25° C., 0° C.), refrigerated (e.g., 0° to 10° C.), or room temperature (e.g., 18° C. to 32° C.) storage.

As used herein, the term “time of stability” refers to the length of time a formulation is considered stable. For example, the time of stability for a formulation may refer to the length of time for which the level of protein aggregation and/or degradation in the formulation remains below a certain threshold (e.g., 1%, 2%, 3%, 4%, 5%, 6%, 7%, 8%, 9%, 10%, 11%, 12%, 13%, 14%, 15%, 16%, 17%, 18%, 19%, 20%, etc.), and/or the length of time a formulation maintains biological activity above a certain threshold (e.g., 100%, 95%, 90%, 85%, 80%, 75%, 70%, 65%, 60%, 55%, 50%, etc.) of the amount of activity (including, for example, binding activity) present in the formulation at the start of the storage period.

In the context of the present disclosure, a storage stable aqueous composition of a an anti-PVRIG antibody and/or antigen binding portion thereof formulated with a sugar, sugar alcohol, and/or non-ionic surfactant will have a longer time of stability than a composition of the same an anti-PVRIG antibody and/or antigen binding portion thereof formulated without the at least one amino acid, salt, and/or non-ionic surfactant. In some embodiments, a storage stable aqueous composition of an anti-PVRIG antibody and/or antigen binding portion thereof, will have a time of stability that is, for example, at least 10% greater than the time of stability for an anti-PVRIG antibody and/or antigen binding portion thereof composition formulated in the absence of the at least one amino acid, salt, and/or non-ionic surfactant, or at least 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190% greater, or at least 2 times greater, or at least 2.5 times, 3.0 times, 3.5 times, 4.0 times, 4.5 times, 5.0 times, 5.5 times, 6.0 times, 6.5 times, 7.0 times, 7.5 times, 8.0 times, 8.5 times, 9.0 times, 9.5 times, 10 times, or more times greater than the time of stability for the anti-PVRIG antibody and/or antigen binding portion thereof composition formulated in the absence of the at least amino acid, salt, and/or non-ionic surfactant.

As used herein, “BDS” refers to “Bulk Drug Substance.”

### A. Stabilized Liquid Formulations

In some embodiments, the present disclosure provides stabilized aqueous formulations of an anti-PVRIG antibody and/or antigen binding portion thereof (e.g., anti-PVRIG antibodies including those with CDRs identical to those shown in FIG. 3). The following embodiments are based in part on the discovery that inclusion of at least one amino acid, salt, and/or non-ionic surfactant stabilizes the liquid anti-PVRIG antibody and/or antigen binding portion thereof compositions, as compared to compositions lacking the at least one amino acid, salt, and/or non-ionic surfactant. In some embodiments, the formulation does not comprise a sugar and/or sugar alcohol.

As will be recognized by one of skill in the art, an anti-PVRIG antibody and/or antigen binding portion thereof formulated according to the embodiments provided herein may contain, in addition to the components explicitly disclosed, counter ions contributed by the inclusion of solution components or pH modifying agents, for example, sodium or potassium contributed from an acetate salt, sodium hydroxide, or potassium hydroxide or chloride contributed by calcium chloride or hydrochloric acid. In the context of the present disclosure, a storage stable an anti-PVRIG antibody and/or antigen binding portion thereof composition consisting of or consisting essentially of a given formulation may further comprise one or more counter ion, as necessitated by the formulation process at a particular pH.

In one embodiment, a storage stable anti-PVRIG antibody and/or antigen binding portion provided herein will be stabilized at cryopreservation temperature (i.e., −20° C. or below) for a period of time. For example, in one embodiment, a stable liquid pharmaceutical formulations comprising an anti-PVRIG antibody or antigen binding fragment thereof will be stable when stored at cryopreservation temperature for at least 4 days. In other embodiments, the anti-PVRIG antibody and/or antigen binding portion composition will be stable at cryopreservation temperature for at least 1, 2, 3, 4, 5, 6, 7, 8, 9, 10, 11, 12, 13, 14, 21, 28, or more days. In some embodiments, the anti-PVRIG antibody and/or antigen binding portion composition will be stable for at least 1 week, 2 weeks, 3 weeks, 4 weeks, 5 weeks, 6 weeks, 7 weeks, 8 weeks, 9 weeks, 10 weeks, or more. In some embodiments, the anti-PVRIG antibody and/or antigen binding portion composition will be stable for at least 1 month. In some embodiments, the composition will be stable for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In some embodiments, the anti-PVRIG antibody and/or antigen binding portion composition will be stable for an extended period of time when stored at a temperature of about −20° C. or below.

In one embodiment, a storage stable anti-PVRIG antibody and/or antigen binding portion provided herein will be stabilized at refrigerated temperature (i.e., between 2° C. and 10° C.) for a period of time. For example, in one embodiment, a stable liquid pharmaceutical formulations comprising an anti-PVRIG antibody or antigen binding fragment thereof will be stable when stored at refrigerated temperature for at least 4 days. In other embodiments, the anti-PVRIG antibody and/or antigen binding portion composition will be stable at refrigerated temperature for at least 1, 2, 3, 4, 5, 6, 7, 8, 9, 10, 11, 12, 13, 14, 21, 28, or more days. In some embodiments, the anti-PVRIG antibody and/or antigen binding portion composition will be stable for at least 1 week, 2 weeks, 3 weeks, 4 weeks, 5 weeks, 6 weeks, 7 weeks, 8 weeks, 9 weeks, 10 weeks, or more. In some embodiments, the anti-PVRIG antibody and/or antigen binding portion composition will be stable for at least 1 month. In some embodiments, the composition will be stable for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In some embodiments, the anti-PVRIG antibody and/or antigen binding portion composition will be stable for an extended period of time when stored at a temperature between 2° C. and 8° C.

In one embodiment, a stable liquid pharmaceutical formulations comprising an anti-PVRIG antibody or antigen binding fragment thereof provided herein will be stabilized at room temperature (i.e., between 18° C. and 32° C.) for a period of time. For example, in one embodiment, a stable liquid pharmaceutical formulations comprising an anti-PVRIG antibody or antigen binding fragment thereof will be stable when stored at room temperature for at least 4 days. In some embodiments, the anti-PVRIG antibody and/or antigen binding portion composition will be stable at room temperature for at least 1, 2, 3, 4, 5, 6, 7, 8, 9, 10, 11, 12, 13, 14, 21, 28, or more days. In some embodiments, the anti-PVRIG antibody and/or antigen binding portion composition will be stable for at least 1 week, 2 weeks, 3 weeks, 4 weeks, 5 weeks, 6 weeks, or more. In some embodiments, the anti-PVRIG antibody and/or antigen binding portion composition will be stable for at least 1 month. In yet other embodiments, the composition will be stable for at least 2, 3, 4, 5, 6, 7, 8, 9, 10, 11, 12, 13, 14, 15, 16, 17, 18, 19,20,21,22,23,24,25,26,27,28,29,30,31,32,33,34,35,36,37,38,39,40,41,42,43, 44, 45, 46, 47, 48, or more months. In some embodiments, room temperature refers to between 20° C. and 30° C., between 21° C. and 29° C., between 22° C. and 28° C., between 23° C. and 27° C., between 24° C. and 26° C., or about 25° C. In some embodiments, the anti-PVRIG antibody and/or antigen binding portion composition will be stable for an extended period of time when stored at a temperature between 20° C. and 25° C. In some embodiments, the anti-PVRIG antibody and/or antigen binding portion composition will be stable for an extended period of time when stored at a temperature of about 25° C.

In one embodiment, a storage stable anti-PVRIG antibody and/or antigen binding portion provided herein will be stabilized at elevated temperature (i.e., between 32° C. and 42° C.) for a period of time. For example, in one embodiment, a stable liquid pharmaceutical formulations comprising an anti-PVRIG antibody or antigen binding fragment thereof will be stable when stored at elevated temperature for at least 4 days. In some embodiments, the anti-PVRIG antibody and/or antigen binding portion composition will be stable at elevated temperature for at least 1, 2, 3, 4, 5, 6, 7, 8, 9, 10, 11, 12, 13, 14, 21, 28, or more days. In some embodiments, the anti-PVRIG antibody and/or antigen binding portion composition will be stable for at least 1 week, 2 weeks, 3 weeks, 4 weeks, 5 weeks, 6 weeks, 7 weeks, 8 weeks, or more. In some embodiments, the anti-PVRIG antibody and/or antigen binding portion composition will be stable for at least 1 month. In yet other embodiments, the anti-PVRIG antibody and/or antigen binding portion composition will be stable for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more months. In some embodiments, the anti-PVRIG antibody and/or antigen binding portion composition will be stable for an extended period of time when stored at a temperature between 35° C. and 40° C.

In one embodiment, a stored anti-PVRIG antibody and/or antigen binding portion composition is considered storage stable as long as the composition maintains at least 40% of the antibody binding activity present at the start of the storage period (e.g., at time=0). In another embodiment, a stored composition is considered stable as long as the composition maintains at least 45%, 50%, 55%, 60%, 65%, 70%, 75%, 80%, 85%, 90%, 95% or more of the antibody binding activity present at the start of the storage period (e.g., at time=0). In one embodiment, antibody binding activity is measure using any assay known in the art.

In some embodiments, an anti-PVRIG antibody and/or antigen binding portion composition is considered to have been stabilized by the addition of a stabilizing agent (e.g., at least one amino acid, salt, and/or non-ionic surfactant) when the anti-PVRIG antibody and/or antigen binding portion composition contains at least 10% more antibody binding activity after storage for a period of time, as compared to an anti-PVRIG antibody and/or antigen binding portion composition not containing the stabilizing agent or containing a lower amount of the stabilizing agent. In other embodiments, an anti-PVRIG antibody and/or antigen binding portion composition is considered to have been stabilized by the addition of a stabilizing agent (e.g., at least one amino acid, salt, and/or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage more anti-PVRIG antibody and/or antigen binding portion activity after storage for a period of time, as compared to an anti-PVRIG antibody and/or antigen binding portion composition not containing the stabilizing agent or containing a lower amount of the stabilizing agent.

In one embodiment, a stored anti-PVRIG antibody and/or antigen binding portion composition is considered stable as long as the percentage of anti-PVRIG antibody and/or antigen binding portion present in an aggregated state remains no more than 50%. In some embodiments, a stored anti-PVRIG antibody and/or antigen binding portion thereof composition is considered stable as long as the percentage of the anti-PVRIG antibody and/or antigen binding portion thereof present in an aggregated state remains no more than 45%, 40%, 35%, 30%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less.

In some embodiments, an anti-PVRIG antibody and/or antigen binding portion composition is considered to have been stabilized by the addition of a stabilizing agent (anti-PVRIG antibody and/or antigen binding portion composition, at least one amino acid, salt, and/or non-ionic surfactant) when the composition contains at least 10% less anti-PVRIG antibody and/or antigen binding portion present in an aggregated state after storage for a period of time, as compared to an anti-PVRIG antibody and/or antigen binding portion composition not containing the stabilizing agent or containing a lower amount of the stabilizing agent. In other embodiments, an anti-PVRIG antibody and/or antigen binding portion composition is considered to have been stabilized by the addition of a stabilizing agent (e.g., at least one amino acid, salt, and/or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage less anti-PVRIG antibody and/or antigen binding portion present in an aggregated state after storage for a period of time, as compared to an anti-PVRIG antibody and/or antigen binding portion composition not containing the stabilizing agent or containing a lower amount of the stabilizing agent

In some embodiments, a stored anti-PVRIG antibody and/or antigen binding portion composition is considered stable as long as the composition maintains at least 40% of the starting binding activity (e.g., at time=0) after being subjected to mechanical stress. In another embodiment, a stored composition is considered stable as long as the composition maintains 45%, 50%, 55%, 60%, 65%, 70%, 75%, 80%, 85%, 90%, 95% or more of the starting binding activity (e.g., at time=0) after being subjected to mechanical stress. In some embodiments, the mechanical stress is agitation (e.g., shaking).

In some embodiments, an anti-PVRIG antibody and/or antigen binding portion composition is considered to have been stabilized by the addition of a stabilizing agent (e.g., at least one amino acid, salt, or non-ionic surfactant) when the anti-PVRIG antibody and/or antigen binding portion composition contains at least 10% more binding activity after being subjected to mechanical stress, as compared to an anti-PVRIG antibody and/or antigen binding portion composition not containing the stabilizing agent or containing a lower amount of the stabilizing agent. In other embodiments, an anti-PVRIG antibody and/or antigen binding portion composition is considered to have been stabilized by the addition of a stabilizing agent (e.g., a sugar, sugar alcohol, or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage more furin activity after being subjected to mechanical stress, as compared to an anti-PVRIG antibody and/or antigen binding portion composition not containing the stabilizing agent or containing a lower amount of the stabilizing agent. In a specific embodiment, the mechanical stress is agitation (e.g., shaking).

In some embodiments, a stored anti-PVRIG antibody and/or antigen binding portion composition is considered stable as long as the percentage of anti-PVRIG antibody and/or antigen binding portion present in an aggregated state remains no more than 50% after being subjected to mechanical stress. In other embodiments, a stored anti-PVRIG antibody and/or antigen binding portion composition is considered stable as long as the percentage of anti-PVRIG antibody and/or antigen binding portion present in an aggregated state remains no more than 45%, 40%, 35%, 30%, 25%, 24%, 23%, 22%, 21%, 20%, 19%, 18%, 17%, 16%, 15%, 4%1, 3%1, 12%, 11%, 10%, 9%, 8%, 7%, 6%, 5%, 4%, 3%, 2%, 1%, or less after being subjected to mechanical stress. In some embodiments, the mechanical stress is agitation (e.g., shaking).

In some embodiments, an anti-PVRIG antibody and/or antigen binding portion composition is considered to have been stabilized by the addition of a stabilizing agent (e.g., at least one amino acid, salt, or non-ionic surfactant) when the composition contains at least 10% less anti-PVRIG antibody and/or antigen binding portion present in an aggregated state after being subjected to mechanical stress, as compared to an anti-PVRIG antibody and/or antigen binding portion composition not containing the stabilizing agent or containing a lower amount of the stabilizing agent. In some embodiments, an anti-PVRIG antibody and/or antigen binding portion composition is considered to have been stabilized by the addition of a stabilizing agent (e.g., at least one amino acid, salt, or non-ionic surfactant) when the composition contains at least 15%, 20%, 25%, 30%, 35%, 40%, 45%, 50%, 55%, 60%, 65%, 70%, 75%, 80%, 85%, 90%, 95%, 100%, or a greater percentage less anti-PVRIG antibody and/or antigen binding portion present in an aggregated state after being subjected to mechanical stress, as compared to an anti-PVRIG antibody and/or antigen binding portion composition not containing the stabilizing agent or containing a lower amount of the stabilizing agent. In a specific embodiment, the mechanical stress is agitation (e.g., shaking).

In some embodiments, the highly stabilized formulations of the invention have a shelf life of at least 6 months. As will be appreciated, this shelf life may be at frozen temperatures (i.e., −80° C., −25° C., 0° C.), refrigerated (0° C. to 10° C.), or room temperature (20° C. to 32° C.) in liquid or lyophilized form. In further aspects, the highly stabilized formulations of the invention have a shelf life of at least 12, 18, 24, 30, 36, 42, 48, 54, or 60 months.

In some embodiments, shelf life is determined by a percent activity remaining after storage at any of the above temperatures for any of the above periods of time. In some embodiments, shelf life means that the formulation retains at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100% of furin activity as measured by any of the assays described herein or known in the art as compared to activity prior to storage for any of the above amounts of time at any of the above temperatures.

In some embodiments, the present invention provides a stable liquid pharmaceutical formulation of an anti-PVRIG antibody comprising:
(a) an anti-PVRIG antibody, wherein the anti-PVRIG antibody comprises an antibody with CDRs identical to those shown in FIG. 3;
(b) 25 mM histidine;
(c) 60 mM NaCl;
(d) 100 mM L-Arginine, and
(e) 0.01% w/v polysorbate 80,
wherein the composition has a pH from 5.5 to 7.0.

In some embodiments, the anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL. In some embodiments, the anti-PVRIG antibody is at a concentration of from 10 mg/mL to 40 mg/mL. In some embodiments, the anti-PVRIG antibody is at a concentration of from 15 mg/mL to 40 mg/mL. In some embodiments, the anti-PVRIG antibody is at a concentration of from 15 mg/mL to 30 mg/mL. In some embodiments, the anti-PVRIG antibody is at a concentration of from 10 mg/mL to 25 mg/mL. In some embodiments, the anti-PVRIG antibody is at a concentration of from 15 mg/mL to 25 mg/mL. In some embodiments, the anti-PVRIG antibody is at a concentration of from 10 mg/mL to 25 mg/mL. In some embodiments, the anti-PVRIG antibody is at a concentration of from 15 mg/mL to 25 mg/mL. In some embodiments, the anti-PVRIG antibody is at a concentration of from 20 mg/mL to 25 mg/mL. In some embodiments, the anti-PVRIG antibody is at a concentration of about 20 mg/mL.

In some embodiments, the present invention provides a stable liquid pharmaceutical formulation of an anti-PVRIG antibody comprising:
(a) an anti-PVRIG antibody, wherein the anti-PVRIG antibody comprises an antibody with CDRs identical to those shown in FIG. 3;
(b) from 10 mM to 100 mM histidine;
(c) from 30 mM to 100 mM NaCl;
(d) from 20 mM to 150 mM L-Arginine, and
(e) from 0.005% to 0.1% w/v polysorbate 80
wherein the composition has a pH from 5.5 to 7.0.

### B. Amino Acids

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising at least one amino acid. In some embodiments, the at least one amino acid is histidine. In some embodiments, the at least one amino acid is arginine. In some embodiments, the present invention provides a stable liquid pharmaceutical formulation of an anti-PVRIG antibody or antigen binding fragment thereof comprising at least two amino acids. In some embodiments, the at least two amino acids are histidine and arginine.

In some embodiments, the pharmaceutical formulation comprises from 10 mM to 80 mM histidine, from 15 mM to 70 mM histidine, from 20 mM to 60 mM histidine, from 20 mM to 50 mM histidine, or from 20 mM to 30 mM histidine. In some embodiments, the pharmaceutical formulation comprises from 10 mM to 80 mM histidine. In some embodiments, the pharmaceutical formulation comprises from 15 mM to 70 mM histidine. In some embodiments, the pharmaceutical formulation comprises from 20 mM to 60 mM histidine. In some embodiments, the pharmaceutical formulation comprises from 20 mM to 50 mM histidine. In some embodiments, the pharmaceutical formulation comprises from 20 mM to 30 mM histidine. In some embodiments, the pharmaceutical formulation comprises about 25 mM histidine.

In some embodiments, the pharmaceutical formulation comprises from 10 mM to 80 mM histidine. In some embodiments, the pharmaceutical formulation comprises from 15 mM to 70 mM histidine. In some embodiments, the pharmaceutical formulation comprises from 20 mM to 60 mM histidine. In some embodiments, the pharmaceutical formulation comprises from 20 mM to 50 mM histidine. In some embodiments, the pharmaceutical formulation comprises from 20 mM to 30 mM histidine. In some embodiments, the pharmaceutical formulation comprises about 25 mM histidine.

In some embodiments, the pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine. In some embodiments, the pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, or from 90 mM to 110 mM L-arginine.

In some embodiments, the pharmaceutical formulation comprises from 20 mM to 140 mM L-arginine. In some embodiments, the pharmaceutical formulation comprises from 30 mM to 140 mM L-arginine. In some embodiments, the pharmaceutical formulation comprises from 40 mM to 130 mM L-arginine. In some embodiments, the pharmaceutical formulation comprises from 50 mM to 120 mM L-arginine. In some embodiments, the pharmaceutical formulation comprises from 60 mM to 110 mM L-arginine. In some embodiments, the pharmaceutical formulation comprises from 70 mM to 110 mM L-arginine. In some embodiments, the pharmaceutical formulation comprises from 80 mM to 110 mM L-arginine. In some embodiments, the pharmaceutical formulation comprises from 90 mM to 110 mM L-arginine. In some embodiments, the pharmaceutical formulation comprises about 100 mM L-arginine.

C. Sugar and/or Sugar Alcohol

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising no sugar and/or sugar alcohol. In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising no sugar. In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising no sugar alcohol.

In some embodiments, the present invention provides a stable liquid pharmaceutical formulation of an anti-PVRIG antibody or antigen binding fragment thereof comprising a sugar and/or sugar alcohol. In some embodiments, the sugar is trehalose or sucrose. In some embodiments, the sugar is trehalose. In some embodiments, the sugar is sucrose. In some embodiments, the sugar is only one of trehalose or sucrose but not both.

In some embodiments, the sugar is in an amount of from about 0.5% to 10%, 1% to 9.5%, 1.5% to 9%, 2.0% to 8.5%, 2.5% to 8%, 3.0% to 7.5%, 3.5% to 7%, 4.0% to 6.5%, 4.5% to 6%, and/or 4.5% to 5.5%. In some embodiments, the sugar is in an amount of from about 0.5% to 10%. In some embodiments, the sugar is in an amount of from about 1% to 9.5%. In some embodiments, the sugar is in an amount of from about 1.5% to 9%. In some embodiments, the sugar is in an amount of from about 2.0% to 8.5%. In some embodiments, the sugar is in an amount of from about 2.5% to 8%. In some embodiments, the sugar is in an amount of from about 3.0% to 7.5%. In some embodiments, the sugar is in an amount of from about 3.5% to 7%. In some embodiments, the sugar is in an amount of from about 4.0% to 6.5%. In some embodiments, the sugar is in an amount of from about 4.5% to 6%. In some embodiments, the sugar is in an amount of from about 4.5% to 5.5%. In some embodiments, the sugar is in an amount of about 5%

### D. Non-Ionic Surfactants

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising a non-ionic surfactant. In some embodiments, the storage stable compositions of an anti-PVRIG antibody or antigen binding fragment comprise a non-ionic surfactant selected from a non-ionic water soluble monoglyceride, a non-ionic water soluble diglyceride, a non-ionic water soluble triglyceride, a non-ionic water soluble monofatty acid esters of polyethyelene glycol, a non-ionic water soluble difatty acid esters of polyethyelene glycol, a non-ionic water soluble sorbitan fatty acid ester, a non-ionic polyglycolyzed glyceride, a non-ionic water soluble triblock copolymer, and a combination thereof. In some embodiments, the non-ionic surfactant is polysorbate 80 (polyoxyethylene (20) sorbitan monooleate).

In some embodiments, the stable liquid pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, or from 0.009% to 0.05% w/v polysorbate 80. In some embodiments, the stable liquid pharmaceutical formulation comprises from 0.006% to 0.1% w/v polysorbate 80. In some embodiments, the stable liquid pharmaceutical formulation comprises from 0.007% to 0.09% w/v polysorbate 80. In some embodiments, the stable liquid pharmaceutical formulation comprises from 0.008% to 0.08% w/v polysorbate 80. In some embodiments, the stable liquid pharmaceutical formulation comprises from 0.009% to 0.09% w/v polysorbate 80. In some embodiments, the stable liquid pharmaceutical formulation comprises from 0.01% to 0.08% w/v polysorbate 80. In some embodiments, the stable liquid pharmaceutical formulation comprises from 0.01% to 0.07% w/v polysorbate 80. In some embodiments, the stable liquid pharmaceutical formulation comprises from 0.01% to 0.07% w/v polysorbate 80. In some embodiments, the stable liquid pharmaceutical formulation comprises from 0.01% to 0.06% w/v polysorbate 80. In some embodiments, the stable liquid pharmaceutical formulation comprises from 0.009% to 0.05% w/v polysorbate 80. In some embodiments, the stable liquid pharmaceutical formulation comprises about 0.01% polysorbate 80.

### E. Pharmaceutically Acceptable Salts

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) comprising a salt, for example, a pharmaceutically acceptable salt.

In some embodiments, the stable liquid pharmaceutical formulation comprising an anti-PVRIG antibody or antigen binding fragment thereof provided herein include a pharmaceutically acceptable salt at a concentration tolerated by an anti-PVRIG antibody or antigen binding fragment thereof during storage. In some embodiments, the pharmaceutically acceptable salt is a chloride salt. In some embodiments, the pharmaceutically acceptable salt is a monovalent chloride salt. In a more specific embodiment, the pharmaceutically acceptable salt is sodium chloride, potassium chloride, or a combination thereof.

In some embodiments, the stable liquid pharmaceutical formulation comprises from 30 mM to 100 mM NaCl, from 30 mM to 90 mM NaCl, from 40 mM to 80 mM NaCl, from 30 mM to 70 mM histidine, or from 45 mM to 70 mM NaCl.

In some embodiments, the stable liquid pharmaceutical formulation comprises from 30 mM to 100 mM NaCl. In some embodiments, the stable liquid pharmaceutical formulation comprises from 30 mM to 90 mM NaCl. In some embodiments, the stable liquid pharmaceutical formulation comprises from 40 mM to 80 mM NaCl. In some embodiments, the stable liquid pharmaceutical formulation comprises from 30 mM to 70 mM histidine. In some embodiments, the stable liquid pharmaceutical formulation comprises or from 45 mM to 70 mM NaCl. In some embodiments, pharmaceutical formulation comprises about 60 mM NaCl.

### F. Buffering Agents

In some embodiments, the present invention provides a stable liquid pharmaceutical formulation of an anti-PVRIG antibody or antigen binding fragment thereof (e.g., anti-PVRIG antibodies including those with CDRs identical to those shown in FIG. 3) that is buffered at a physiologically acceptable pH. In some embodiments, the physiologically acceptable pH is from about 6.0 to about 7.0.

In some embodiments, stable liquid pharmaceutical formulation of an anti-PVRIG antibody or antigen binding fragment thereof has a pH of from 6 to 7.0. In some embodiments, stable liquid pharmaceutical formulation of an anti-PVRIG antibody or antigen binding fragment thereof has a pH of 6, 6.1, 6.2, 6.3, 6.4, 6.5, 6.6, 6.7, 6.8, 6.9, or 7.0. In some embodiments, the pH is from 6.1 to 6.9. In some embodiments, the pH is from 6.2 to 6.9. In some embodiments, the pH is from 6.3 to 6.8. In some embodiments, the pH is from 6.3 to 6.7. In some embodiments, the pH is from 6.4 to 6.8. In some embodiments, the pH is from 6.5 to 6.8. In some embodiments, the pH is from 6.6 to 6.8. In some embodiments, the pH is 6.3, 6.4, 6.5, 6.6, or 6.7. In some embodiments, the pH is 6.5+/−0.2.

### G. Methods for Diluting Aqueous Compositions

[In some embodiments, the method includes adding a dilution buffer, to form a diluted stable liquid pharmaceutical formulation comprising an anti-PVRIG antibody or antigen binding fragment thereof (e.g., anti-PVRIG antibodies including those with CDRs identical to those shown in FIG. 3). In some embodiments, the dilution buffer is added at a ratio of from 1:1 (dilution buffer:formulation) to 1000:1 (dilution buffer:formulation). In another embodiment, the dilution buffer is added at a ratio of from 1:1 dilution buffer:formulation) to 500:1 (dilution buffer:formulation). In another embodiment, the dilution buffer is added at a ratio of from 1:1 (dilution buffer:formulation) to 250:1 (dilution buffer:formulation). In another embodiment, the dilution buffer is added at a ratio of from 1:1 (dilution buffer:formulation) to 200:1 (dilution buffer:formulation). In another embodiment, the dilution buffer is added at a ratio of from 1:1 (dilution buffer:formulation) to 100:1 (dilution buffer:formulation). In another embodiment, the dilution buffer is added at a ratio of from 1:1 (dilution buffer:formulation) to 50:1 (dilution buffer:formulation).

In some embodiments, the stable liquid pharmaceutical formulation comprising an anti-PVRIG antibody or antigen binding fragment thereof is diluted from 1-fold to 1000-fold, from 1-fold to 500-fold, from 1-fold to 250-fold, from 1-fold to 200-fold, from 1-fold to 100-fold, from 1-fold to 50-fold, from 1-fold to 10-fold, from 10-fold to 1000-fold, from 10-fold to 500-fold, from 10-fold to 250-fold, from 10-fold to 200-fold, from 10-fold to 100-fold, from 10-fold to 50-fold, from 50-fold to 1000-fold, from 50-fold to 500-fold, from 50-fold to 250-fold, from 50-fold to 200-fold, from 50-fold to 100-fold, from 100-fold to 1000-fold, from 100-fold to 500-fold, from 100-fold to 250-fold, from 100-fold to 200-fold, from 200-fold to 1,000-fold, from 200-fold to 500-fold, or from 200-fold to 250-fold.

### H. Stability Assays

As discussed herein, the stable liquid pharmaceutical formulations comprising an anti-PVRIG antibody or antigen binding fragment thereof (e.g., anti-PVRIG antibodies including those with CDRs identical to those shown in FIG. 3) show improved stability as compared to control formulations. In one embodiment, improved stability includes retention of a higher percentage of binding activity and/or no reduction in binding activity as compared to control formulations in various stability assays. Such assays can be used to determine if a formulation is a highly stabilized formulation. In some embodiments, the highly stabilized formulation has at least 5%, 10%, 20%, 30%, 40%, 50%, 55%, 60%, 65%, 70%, 75%, 80%, 85%, 90%, 95%, 96%, 97%, 98%, 99% or greater activity than a control formulation when assessed by any of the stability assays discussed herein or known in the art.

In some embodiments, the liquid pharmaceutical formulations comprising an anti-PVRIG antibody or antigen binding fragment thereof are tested under stressor conditions, such as storage at high temperature, agitation, freeze/thaw cycles, or some combination thereof. After such stressors, the formulations are assayed using any of the methods described herein or known in the art to determine the stability under these conditions.

### A280 and Appearance Analysis

In some embodiments, an A280 by SoloVPE assay is used to examine the appearance of the stable liquid pharmaceutical formulations comprising an anti-PVRIG antibody or antigen binding fragment thereof.

In some embodiments, the SoloVPE assay can be employed to examine concentrations for the stable liquid pharmaceutical formulations comprising an anti-PVRIG antibody or antigen binding fragment thereof.

A280: Amino acids containing aromatic side chains exhibit strong UV-light absorption at the wavelength of 280 nm. Once an absorptivity coefficient has been established for a given protein, the protein's concentration in solution can be calculated from its absorbance. The method is designed to determine the protein concentration by measuring its absorbance at 280 nm using the SoloVPE instrument without dilution (https://www.ctechnologiesinc.com/products/solovpe)

Appearance: Sample appearance determination is assessed by holding the sample within a controlled light source and observe the appearance of the material. Gently agitate the solution and determine if the appearance changes when viewed against a black and white background. Use adjectives such as “clear”, “turbid”, or “slightly turbid” to assess clarity. Be specific with regards to the color of the material. If the material is colorless then state that as a result (i.e. clear, colorless solution). specify the physical state of the sample (i.e. liquid or frozen liquid)

### Binding Assay Analysis

In some embodiments, a binding assay can be performed to examine the activity of the stable liquid pharmaceutical formulations comprising an anti-PVRIG antibody or antigen binding fragment thereof.

### LabChip Analysis

In some embodiments, a LabChip analysis is employed to examine purity, including for example, IgG purity as well as HC+LC percentages for the stable liquid pharmaceutical formulations comprising an anti-PVRIG antibody or antigen binding fragment thereof. In some embodiments, the stable liquid pharmaceutical formulations comprising an anti-PVRIG antibody or antigen binding fragment thereof exhibit IgG purity percentages greater than 94%, greater than 95%, greater than 96%, greater than 97%, or greater than 98%. In some embodiments, the stable liquid pharmaceutical formulations comprising an anti-PVRIG antibody or antigen binding fragment thereof exhibit IgG purity percentages were from about 95% to 98%. In some embodiments, the stable liquid pharmaceutical formulations comprising an anti-PVRIG antibody or antigen binding fragment thereof exhibit IgG purity percentages from about 96% to 97%. In some embodiments, the stable liquid pharmaceutical formulations comprising an anti-PVRIG antibody or antigen binding fragment thereof exhibit HC+LC percentages from about 96% to 100%. In some embodiments, the stable liquid pharmaceutical formulations comprising an anti-PVRIG antibody or antigen binding fragment thereof exhibit HC+LC percentages from about 97% to 100%. In some embodiments, the stable liquid pharmaceutical formulations comprising an anti-PVRIG antibody or antigen binding fragment thereof exhibit HC+LC percentages from about 98% to 100%.

cIEF Analysis

In some embodiments, a capillary isoelectric focusing (cIEF) can be employed to analyze the stable liquid pharmaceutical formulations comprising an anti-PVRIG antibody or antigen binding fragment thereof for the presence of additional species, including for example, minor acidic species.

### MFI Analysis

Antibodies can form sub-visible particles in response to stressed conditions, such as heat, freeze/thaw cycles, and agitation. In some embodiments, a microflow imaging (MFI) analysis can be employed to analyze the stable liquid pharmaceutical formulations comprising an anti-PVRIG antibody or antigen binding fragment thereof for the formation of particles in response to stressed conditions. In some embodiments, the stable liquid pharmaceutical formulations of the anti-PVRIG antibody or antigen binding fragment thereof provide for a formulation capable of stabilizing the anti-PVRIG antibody or antigen binding fragment thereof against these stressed conditions and protecting against the formation of particles. MFI can be used to evaluate particle counts at different size ranges (<2 μm, <5 μm, <10 μm, and <25 μm) in different formulations under stressed conditions. Typically, MFI data can be evaluated to choose an appropriate formulation based on generation of the lowest amount of particles/mL for all sizes of particles across all time points, conditions, and formulations.

### SEC Analysis

In some embodiments, size exclusion chromatography (SEC) can be employed to analyze the stable liquid pharmaceutical formulations comprising an anti-PVRIG antibody or antigen binding fragment thereof. The SEC data showed HMW throughout all time points and conditions; however, it remained stable at about 1%. LMW was present in accelerated conditions and 2-8° C. 8 week time point. Within the 40° C. condition, the LMW did increase from about 1% to 3% from Week 1 to Week 2.

### I. Selected Formulation Embodiments

In some embodiments, the present invention provides a stable liquid pharmaceutical formulation of an anti-PVRIG antibody comprising:
(a) an anti-PVRIG antibody comprising:
   i) a heavy chain variable domain comprising the vhCDR1, vhCDR2, and vhCDR3 from the heavy chain of CHA.7.518.1.H4(S241P) (SEQ ID NO:4), and
   ii) a light chain variable domain comprising the vlCDR1, vlCDR2, and vlCDR3 from the light chain of CHA.7.518.1.H4(S241P) (SEQ ID NO:9); or
   i) a heavy chain variable domain comprising the vhCDR1, vhCDR2, and vhCDR3 from the heavy chain of CHA.7.538.1.H4(S241P) (SEQ ID NO:14), and
   ii) a light chain variable domain comprising the vlCDR1, vlCDR2, and vlCDR3 from the light chain of CHA.7.538.1.H4(S241P) (SEQ ID NO:19);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides a stable liquid pharmaceutical formulation of an anti-PVRIG antibody comprising:
(a) an anti-PVRIG antibody comprising:
   i) a heavy chain variable domain comprising the vhCDR1, vhCDR2, and vhCDR3 from the heavy chain of CHA.7.518.1.H4(S241P) (SEQ ID NO:4), and
   ii) a light chain variable domain comprising the vlCDR1, vlCDR2, and vlCDR3 from the light chain of CHA.7.518.1.H4(S241P) (SEQ ID NO:9); or
   i) a heavy chain variable domain comprising the vhCDR1, vhCDR2, and vhCDR3 from the heavy chain of CHA.7.538.1.H4(S241P) (SEQ ID NO:14), and
   ii) a light chain variable domain comprising the vlCDR1, vlCDR2, and vlCDR3 from the light chain of CHA.7.538.1.H4(S241P) (SEQ ID NO:19).
(a) an anti-PVRIG antibody;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides a stable liquid pharmaceutical formulation comprising:
(a) an anti-PVRIG antibody comprising:
   i) a heavy chain variable domain is from the heavy chain of CHA.7.518.1.H4(S241P) (SEQ ID NO:4), and
   ii) a light chain variable domain is from the light chain of CHA.7.518.1.H4(S241P) (SEQ ID NO:9); or
   i) a heavy chain variable domain is from the heavy chain of CHA.7.538.1.H4(S241P) (SEQ ID NO:14), and
   ii) a light chain variable domain is from the light chain of CHA.7.538.1.H4(S241P) (SEQ ID NO:19);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides a stable liquid pharmaceutical formulation comprising:
(a) an anti-PVRIG antibody comprising:
   i) a heavy chain variable domain is from the heavy chain of CHA.7.518.1.H4(S241P) (SEQ ID NO:4), and
   ii) a light chain variable domain is from the light chain of CHA.7.518.1.H4(S241P) (SEQ ID NO:9); or
   i) a heavy chain variable domain is from the heavy chain of CHA.7.538.1.H4(S241P) (SEQ ID NO:14), and
   ii) a light chain variable domain is from the light chain of CHA.7.538.1.H4(S241P) (SEQ ID NO:19);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides a stable liquid pharmaceutical formulation comprising:
(a) an anti-PVRIG antibody comprising:
   i) a heavy chain comprising:
      a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
   ii) a light chain comprising:
      a) a VL-CL, wherein the VL from CHA.7.518.1.H4(S241P) (SEQ ID NO:9) and wherein the CL region is from human kappa 2 light chain; or
   i) a heavy chain comprising:
      a) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   ii) a light chain comprising:
      a) a VL-CL, wherein the VL from CHA.7.538.1.H4(S241P) (SEQ ID NO:19) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides a stable liquid pharmaceutical formulation comprising:
(a) an anti-PVRIG antibody comprising:
   i) a heavy chain comprising:
      a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
   ii) a light chain comprising:
      a) a VL-CL, wherein the VL from CHA.7.518.1.H4(S241P) (SEQ ID NO:9) and wherein the CL region is from human kappa 2 light chain; or
   i) a heavy chain comprising:
      a) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   ii) a light chain comprising:
      a) a VL-CL, wherein the VL from CHA.7.538.1.H4(S241P) (SEQ ID NO:19) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides a stable liquid pharmaceutical formulation comprising:
(a) an anti-PVRIG antibody comprising:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13); or
   i) a heavy chain comprising the heavy chain from CHA.7.538.1.H4(S241P) (SEQ ID NO:18); and
   ii) a light chain comprising the light chain from CHA.7.538.1.H4(S241P) (SEQ ID NO:23);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides a stable liquid pharmaceutical formulation comprising:
(a) an anti-PVRIG antibody comprising:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13); or
   i) a heavy chain comprising the heavy chain from CHA.7.538.1.H4(S241P) (SEQ ID NO:18); and
   ii) a light chain comprising the light chain from CHA.7.538.1.H4(S241P) (SEQ ID NO:23);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

### VIII. Administration of Formulations of Anti-PVRIG Antibodies

Administration of the pharmaceutical composition comprising anti-PVRIG antibodies of the present invention (e.g., anti-PVRIG antibodies including those with CDRs identical to those shown in FIG. 3), preferably in the form of a sterile aqueous solution, may be done in a variety of ways, As is known in the art, protein therapeutics are often delivered by IV infusion. The antibodies of the present invention may also be delivered using such methods. For example, administration may venious be by intravenous infusion with 0.9% sodium chloride as an infusion vehicle. Such techniques are disclosed in Remington's Pharmaceutical Sciences 16th edition, Osol, A. Ed., 1980.

The dosing amounts and frequencies of administration are, in some embodiments, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. In order to treat a patient or subject, a therapeutically effective dose of the Fc variant of the present invention may be administered. By “therapeutically effective dose” herein is meant a dose that produces the effects for which it is administered.

### IX. Dosing

In some embodiments, the anti-PVRIG antibody and/or antigen binding portion thereof formulations of the present invention can be formulated for administration, including as a unit dosage formulation. In some embodiments, the anti-PVRIG antibody and/or antigen binding portion thereof formulations are administered at a dosage of 0.01 mg/kg of the anti-PVRIG antibody and/or antigen binding portion thereof. In some embodiments, the anti-PVRIG antibody and/or antigen binding portion thereof formulations are administered at a dosage of 0.02 mg/kg of the anti-PVRIG antibody and/or antigen binding portion thereof. In some embodiments, the anti-PVRIG antibody and/or antigen binding portion thereof formulations are administered at a dosage of 0.03 mg/kg of the anti-PVRIG antibody and/or antigen binding portion thereof. In some embodiments, the anti-PVRIG antibody and/or antigen binding portion thereof formulations are administered at a dosage of 0.04 mg/kg of the anti-PVRIG antibody and/or antigen binding portion thereof. In some embodiments, the anti-PVRIG antibody and/or antigen binding portion thereof formulations are administered at a dosage of 0.05 mg/kg of the anti-PVRIG antibody and/or antigen binding portion thereof. In some embodiments, the anti-PVRIG antibody and/or antigen binding portion thereof formulations are administered at a dosage of 0.06 mg/kg of the anti-PVRIG antibody and/or antigen binding portion thereof. In some embodiments, the anti-PVRIG antibody and/or antigen binding portion thereof formulations are administered at a dosage of 0.07 mg/kg of the anti-PVRIG antibody and/or antigen binding portion thereof. In some embodiments, the anti-PVRIG antibody and/or antigen binding portion thereof formulations are administered at a dosage of 0.08 mg/kg of the anti-PVRIG antibody and/or antigen binding portion thereof. In some embodiments, the anti-PVRIG antibody and/or antigen binding portion thereof formulations are administered at a dosage of 0.09 mg/kg of the anti-PVRIG antibody and/or antigen binding portion thereof. In some embodiments, the anti-PVRIG antibody and/or antigen binding portion thereof formulations are administered at a dosage of 0.1 mg/kg of the anti-PVRIG antibody and/or antigen binding portion thereof. In some embodiments, the anti-PVRIG antibody and/or antigen binding portion thereof formulations are administered at a dosage of 0.2 mg/kg of the anti-PVRIG antibody and/or antigen binding portion thereof. In some embodiments, the anti-PVRIG antibody and/or antigen binding portion thereof formulations are administered at a dosage of 0.3 mg/kg of the anti-PVRIG antibody and/or antigen binding portion thereof. In some embodiments, the anti-PVRIG antibody and/or antigen binding portion thereof formulations are administered at a dosage of 0.5 mg/kg of the anti-PVRIG antibody and/or antigen binding portion thereof. In some embodiments, the anti-PVRIG antibody and/or antigen binding portion thereof formulations are administered at a dosage of 0.8 mg/kg of the anti-PVRIG antibody and/or antigen binding portion thereof. In some embodiments, the anti-PVRIG antibody and/or antigen binding portion thereof formulations are administered at a dosage of 1 mg/kg of the anti-PVRIG antibody and/or antigen binding portion thereof. In some embodiments, the anti-PVRIG antibody and/or antigen binding portion thereof formulations are administered at a dosage of 2 mg/kg of the anti-PVRIG antibody and/or antigen binding portion thereof. In some embodiments, the anti-PVRIG antibody and/or antigen binding portion thereof formulations are administered at a dosage of 3 mg/kg of the anti-PVRIG antibody and/or antigen binding portion thereof. In some embodiments, the anti-PVRIG antibody and/or antigen binding portion thereof formulations are administered at a dosage of 4 mg/kg of the anti-PVRIG antibody and/or antigen binding portion thereof. In some embodiments, the anti-PVRIG antibody and/or antigen binding portion thereof formulations are administered at a dosage of 5 mg/kg of the anti-PVRIG antibody and/or antigen binding portion thereof. In some embodiments, the anti-PVRIG antibody and/or antigen binding portion thereof formulations are administered at a dosage of 6 mg/kg of the anti-PVRIG antibody and/or antigen binding portion thereof. In some embodiments, the anti-PVRIG antibody and/or antigen binding portion thereof formulations are administered at a dosage of 7 mg/kg of the anti-PVRIG antibody and/or antigen binding portion thereof. In some embodiments, the anti-PVRIG antibody and/or antigen binding portion thereof formulations are administered at a dosage of 8 mg/kg of the anti-PVRIG antibody and/or antigen binding portion thereof. In some embodiments, the anti-PVRIG antibody and/or antigen binding portion thereof formulations are administered at a dosage of 9 mg/kg of the anti-PVRIG antibody and/or antigen binding portion thereof. In some embodiments, the anti-PVRIG antibody and/or antigen binding portion thereof formulations are administered at a dosage of 10 mg/kg of the anti-PVRIG antibody and/or antigen binding portion thereof. In some embodiments, the anti-PVRIG antibody and/or antigen binding portion thereof formulations are administered at a dosage of 15 mg/kg of the anti-PVRIG antibody and/or antigen binding portion thereof. In some embodiments, the anti-PVRIG antibody and/or antigen binding portion thereof formulations are administered at a dosage of 20 mg/kg of the anti-PVRIG antibody and/or antigen binding portion thereof.

In some embodiments, the anti-PVRIG antibody and/or antigen binding portion thereof formulations is administered at a dosage of about 0.01 mg/kg to about 20 mg/kg of the anti-PVRIG antibody. In some embodiments, the anti-PVRIG antibody and/or antigen binding portion thereof formulations is administered at a dosage of about 0.01 mg/kg to about 10 mg/kg of the anti-PVRIG antibody. In some embodiments, the anti-PVRIG antibody and/or antigen binding portion thereof formulations is administered at a dosage of about 20 mg/kg. In some embodiments, the anti-PVRIG antibody and/or antigen binding portion thereof formulations is administered at a dosage of about 20 mg/kg each 4 weeks. In some embodiments, the anti-PVRIG antibody and/or antigen binding portion thereof formulations is administered at a dosage of about 20 mg/kg IV each 4 weeks. In some embodiments, formulation is administered at a dosage of about 0.1 mg/kg to about 10 mg/kg of the anti-PVRIG antibody. In some embodiments, formulation is administered at a dosage of about 1 mg/kg to about 10 mg/kg of the anti-PVRIG antibody. In some embodiments, formulation is administered at a dosage of about 2 mg/kg to about 10 mg/kg of the anti-PVRIG antibody. In some embodiments, formulation is administered at a dosage of about 3 mg/kg to about 10 mg/kg of the anti-PVRIG antibody. In some embodiments, formulation is administered at a dosage of about 4 mg/kg to about 10 mg/kg of the anti-PVRIG antibody. In some embodiments, formulation is administered at a dosage of about 5 mg/kg to about 10 mg/kg of the anti-PVRIG antibody. In some embodiments, formulation is administered at a dosage of about 5 mg/kg to about 10 mg/kg of the anti-PVRIG antibody. In some embodiments, formulation is administered at a dosage of about 7 mg/kg to about 10 mg/kg of the anti-PVRIG antibody. In some embodiments, formulation is administered at a dosage of about 8 mg/kg to about 10 mg/kg of the anti-PVRIG antibody. In some embodiments, formulation is administered at a dosage of about 9 mg/kg to about 10 mg/kg of the anti-PVRIG antibody. In some embodiments, formulation is administered at a dosage of about 15 mg/kg of the anti-PVRIG antibody. In some embodiments, formulation is administered at a dosage of about 20 mg/kg of the anti-PVRIG antibody. In some embodiments, formulation is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg of the anti-PVRIG antibody.

A. Selected Dosing with Formulation Embodiments

In some embodiments, the present invention provides for administration of a stable liquid pharmaceutical formulation of an anti-PVRIG antibody in combination with an anti-TIGIT antibody and pembrolizumab, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4(S241P) (SEQ ID NO:4), and
   ii) a light chain variable domain comprising the vlCDR1, vlCDR2, and vlCDR3 from the light chain of CHA.7.518.1.H4(S241P) (SEQ ID NO:9); or
   i) a heavy chain variable domain comprising the vhCDR1, vhCDR2, and vhCDR3 from the heavy chain of CHA.7.538.1.2.H4(S241P) (SEQ ID NO:14), and
   ii) a light chain variable domain comprising the vlCDR1, vlCDR2, and vlCDR3 from the light chain of CHA.7.538.1.2.H4(S241P) (SEQ ID NO:19);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for administration of a stable liquid pharmaceutical formulation of an anti-PVRIG antibody in combination with an anti-TIGIT antibody and pembrolizumab, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4(S241P) (SEQ ID NO:4), and
   ii) a light chain variable domain comprising the vlCDR1, vlCDR2, and vlCDR3 from the light chain of CHA.7.518.1.H4(S241P) (SEQ ID NO:9); or
   i) a heavy chain variable domain comprising the vhCDR1, vhCDR2, and vhCDR3 from the heavy chain of CHA.7.538.1.2.H4(S241P) (SEQ ID NO:14), and
   ii) a light chain variable domain comprising the vlCDR1, vlCDR2, and vlCDR3 from the light chain of CHA.7.538.1.2.H4(S241P) (SEQ ID NO:19);
(a) an anti-PVRIG antibody;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for administration of a stable liquid pharmaceutical formulation of an anti-PVRIG antibody in combination with an anti-TIGIT antibody and pembrolizumab, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain is from the heavy chain of CHA.7.518.1.H4(S241P) (SEQ ID NO:4), and
   ii) a light chain variable domain is from the light chain of CHA.7.518.1.H4(S241P) (SEQ ID NO:9); or
   i) a heavy chain variable domain is from the heavy chain of CHA.7.538.1.H4(S241P) (SEQ ID NO:14), and
   ii) a light chain variable domain is from the light chain of CHA.7.538.1.H4(S241P) (SEQ ID NO:19);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for administration of a stable liquid pharmaceutical formulation of an anti-PVRIG antibody in combination with an anti-TIGIT antibody and pembrolizumab, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain is from the heavy chain of CHA.7.518.1.H4(S241P) (SEQ ID NO:4), and
   ii) a light chain variable domain is from the light chain of CHA.7.518.1.H4(S241P) (SEQ ID NO:9); or
   i) a heavy chain variable domain is from the heavy chain of CHA.7.538.1.H4(S241P) (SEQ ID NO:14), and
   ii) a light chain variable domain is from the light chain of CHA.7.538.1.H4(S241P) (SEQ ID NO:19);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for administration of a stable liquid pharmaceutical formulation of an anti-PVRIG antibody in combination with an anti-TIGIT antibody and pembrolizumab, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
   ii) a light chain comprising:
      a) a VL-CL, wherein the VL from CHA.7.518.1.H4(S241P) (SEQ ID NO:9) and wherein the CL region is from human kappa 2 light chain; or
   i) a heavy chain comprising:
      a) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   ii) a light chain comprising:
      a) a VL-CL, wherein the VL from CHA.7.538.1.H4(S241P) (SEQ ID NO:19) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for administration of a stable liquid pharmaceutical formulation of an anti-PVRIG antibody in combination with an anti-TIGIT antibody and pembrolizumab, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
   ii) a light chain comprising:
      a) a VL-CL, wherein the VL from CHA.7.518.1.H4(S241P) (SEQ ID NO:9) and wherein the CL region is from human kappa 2 light chain; or
   i) a heavy chain comprising:
      a) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   ii) a light chain comprising:
      a) a VL-CL, wherein the VL from CHA.7.538.1.H4(S241P) (SEQ ID NO:19) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments, the present invention provides for administration of a stable liquid pharmaceutical formulation of an anti-PVRIG antibody in combination with an anti-TIGIT antibody and pembrolizumab, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13); or
   i) a heavy chain comprising the heavy chain from CHA.7.538.1.H4(S241P) (SEQ ID NO:18); and
   ii) a light chain comprising the light chain from CHA.7.538.1.H4(S241P) (SEQ ID NO:23);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0.

In some embodiments, the present invention provides for administration of a stable liquid pharmaceutical formulation of an anti-PVRIG antibody in combination with an anti-TIGIT antibody and pembrolizumab, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2.

In some embodiments of the stable liquid pharmaceutical formulation, the formulation is administered with pembrolizumab. In some embodiments of the stable liquid pharmaceutical formulation, the formulation is administered with pembrolizumab. In some embodiments of the stable liquid pharmaceutical formulation, the formulation is administered with pembrolizumab, as shown in FIG. 5.

### X. Methods of Using the Anti-PVRIG Antibody Formulations

### A. Therapeutic Uses

The anti-PVRIG antibodies (e.g., anti-PVRIG antibodies including those with CDRs identical to those shown in FIG. 3) find use in treating patients or subjects, such as human subjects, generally with a condition associated with PVRIG. The term “treatment” as used herein, refers to both therapeutic treatment, maintenance treatment, and prophylactic or preventative measures that cure, slow down, lessen symptoms of, and/or halt progression of a diagnosed pathologic condition or disorder, which in this example relates to treatment of cancer; however, also as described below, uses of antibodies and pharmaceutical compositions are also provided for treatment of infectious disease, sepsis, and/or autoimmune conditions, and/or for inhibiting an undesirable immune activation that follows gene therapy. Those in need of treatment include those already with cancer, those suspected of having cancer, as well as those in which the cancer is to be prevented. Hence, the mammal to be treated herein may have been diagnosed as having the cancer or may be predisposed or susceptible to the cancer. As used herein the term “treating” refers to preventing, delaying the onset of, curing, reversing, attenuating, alleviating, minimizing, suppressing, halting the deleterious effects or stabilizing of discernible symptoms of the above-described cancerous diseases, disorders or conditions. In some aspects, a subject is successfully “treated” for cancer according to the methods of the present disclosure if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof. It also includes managing the cancer or maintaining treatment as described above. By “manage” or “maintain” or “maintenance” it is meant reducing the severity of the disease, reducing the frequency of episodes of the disease, reducing the duration of such episodes, reducing the severity of such episodes, slowing/reducing cancer cell growth or proliferation, slowing progression of at least one symptom, amelioration of at least one measurable physical parameter and the like. For example, immunostimulatory anti-PVRIG immune molecules should promote T cell or NK or cytokine immunity against target cells, e.g., cancer, infected or pathogen cells and thereby treat cancer or infectious diseases by depleting the cells involved in the disease condition. Conversely, immunoinhibitory anti-PVRIG immune molecules should reduce T cell or NK activity and/or or the secretion of proinflammatory cytokines which are involved in the disease pathology of some immune disease such as autoimmune, inflammatory or allergic conditions and thereby treat or ameliorate the disease pathology and tissue destruction that may be associated with such conditions (e.g., joint destruction associated with rheumatoid arthritis conditions).

The PVRIG antibodies of the invention, the TIGIT antibodies of the invention, and pembrolizumab are provided in therapeutically effective dosages. A “therapeutically effective dosage” of an anti-PVRIG and or anti-TIGIT immune molecule according to at least some embodiments of the present invention preferably results in a decrease in severity of disease symptoms, an increase in frequency and duration of disease symptom-free periods, an increase in lifespan, disease remission, or a prevention or reduction of impairment or disability due to the disease affliction. For example, for the treatment of tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject.

### 1. Level of Serum IFNγ

In some embodiments, the “therapeutically effective dosage” of the anti-PVRIG antibody of the invention, the anti-TIGIT antibody of the invention, and pembrolizumab according to at least some embodiments of the present invention increases the level of serum IFNγ in a subject relative to untreated subjects and/or subjects prior to treatment. In some embodiments, the level of serum IFNγ in the subject treated with the therapeutically effective dosage of the anti-PVRIG antibody, the anti-TIGIT antibody, and pembrolizumab is increased by at least 1-fold, 2-fold, 3-fold, 4-fold, 5-fold, 6-fold, 7-fold, 8-fold, 9-fold, 10-fold, or more, relative to untreated subjects. In some embodiments, the level of serum IFNγ in the subject treated with the therapeutically effective dosage of the anti-PVRIG antibody, the anti-TIGIT antibody, and pembrolizumab is increased by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1100%, 1200%, 1300%, 1400%, 1500% or more, relative to untreated subjects. In some embodiments, the level of serum IFNγ in the subject is increased by at least 200%, 300%, 400%, or more. In some embodiments, the level of serum IFNγ in the subject is increased by at least 1000%, 1100% 1200%, 1300%, 1400%, 1500% or more. In some embodiments, the level of serum IFNγ in the subject is increased by at least 1200%, or more. In some embodiments, the level of serum IFNγ in the subject is increased by at least 1300%, or more. In some embodiments, the level of serum IFNγ in the subject is increased by at least 1400%, or more. In some embodiments, the level of serum IFNγ in the subject is increased by at least 1500%, or more.

In some embodiments, the subject treated with the therapeutically effective dosage of the anti-PVRIG antibody, the anti-TIGIT antibody, and pembrolizumab exhibits an increased level of serum IFNγ of at least 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 110 pg/mL, 120 pg/mL, 130 pg/mL, 140 pg/mL, 150 pg/mL, 160 pg/mL, 170 pg/mL, 180 pg/mL, 190 pg/mL, 200 pg/mL, 210 pg/mL, 220 pg/mL, 230 pg/mL, 240 pg/mL, 250 pg/mL, 260 pg/mL, 270 pg/mL, 280 pg/mL, 290 pg/mL, 300 pg/mL, 310 pg/mL, 320 pg/mL, 330 pg/mL, 340 pg/mL, 350 pg/mL, 360 pg/mL, 370 pg/mL, 380 pg/mL, 390 pg/mL, 400 pg/mL, 410 pg/mL, 420 pg/mL, 430 pg/mL, 440 pg/mL, 450 pg/mL, 460 pg/mL, 470 pg/mL, 480 pg/mL, 490 pg/mL, 500 pg/mL, or more. In some embodiments, the subject treated with the therapeutically effective dosage of the anti-PVRIG antibody, the anti-TIGIT antibody, and pembrolizumab exhibits an increased level of serum IFNγ of at least 20 pg/mL, 30 pg/mL, 40 pg/mL, or more. In some embodiments, the subject treated with the therapeutically effective dosage of the anti-PVRIG antibody, the anti-TIGIT antibody, and pembrolizumab exhibits an increased level of serum IFNγ of at least 40 pg/mL, 50 pg/mL, 60 pg/mL, 70 pg/mL, 80 pg/mL or more. In some embodiments, the subject treated with the therapeutically effective dosage of the anti-PVRIG antibody, the anti-TIGIT antibody, and pembrolizumab exhibits an increased level of serum IFNγ of at least 70 pg/mL, or more. In some embodiments, the subject treated with the therapeutically effective dosage of the anti-PVRIG antibody, the anti-TIGIT antibody, and pembrolizumab exhibits an increased level of serum IFNγ of at least 80 pg/mL, or more.

### 2. Level of Serum IL-6

In some embodiments, the “therapeutically effective dosage” of the anti-PVRIG antibody of the invention, the anti-TIGIT antibody of the invention, and pembrolizumab according to at least some embodiments of the present invention decreases the level of serum IL-6 in a subject relative to untreated subjects, or non-responsive subjects. In some embodiments, the level of serum IL-6 in the subject treated with the therapeutically effective dosage of the anti-PVRIG antibody, the anti-TIGIT antibody, and pembrolizumab is decreased by at least about 10%, 20%, 30%, 40%, 50%, 60%, 70%, 80%, or 90% relative to untreated subjects, or non-responsive subjects. In some embodiments, the “therapeutically effective dosage” of the anti-PVRIG antibody of the invention, the anti-TIGIT antibody of the invention, and pembrolizumab according to at least some embodiments of the present invention is predicted according to the baseline level of serum IL-6 in a subject. In some embodiments, the baseline level of serum IL-6 in the subject is based on a preset reference value. In some embodiments, the baseline level of serum IL-6 in the subject treated with the therapeutically effective dosage of the anti-PVRIG antibody, the anti-TIGIT antibody, and pembrolizumab is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally no more than about 1 pg/mL, 2 pg/mL, or 3 pg/mL. In some embodiments, the preset reference value for the level of serum IL-6 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally no more than about 1 pg/mL, 2 pg/mL, or 3 pg/mL.

In some embodiments, the subject treated with the therapeutically effective dosage of the anti-PVRIG antibody, the anti-TIGIT antibody, and pembrolizumab exhibits a decreased and/or lower baseline serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally the subject exhibits a decreased and/or lower baseline serum level of IL-6 of no more than about 1 pg/mL, 2 pg/mL, or 3 pg/mL. In some embodiments, the subject treated with the therapeutically effective dosage of the anti-PVRIG antibody, the anti-TIGIT antibody, and pembrolizumab exhibits a decreased and/or lower baseline serum level of IL-6 of no more than about 1 pg/mL. In some embodiments, the subject treated with the therapeutically effective dosage of the anti-PVRIG antibody, the anti-TIGIT antibody, and pembrolizumab exhibits a decreased and/or lower baseline serum level of IL-6 of no more than about 2 pg/mL. In some embodiments, the subject treated with the therapeutically effective dosage of the anti-PVRIG antibody, the anti-TIGIT antibody, and pembrolizumab exhibits a decreased and/or lower baseline serum level of IL-6 of no more than about 3 pg/mL. In some embodiments, the subject treated with the therapeutically effective dosage of the anti-PVRIG antibody, the anti-TIGIT antibody, and pembrolizumab exhibits a decreased and/or lower baseline serum level of IL-6 of no more than about 4 pg/mL. In some embodiments, the subject treated with the therapeutically effective dosage of the anti-PVRIG antibody, the anti-TIGIT antibody, and pembrolizumab exhibits a decreased and/or lower baseline serum level of IL-6 of no more than about 5 pg/mL. In some embodiments, the subject treated with the therapeutically effective dosage of the anti-PVRIG antibody, the anti-TIGIT antibody, and pembrolizumab exhibits a decreased and/or lower baseline serum level of IL-6 of no more than about 6 pg/mL. In some embodiments, the subject treated with the therapeutically effective dosage of the anti-PVRIG antibody, the anti-TIGIT antibody, and pembrolizumab exhibits a lower baseline serum level of IL-6 of no more than about 1 pg/mL. In some embodiments, the subject treated with the therapeutically effective dosage of the anti-PVRIG antibody, the anti-TIGIT antibody, and pembrolizumab exhibits a lower baseline serum level of IL-6 of no more than about 2 pg/mL. In some embodiments, the subject treated with the therapeutically effective dosage of the anti-PVRIG antibody, the anti-TIGIT antibody, and pembrolizumab exhibits a lower baseline serum level of IL-6 of no more than about 3 pg/mL. In some embodiments, the subject treated with the therapeutically effective dosage of the anti-PVRIG antibody, the anti-TIGIT antibody, and pembrolizumab exhibits a lower baseline serum level of IL-6 of no more than about 4 pg/mL. In some embodiments, the subject treated with the therapeutically effective dosage of the anti-PVRIG antibody, the anti-TIGIT antibody, and pembrolizumab exhibits a lower baseline serum level of IL-6 of no more than about 5 pg/mL. In some embodiments, the subject treated with the therapeutically effective dosage of the anti-PVRIG antibody, the anti-TIGIT antibody, and pembrolizumab exhibits a decreased and/or lower baseline serum level of IL-6 of no more than about 6 pg/mL.

In some embodiments, the subject treated with the therapeutically effective dosage of the anti-PVRIG antibody, the anti-TIGIT antibody, and pembrolizumab exhibits a decreased and/or lower baseline serum level of IL-6 for at least about 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, or 40 weeks. In some embodiments, the subject treated with the therapeutically effective dosage of the anti-PVRIG antibody, the anti-TIGIT antibody, and pembrolizumab exhibits a decreased and/or lower baseline serum level of IL-6 for at least about 24 weeks.

In some embodiments, the serum level of IL-6 can be employed as a biomarker or companion diagnostic for use in determining treatment regimens as well as predicting or determining treatment efficacy. In some embodiments, the serum level of IL-6 can be employed as a biomarker or companion diagnostic for use in the determining treatment regimens as well as predicting or determining treatment efficacy for treatment with the anti-PVRIG antibody of the invention, the anti-TIGIT antibody of the invention, and pembrolizumab. In some embodiments, the serum level of IL-6 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally no more than about 1 pg/mL, 2 pg/mL, or 3 pg/mL. In some embodiments, a serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally no more than about 1 pg/mL, 2 pg/mL, or 3 pg/mL, when employed as a biomarker or companion diagnostic for use in the determining treatment regimens as well as predicting or determining treatment efficacy for treatment with the anti-PVRIG antibody of the invention, the anti-TIGIT antibody of the invention, and pembrolizumab. In some embodiments, the serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally no more than about 1 pg/mL, 2 pg/mL, or 3 pg/mL. In some embodiments, a serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally no more than about 1 pg/mL, 2 pg/mL, or 3 pg/mL is predictive of treatment efficacy.

In some embodiments, there is an increased prediction of a response to the combination of the anti-PVRIG antibody of the invention, the anti-TIGIT antibody of the invention, and pembrolizumab according to at least some embodiments of the present invention when the decreased and/or lower baseline serum level IL-6 as compared to normal level (such as the serum IL-6 level of an untreated subject or non-responsive subject or as compared to a preset reference value) is detected. In some embodiments, the serum level of IL-6 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally no more than about 1 pg/mL, 2 pg/mL, or 3 pg/mL. In some embodiments, a serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally no more than about 1 pg/mL, 2 pg/mL, or 3 pg/mL is indicative of an increased prediction of a response to the combination of the anti-PVRIG antibody of the invention, the anti-TIGIT antibody of the invention, and pembrolizumab.

In some embodiments, there is an increased prediction of a response to the combination of the anti-PVRIG antibody of the invention, the anti-TIGIT antibody of the invention, and pembrolizumab when the subject exhibits a lower baseline serum level of IL-6 as compared to a preset reference value. In some embodiments, the lower baseline serum level of IL-6 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL. In some embodiments, the preset reference level of IL-6 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL. In some embodiments, there is an increased prediction of a response to the combination of the anti-PVRIG antibody of the invention, the anti-TIGIT antibody of the invention, and pembrolizumab when the subject exhibits a lower baseline serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL. In some embodiments, there is an increased prediction of a response to the combination of the anti-PVRIG antibody of the invention, the anti-TIGIT antibody of the invention, and pembrolizumab when the subject exhibits a lower baseline serum level of IL-6 of no more than about 1 pg/mL, 2 pg/mL, or 3 pg/mL. In some embodiments, the subject treated with the therapeutically effective dosage of the anti-PVRIG antibody, the anti-TIGIT antibody, and pembrolizumab exhibits a lower baseline serum level of IL-6 of no more than about 1 pg/mL. In some embodiments, the subject treated with the therapeutically effective dosage of the anti-PVRIG antibody, the anti-TIGIT antibody, and pembrolizumab exhibits a lower baseline serum level of IL-6 of no more than about 2 pg/mL. In some embodiments, the subject treated with the therapeutically effective dosage of the anti-PVRIG antibody, the anti-TIGIT antibody, and pembrolizumab exhibits a lower baseline serum level of IL-6 of no more than about 3 pg/mL. In some embodiments, the subject treated with the therapeutically effective dosage of the anti-PVRIG antibody, the anti-TIGIT antibody, and pembrolizumab exhibits a lower baseline serum level of IL-6 of no more than about 4 pg/mL. In some embodiments, the subject treated with the therapeutically effective dosage of the anti-PVRIG antibody, the anti-TIGIT antibody, and pembrolizumab exhibits a lower baseline serum level of IL-6 of no more than about 5 pg/mL. In some embodiments, the subject treated with the therapeutically effective dosage of the anti-PVRIG antibody, the anti-TIGIT antibody, and pembrolizumab exhibits a decreased and/or lower baseline serum level of IL-6 of no more than about 6 pg/mL.

In some embodiments, the invention provides a method for determining a cancer patient population for treatment with the anti-PVRIG antibody of the invention, the anti-TIGIT antibody of the invention, and pembrolizumab the method comprising:
(a) detecting the presence of IL-6 in the serum from the cancer patient,
(b) quantitating the measurement of the level of IL-6;
(c) treating the cancer patient with the anti-PVRIG antibody of the invention, the anti-TIGIT antibody of the invention, and pembrolizumab when IL-6 is present at a decreased and/or lower baseline serum level as compared to a control or a patient that is not treated with or non-responsive to the combination.

In some embodiments, the invention provides a method for determining a cancer patient population for treatment with the anti-PVRIG antibody of the invention, the anti-TIGIT antibody of the invention, and pembrolizumab the method comprising:
(a) detecting the presence of IL-6 in the serum from the cancer patient,
(b) quantitating the measurement of the level of IL-6;
(c) treating the cancer patient with the anti-PVRIG antibody of the invention, the anti-TIGIT antibody of the invention, and pembrolizumab when IL-6 is present at a lower baseline serum level. In some embodiments, lower baseline serum level of IL-6 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL. In some embodiments, the lower baseline serum level of IL-6 is no more than about 1 pg/mL, 2 pg/mL, or 3 pg/mL. In some embodiments, the lower baseline serum level of IL-6 is no more than about 1 pg/mL. In some embodiments, the lower baseline serum level of IL-6 is no more than about 2 pg/mL. In some embodiments, the lower baseline serum level of IL-6 is no more than about 3 pg/mL.

In some embodiments, cancer patients having a decreased and/or lower baseline serum level of IL-6 of at least about 10%, 20%, 30%, 40%, 50%, 60%, 70%, 80%, or 90% relative to an untreated subject, or a non-responsive subject has a higher probability to respond to the combination of the anti-PVRIG antibody of the invention, the anti-TIGIT antibody of the invention, and pembrolizumab. In some embodiments, the anti-PVRIG antibody comprises the antibody sequence in FIG. 3, including for example, CHA.7.518.1.H4(S241P)vhCDR1, CHA.7.518.1.H4(S241P)vhCDR2, CHA.7.518.1.H4(S241P)vhCDR3, CHA.7.518.1.H4(S241P)vlCDR1, CHA.7.518.1.H4(S241P)vlCDR2, and CHA.7.518.1.H4(S241P)vlCDR3.

In some embodiments, cancer patients having a decreased and/or lower baseline serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally having a serum level of IL-6 of no more than about 1 pg/mL, 2 pg/mL, or 3 pg/mL, have a higher probability to respond to the combination of the anti-PVRIG antibody of the invention, the anti-TIGIT antibody of the invention, and pembrolizumab, relative to cancer patients with a higher serum level of IL-6. In some embodiments, the anti-PVRIG antibody comprises the antibody sequence in FIG. 3, including for example, CHA.7.518.1.H4(S241P)vhCDR1, CHA.7.518.1.H4(S241P)vhCDR2, CHA.7.518.1.H4(S241P)vhCDR3, CHA.7.518.1.H4(S241P)vlCDR1, CHA.7.518.1.H4(S241P)vlCDR2, and CHA.7.518.1.H4(S241P)vlCDR3.

In some embodiments, cancer patients having a lower baseline serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL have a higher probability to respond to the combination of the anti-PVRIG antibody of the invention, the anti-TIGIT antibody of the invention, and pembrolizumab. In some embodiments, cancer patients having a lower baseline serum level of IL-6 of no more than about 1 pg/mL, 2 pg/mL, or 3 pg/mL, have a higher probability to respond to the combination of the anti-PVRIG antibody of the invention, the anti-TIGIT antibody of the invention, and pembrolizumab, relative to cancer patients with a higher serum level of IL-6. In some embodiments, the anti-PVRIG antibody comprises the antibody sequence in FIG. 3, including for example, CHA.7.518.1.H4(S241P)vhCDR1, CHA.7.518.1.H4(S241P)vhCDR2, CHA.7.518.1.H4(S241P)vhCDR3, CHA.7.518.1.H4(S241P)vlCDR1, CHA.7.518.1.H4(S241P)vlCDR2, and CHA.7.518.1.H4(S241P)vlCDR3.

In some embodiments, cancer patients having a decreased and/or lower baseline serum level of IL-6 of no more than about 1 pg/mL have a higher probability to respond to the combination of the anti-PVRIG antibody of the invention, the anti-TIGIT antibody of the invention, and pembrolizumab, relative to cancer patients with a higher serum level of IL-6. In some embodiments, cancer patients having a decreased and/or lower baseline serum level of IL-6 of no more than about 2 pg/mL have a higher probability to respond to the combination of the anti-PVRIG antibody of the invention, the anti-TIGIT antibody of the invention, and pembrolizumab, relative to cancer patients with a higher serum level of IL-6. In some embodiments, cancer patients having a decreased and/or lower baseline serum level of IL-6 of no more than about 3 pg/mL, have a higher probability to respond to the combination of the anti-PVRIG antibody of the invention, the anti-TIGIT antibody of the invention, and pembrolizumab, relative to cancer patients with a higher serum level of IL-6. In some embodiments, cancer patients having a decreased and/or lower baseline serum level of IL-6 of no more than about 4 pg/mL, have a higher probability to respond to the combination of the anti-PVRIG antibody of the invention, the anti-TIGIT antibody of the invention, and pembrolizumab, relative to cancer patients with a higher serum level of IL-6. In some embodiments, cancer patients having a decreased and/or lower baseline serum level of IL-6 of no more than about 15 pg/mL, have a higher probability to respond to the combination of the anti-PVRIG antibody of the invention, the anti-TIGIT antibody of the invention, and pembrolizumab, relative to cancer patients with a higher serum level of IL-6.

In some embodiments, cancer patients having a lower baseline serum level of IL-6 of no more than about 1 pg/mL have a higher probability to respond to the combination of the anti-PVRIG antibody of the invention, the anti-TIGIT antibody of the invention, and pembrolizumab, relative to cancer patients with a higher serum level of IL-6. In some embodiments, cancer patients having a lower baseline serum level of IL-6 of no more than about 2 pg/mL have a higher probability to respond to the combination of the anti-PVRIG antibody of the invention, the anti-TIGIT antibody of the invention, and pembrolizumab, relative to cancer patients with a higher serum level of IL-6. In some embodiments, cancer patients having a decreased and/or lower baseline serum level of IL-6 of no more than about 3 pg/mL have a higher probability to respond to the combination of the anti-PVRIG antibody of the invention, the anti-TIGIT antibody of the invention, and pembrolizumab, relative to cancer patients with a higher serum level of IL-6. In some embodiments, cancer patients having a decreased and/or lower baseline serum level of IL-6 of no more than about 4 pg/mL have a higher probability to respond to the combination of the anti-PVRIG antibody of the invention, the anti-TIGIT antibody of the invention, and pembrolizumab, relative to cancer patients with a higher serum level of IL-6. In some embodiments, cancer patients having a decreased and/or lower baseline serum level of IL-6 of no more than about 15 pg/mL have a higher probability to respond to the combination of the anti-PVRIG antibody of the invention, the anti-TIGIT antibody of the invention, and pembrolizumab, relative to cancer patients with a higher serum level of IL-6.

In some embodiments, cancer patients, the invention provides a method for predicting or determining the efficacy of treatment with the anti-PVRIG antibody of the invention, the anti-TIGIT antibody of the invention, and pembrolizumab, the method comprising:
(a) measuring the serum level of IL-6 in the cancer patient;
(b) quantitating the measurement of the serum level of IL-6;
(c) correlating the serum level of IL-6 with the efficacy of treatment, wherein IL-6 at a decreased and/or lower baseline serum level as compared to a control or a patient that is not treated with or non-responsive to the combination of the anti-PVRIG antibody of the invention, the anti-TIGIT antibody of the invention, and pembrolizumab, is indicative of treatment efficacy.

In some embodiments, the method further comprises (d) optionally treating the cancer patient with the anti-PVRIG antibody of the invention, the anti-TIGIT antibody of the invention, and pembrolizumab when IL-6 in (a) is present at a decreased and/or lower baseline serum level as compared to a control or a patient that is not treated with or non-responsive to the treatment.

In some embodiments, cancer patients, the invention provides a method for predicting or determining the efficacy of treatment with the anti-PVRIG antibody of the invention, the anti-TIGIT antibody of the invention, and pembrolizumab, the method comprising:
(a) measuring the serum level of IL-6 in the cancer patient;
(b) quantitating the measurement of the serum level of IL-6;
(c) correlating the serum level of IL-6 with the efficacy of treatment, wherein IL-6 at a lower baseline serum level is indicative of treatment efficacy.

In some embodiments, the method further comprises (d) optionally treating the cancer patient with the anti-PVRIG antibody of the invention, the anti-TIGIT antibody of the invention, and pembrolizumab when IL-6 in (a) is present at a lower baseline serum level as compared to a preset reference value. In some embodiments, the lower baseline serum level of IL-6 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL. In some embodiments, the lower baseline serum level of IL-6 is no more than about 1 pg/mL, 2 pg/mL, or 3 pg/mL. In some embodiments, the lower baseline serum level of IL-6 is no more than about 1 pg/mL. In some embodiments, the lower baseline serum level of IL-6 is no more than about 2 pg/mL. In some embodiments, the lower baseline serum level of IL-6 is no more than about 3 pg/mL.

In some embodiments, a decreased and/or lower baseline serum level of IL-6 of at least about 10%, 20%, 30%, 40%, 50%, 60%, 70%, 80%, or 90% relative to an untreated subject, or a non-responsive subject is indicative of efficacy of treatment with the anti-PVRIG antibody of the invention, the anti-TIGIT antibody of the invention, and pembrolizumab.

In some embodiments, a decreased and/or lower baseline serum level of IL-6 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, or 15 pg/mL, optionally no more than about 1 pg/mL, 2 pg/mL, or 3 pg/mL, is indicative of efficacy of treatment with the anti-PVRIG antibody of the invention, the anti-TIGIT antibody of the invention, and pembrolizumab. In some embodiments, a decreased and/or lower baseline serum level of IL-6 of no more than about 1 pg/mL, 2 pg/mL, or 3 pg/mL, is indicative of efficacy of treatment with the anti-PVRIG antibody of the invention, the anti-TIGIT antibody of the invention, and pembrolizumab. In some embodiments, a decreased and/or lower baseline serum level of IL-6 of no more than about 1 pg/mL, is indicative of efficacy of treatment with the anti-PVRIG antibody of the invention, the anti-TIGIT antibody of the invention, and pembrolizumab. In some embodiments, a decreased and/or lower baseline serum level of IL-6 of no more than about 2 pg/mL, is indicative of efficacy of treatment with the anti-PVRIG antibody of the invention, the anti-TIGIT antibody of the invention, and pembrolizumab. In some embodiments, a decreased and/or lower baseline serum level of IL-6 of no more than about 3 pg/mL, is indicative of efficacy of treatment with the anti-PVRIG antibody of the invention, the anti-TIGIT antibody of the invention, and pembrolizumab.

In some embodiments, a decreased and/or lower baseline serum level of IL-6 of no more than about 1 pg/mL is indicative of efficacy of treatment with the anti-PVRIG antibody of the invention, the anti-TIGIT antibody of the invention, and pembrolizumab. In some embodiments, a decreased and/or lower baseline serum level of IL-6 of no more than about 2 pg/mL is indicative of efficacy of treatment with the anti-PVRIG antibody of the invention, the anti-TIGIT antibody of the invention, and pembrolizumab. In some embodiments, a decreased and/or lower baseline serum level of IL-6 of no more than about 3 pg/mL is indicative of efficacy of treatment with the anti-PVRIG antibody of the invention, the anti-TIGIT antibody of the invention, and pembrolizumab. In some embodiments, a decreased and/or lower baseline serum level of IL-6 of no more than about 4 pg/mL is indicative of efficacy of treatment with the anti-PVRIG antibody of the invention, the anti-TIGIT antibody of the invention, and pembrolizumab. In some embodiments, a decreased and/or lower baseline serum level of IL-6 of no more than about 5 pg/mL is indicative of efficacy of treatment with the anti-PVRIG antibody of the invention, the anti-TIGIT antibody of the invention, and pembrolizumab. In some embodiments, a decreased and/or lower baseline serum level of IL-6 of no more than about 6 pg/mL is indicative of efficacy of treatment with the anti-PVRIG antibody of the invention, the anti-TIGIT antibody of the invention, and pembrolizumab.

### 3. Level of Serum IL-8

In some embodiments, the “therapeutically effective dosage” of the anti-PVRIG antibody of the invention, the anti-TIGIT antibody of the invention, and pembrolizumab according to at least some embodiments of the present invention decreases the level of serum IL-8 in a subject relative to untreated subjects, or non-responsive subjects. In some embodiments, the level of serum IL-8 in the subject treated with the therapeutically effective dosage of the anti-PVRIG antibody, the anti-TIGIT antibody, and pembrolizumab is decreased by at least about 10%, 20%, 30%, 40%, 50%, 60%, 70%, 80%, or 90% relative to untreated subjects, or non-responsive subjects. In some embodiments, the “therapeutically effective dosage” of the anti-PVRIG antibody of the invention, the anti-TIGIT antibody of the invention, and pembrolizumab according to at least some embodiments of the present invention is predicted according to the baseline level of serum IL-8 in a subject. In some embodiments, the baseline level of serum IL-8 in the subject is based on a preset reference value. In some embodiments, the baseline level of serum IL-8 in the subject treated with the therapeutically effective dosage of the anti-PVRIG antibody, the anti-TIGIT antibody, and pembrolizumab is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the baseline serum level of IL-8 is no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the baseline serum level of IL-8 is no more than about 25 pg/mL.

In some embodiments, the subject treated with the therapeutically effective dosage of the anti-PVRIG antibody, the anti-TIGIT antibody, and pembrolizumab exhibits a decreased and/or lower baseline serum level of IL-8 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the subject exhibits a decreased and/or lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the baseline serum level of IL-8 is no more than about 25 pg/mL. In some embodiments, the subject treated with the therapeutically effective dosage of the anti-PVRIG antibody, the anti-TIGIT antibody, and pembrolizumab exhibits a decreased and/or lower baseline serum level of IL-8 of no more than about 30 pg/mL. In some embodiments, the subject treated with the therapeutically effective dosage of the anti-PVRIG antibody, the anti-TIGIT antibody, and pembrolizumab exhibits a decreased and/or lower baseline serum level of IL-8 of no more than about 25 pg/mL. In some embodiments, the subject treated with the therapeutically effective dosage of the anti-PVRIG antibody, the anti-TIGIT antibody, and pembrolizumab exhibits a decreased and/or lower baseline serum level of IL-8 of no more than about 20 pg/mL. In some embodiments, the subject treated with the therapeutically effective dosage of the anti-PVRIG antibody, the anti-TIGIT antibody, and pembrolizumab exhibits a decreased and/or lower baseline serum level of IL-8 of no more than about 15 pg/mL.

In some embodiments, the subject treated with the therapeutically effective dosage of the anti-PVRIG antibody, the anti-TIGIT antibody, and pembrolizumab exhibits a decreased and/or lower baseline serum level of IL-8 for at least about 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, or 40 weeks. In some embodiments, the subject treated with the therapeutically effective dosage of the anti-PVRIG antibody, the anti-TIGIT antibody, and pembrolizumab exhibits a decreased and/or lower baseline serum level of IL-8 for at least about 24 weeks.

In some embodiments, the serum level of IL-8 can be employed as a biomarker or companion diagnostic for use in determining treatment regimens as well as predicting or determining treatment efficacy. In some embodiments, the serum level of IL-8 can be employed as a biomarker or companion diagnostic for use in the determining treatment regimens as well as predicting or determining treatment efficacy for treatment with the anti-PVRIG antibody of the invention, the anti-TIGIT antibody of the invention, and pembrolizumab. In some embodiments, the serum level of IL-8 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the subject exhibits a decreased and/or lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the baseline serum level of IL-8 is no more than about 25 pg/mL, when employed as a biomarker or companion diagnostic for use in the determining treatment regimens as well as predicting or determining treatment efficacy for treatment with the anti-PVRIG antibody of the invention, the anti-TIGIT antibody of the invention, and pembrolizumab. In some embodiments, the serum level of IL-8 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the subject exhibits a decreased and/or lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the baseline serum level of IL-8 is no more than about 25 pg/mL, is predictive of treatment efficacy.

In some embodiments, there is an increased prediction of a response to the combination of the anti-PVRIG antibody of the invention, the anti-TIGIT antibody of the invention, and pembrolizumab according to at least some embodiments of the present invention when the decreased and/or lower baseline serum level IL-8 as compared to normal level (such as the serum IL-8 level of an untreated subject or non-responsive subject) is detected.

In some embodiments, the serum level of IL-8 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the subject exhibits a decreased and/or lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the baseline serum level of IL-8 is no more than about 25 pg/mL, is indicative of an increased prediction of a response to the combination of the anti-PVRIG antibody of the invention, the anti-TIGIT antibody of the invention, and pembrolizumab.

In some embodiments, there is an increased prediction of a response to the combination of the anti-PVRIG antibody of the invention, the anti-TIGIT antibody of the invention, and pembrolizumab when the subject exhibits a lower baseline serum level of IL-8 as compared to a preset reference value. In some embodiments, the lower baseline serum level of IL-8 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the subject exhibits a decreased and/or lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the baseline serum level of IL-8 is no more than about 25 pg/mL. In some embodiments, there is an increased prediction of a response to the combination of the anti-PVRIG antibody of the invention, the anti-TIGIT antibody of the invention, and pembrolizumab when the subject exhibits a lower baseline serum level of IL-8 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL. In some embodiments, there is an increased prediction of a response to the combination of the anti-PVRIG antibody of the invention, the anti-TIGIT antibody of the invention, and pembrolizumab when the subject exhibits a lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL. In some embodiments, the subject treated with the therapeutically effective dosage of the anti-PVRIG antibody, the anti-TIGIT antibody, and pembrolizumab exhibits a lower baseline serum level of IL-8 of no more than about 25 pg/mL.

In some embodiments, the invention provides a method for determining a cancer patient population for treatment with the anti-PVRIG antibody of the invention, the anti-TIGIT antibody of the invention, and pembrolizumab the method comprising:
(a) detecting the presence of IL-8 in the serum from the cancer patient,
(b) quantitating the measurement of the level of IL-8;
(c) treating the cancer patient with the anti-PVRIG antibody of the invention, the anti-TIGIT antibody of the invention, and pembrolizumab when IL-8 is present at a decreased and/or lower baseline serum level as compared to a control or a patient that is not treated with or non-responsive to the combination.

In some embodiments, the invention provides a method for determining a cancer patient population for treatment with the anti-PVRIG antibody of the invention, the anti-TIGIT antibody of the invention, and pembrolizumab the method comprising:
(a) detecting the presence of IL-8 in the serum from the cancer patient,
(b) quantitating the measurement of the level of IL-8;
(c) treating the cancer patient with the anti-PVRIG antibody of the invention, the anti-TIGIT antibody of the invention, and pembrolizumab when IL-8 is present at a lower baseline serum level. In some embodiments, the lower baseline serum level of IL-8 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL. In some embodiments the lower baseline serum level of IL-8 is no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL. In some embodiments, the lower baseline serum level of IL-8 is no more than about 25 pg/mL.

In some embodiments, cancer patients having a decreased and/or lower baseline serum level of IL-8 of at least about 10%, 20%, 30%, 40%, 50%, 60%, 70%, 80%, 90%, or 100% relative to an untreated subject, or a non-responsive subject has a higher probability to respond to the combination of the anti-PVRIG antibody of the invention, the anti-TIGIT antibody of the invention, and pembrolizumab. In some embodiments, the anti-PVRIG antibody comprises the antibody sequence in FIG. 3, including for example, CHA.7.518.1.H4(S241P)vhCDR1, CHA.7.518.1.H4(S241P)vhCDR2, CHA.7.518.1.H4(S241P)vhCDR3, CHA.7.518.1.H4(S241P)vlCDR1, CHA.7.518.1.H4(S241P)vlCDR2, and CHA.7.518.1.H4(S241P)vlCDR3.

In some embodiments, cancer patients having a decreased and/or lower baseline serum level of IL-8 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally a decreased and/or lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL a decreased and/or lower baseline serum level of IL-8 of no more than about 25 pg/mL, have a higher probability to respond to the combination of the anti-PVRIG antibody of the invention, the anti-TIGIT antibody of the invention, and pembrolizumab, relative to cancer patients with a higher serum level of IL-8. In some embodiments, the anti-PVRIG antibody comprises the antibody sequence in FIG. 3, including for example, CHA.7.518.1.H4(S241P)vhCDR1, CHA.7.518.1.H4(S241P)vhCDR2, CHA.7.518.1.H4(S241P)vhCDR3, CHA.7.518.1.H4(S241P)vlCDR1, CHA.7.518.1.H4(S241P)vlCDR2, and CHA.7.518.1.H4(S241P)vlCDR3.

In some embodiments, cancer patients having a lower baseline serum level of IL-8 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the subject exhibits a lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally wherein the subject exhibits a lower baseline serum level of IL-8 of no more than about 25 pg/mL, have a higher probability to respond to the combination of the anti-PVRIG antibody of the invention, the anti-TIGIT antibody of the invention, and pembrolizumab, relative to cancer patients with a higher serum level of IL-8. In some embodiments, the anti-PVRIG antibody comprises the antibody sequence in FIG. 3, including for example, CHA.7.518.1.H4(S241P)vhCDR1, CHA.7.518.1.H4(S241P)vhCDR2, CHA.7.518.1.H4(S241P)vhCDR3, CHA.7.518.1.H4(S241P)vlCDR1, CHA.7.518.1.H4(S241P)vlCDR2, and CHA.7.518.1.H4(S241P)vlCDR3.

In some embodiments, cancer patients having a decreased and/or lower baseline serum level of IL-8 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally a decreased and/or lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally a decreased and/or lower baseline serum level of IL-8 of no more than about 25 pg/mL have a higher probability to respond to the combination of the anti-PVRIG antibody of the invention, the anti-TIGIT antibody of the invention, and pembrolizumab, relative to cancer patients with a higher serum level of IL-8. In some embodiments, cancer patients having a decreased and/or lower baseline serum level of IL-8 of no more than about 30 pg/mL have a higher probability to respond to the combination of the anti-PVRIG antibody of the invention, the anti-TIGIT antibody of the invention, and pembrolizumab, relative to cancer patients with a higher serum level of IL-8. In some embodiments, cancer patients having a decreased and/or lower baseline serum level of IL-8 of no more than about 25 pg/mL have a higher probability to respond to the combination of the anti-PVRIG antibody of the invention, the anti-TIGIT antibody of the invention, and pembrolizumab, relative to cancer patients with a higher serum level of IL-8. In some embodiments, cancer patients having a decreased and/or lower baseline serum level of IL-8 of no more than about 20 pg/mL have a higher probability to respond to the combination of the anti-PVRIG antibody of the invention, the anti-TIGIT antibody of the invention, and pembrolizumab, relative to cancer patients with a higher serum level of IL-8. In some embodiments, cancer patients having a decreased and/or lower baseline serum level of IL-8 of no more than about 15 pg/mL have a higher probability to respond to the combination of the anti-PVRIG antibody of the invention, the anti-TIGIT antibody of the invention, and pembrolizumab, relative to cancer patients with a higher serum level of IL-8.

In some embodiments, cancer patients having a lower baseline serum level of IL-8 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally a lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally a lower baseline serum level of IL-8 of no more than about 25 pg/mL have a higher probability to respond to the combination of the anti-PVRIG antibody of the invention, the anti-TIGIT antibody of the invention, and pembrolizumab, relative to cancer patients with a higher serum level of IL-8. In some embodiments, cancer patients having a lower baseline serum level of IL-8 of no more than about 30 pg/mL have a higher probability to respond to the combination of the anti-PVRIG antibody of the invention, the anti-TIGIT antibody of the invention, and pembrolizumab, relative to cancer patients with a higher serum level of IL-8. In some embodiments, cancer patients having a lower baseline serum level of IL-8 of no more than about 25 pg/mL have a higher probability to respond to the combination of the anti-PVRIG antibody of the invention, the anti-TIGIT antibody of the invention, and pembrolizumab, relative to cancer patients with a higher serum level of IL-8. In some embodiments, cancer patients having a lower baseline serum level of IL-8 of no more than about 20 pg/mL have a higher probability to respond to the combination of the anti-PVRIG antibody of the invention, the anti-TIGIT antibody of the invention, and pembrolizumab, relative to cancer patients with a higher serum level of IL-8. In some embodiments, cancer patients having a lower baseline serum level of IL-8 of no more than about 15 pg/mL have a higher probability to respond to the combination of the anti-PVRIG antibody of the invention, the anti-TIGIT antibody of the invention, and pembrolizumab, relative to cancer patients with a higher serum level of IL-8.

In some embodiments, cancer patients, the invention provides a method for predicting or determining the efficacy of treatment with the anti-PVRIG antibody of the invention, the anti-TIGIT antibody of the invention, and pembrolizumab, the method comprising:
(a) measuring the serum level of IL-8 in the cancer patient;
(b) quantitating the measurement of the serum level of IL-8;
(c) correlating the serum level of IL-8 with the efficacy of treatment, wherein IL-8 at a decreased and/or lower baseline serum level as compared to a control or a patient that is not treated with or non-responsive to the combination of the anti-PVRIG antibody of the invention, the anti-TIGIT antibody of the invention, and pembrolizumab, is indicative of treatment efficacy.

In some embodiments, the method further comprises (d) optionally treating the cancer patient with the anti-PVRIG antibody of the invention, the anti-TIGIT antibody of the invention, and pembrolizumab when IL-8 in (a) is present at a decreased and/or lower baseline serum level as compared to a control or a patient that is not treated with or non-responsive to the treatment.

In some embodiments, cancer patients, the invention provides a method for predicting or determining the efficacy of treatment with the anti-PVRIG antibody of the invention, the anti-TIGIT antibody of the invention, and pembrolizumab, the method comprising:
(a) measuring the serum level of IL-8 in the cancer patient;
(b) quantitating the measurement of the serum level of IL-8;
(c) correlating the serum level of IL-8 with the efficacy of treatment, wherein IL-8 at a lower baseline serum level as is indicative of treatment efficacy.

In some embodiments, the method further comprises (d) optionally treating the cancer patient with the anti-PVRIG antibody of the invention, the anti-TIGIT antibody of the invention, and pembrolizumab when IL-8 in (a) is present at a decreased and/or lower baseline serum level as compared to a control or a patient that is not treated with or non-responsive to the treatment. In some embodiments, the lower baseline serum level of IL-8 is no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally the lower baseline serum level of IL-8 is no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally the lower baseline serum level of IL-8 is no more than about 25 pg/mL.

In some embodiments, a decreased and/or lower baseline serum level of IL-8 of at least about 10%, 20%, 30%, 40%, 50%, 60%, 70%, 80%, 90%, or 100% relative to an untreated subject, or a non-responsive subject is indicative of efficacy of treatment with the anti-PVRIG antibody of the invention, the anti-TIGIT antibody of the invention, and pembrolizumab.

In some embodiments, a decreased and/or lower baseline serum level of IL-8 of no more than about 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally a decreased and/or lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally a decreased and/or lower baseline serum level of IL-8 of no more than about 25 pg/mL, is indicative of efficacy of treatment with the anti-PVRIG antibody of the invention, the anti-TIGIT antibody of the invention, and pembrolizumab.

In some embodiments, a decreased and/or lower baseline serum level of IL-8 of no more than about 30 pg/mL is indicative of efficacy of treatment with the anti-PVRIG antibody of the invention, the anti-TIGIT antibody of the invention, and pembrolizumab 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally a decreased and/or lower baseline serum level of IL-8 of no more than about 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 51 pg/mL, 52 pg/mL, 53 pg/mL, 54 pg/mL, 55 pg/mL, 56 pg/mL, 57 pg/mL, 58 pg/mL, 59 pg/mL, 60 pg/mL, 61 pg/mL, 62 pg/mL, 63 pg/mL, 64 pg/mL, 65 pg/mL, 66 pg/mL, 67 pg/mL, 68 pg/mL, 69 pg/mL, 70 pg/mL, 71 pg/mL, 72 pg/mL, 73 pg/mL, 74 pg/mL, 75 pg/mL, 76 pg/mL, 77 pg/mL, 78 pg/mL, 79 pg/mL, 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 91 pg/mL, 92 pg/mL, 93 pg/mL, 94 pg/mL, 95 pg/mL, 96 pg/mL, 97 pg/mL, 98 pg/mL, 99 pg/mL, or 100 pg/mL, optionally a decreased and/or lower baseline serum level of IL-8 of no more than about 25 pg/mL, is indicative of efficacy of treatment with the anti-PVRIG antibody of the invention, the anti-TIGIT antibody of the invention, and pembrolizumab. In some embodiments, a decreased and/or lower baseline serum level of IL-8 of no more than about 30 pg/mL is indicative of efficacy of treatment with the anti-PVRIG antibody of the invention, the anti-TIGIT antibody of the invention, and pembrolizumab. In some embodiments, a decreased and/or lower baseline serum level of IL-8 of no more than about 25 pg/mL is indicative of efficacy of treatment with the anti-PVRIG antibody of the invention, the anti-TIGIT antibody of the invention, and pembrolizumab. In some embodiments, a decreased and/or lower baseline serum level of IL-8 of no more than about 20 pg/mL is indicative of efficacy of treatment with the anti-PVRIG antibody of the invention, the anti-TIGIT antibody of the invention, and pembrolizumab. In some embodiments, a decreased and/or lower baseline serum level of IL-8 of no more than about 15 pg/mL is indicative of efficacy of treatment with the anti-PVRIG antibody of the invention, the anti-TIGIT antibody of the invention, and pembrolizumab. One of ordinary skill in the art would be able to determine a therapeutically effective amount based on such factors as the subject's size, the severity of the subject's symptoms, and the particular composition or route of administration selected.

### 4. Cancers for Treatment

The triple combinations comprising pembrolizumab, a TIGIT antibody, and a PVRIG antibody formulation of the invention find particular use in the treatment of cancer. In general, the antibodies of the invention are immunomodulatory, in that rather than directly attack cancerous cells, the antibodies of the invention stimulate the immune system. Thus, unlike tumor-targeted therapies, which are aimed at inhibiting molecular pathways that are crucial for tumor growth and development, and/or depleting tumor cells, cancer immunotherapy is aimed to stimulate the patient's or subject's own immune system to eliminate cancer cells, providing long-lived tumor destruction. Various approaches can be used in cancer immunotherapy, among them are therapeutic cancer vaccines to induce tumor-specific T cell responses, and immunostimulatory antibodies (i.e., antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients or subjects respond (as opposed to conventional or targeted therapy, where a large number of patients or subjects respond, but responses are transient).

By the time a tumor is detected clinically, it has already evaded the immune-defense system by acquiring immuneresistant and immunosuppressive properties and creating an immunosuppressive tumor microenvironment through various mechanisms and a variety of immune cells.

“Cancer,” as used herein, refers broadly to any neoplastic disease (whether invasive or metastatic) characterized by abnormal and uncontrolled cell division causing malignant growth or tumor (e.g., unregulated cell growth). The term “cancer” or “cancerous” as used herein should be understood to encompass any neoplastic disease (whether invasive, non-invasive or metastatic) which is characterized by abnormal and uncontrolled cell division causing malignant growth or tumor, non-limiting examples of which are described herein. This includes any physiological condition in mammals that is typically characterized by unregulated cell growth.

In some embodiments, triple combinations comprising pembrolizumab, a TIGIT antibody, and a PVRIG antibody formulation of the present invention can be used in the treatment of solid tumors (including, for example, cancers of the lung, liver, breast, brain, GI tract) and blood cancers (including for example, leukemia and preleukemic disorders, lymphoma, plasma cell disorders, including neoplasm) carcinoma, lymphoma, blastoma, sarcoma, and leukemia or lymphoid malignancies, including advanced cancer forms. In some embodiments, the cancer is early. In some embodiments, the cancer is advanced (including metastatic). In some embodiments, the cancers amenable for treatment of the invention include cancers that express or do not express PVRIG and further include non-metastatic or non-invasive, as well as invasive or metastatic cancers, including cancers where PVRIG expression by immune, stromal, or diseased cells suppresses antitumor responses and anti-invasive immune responses. In some embodiments, the anti-PVRIG formulations can be used for the treatment of vascularized tumors. In some embodiments, the cancer for treatment using the anti-PVRIG formulations of the present invention includes carcinoma, lymphoma, sarcoma, and/or leukemia. In some embodiments, the cancer for treatment using the anti-PVRIG formulations of the present invention includes vascularized tumors, melanoma, non-melanoma skin cancer (squamous and basal cell carcinoma), mesothelioma, squamous cell cancer, lung cancer, small-cell lung cancer, non-small cell lung cancer, neuroendocrine lung cancer (including pleural mesothelioma, neuroendocrine lung carcinoma), NSCL (large cell), NSCLC large cell adenocarcinoma, non-small cell lung carcinoma (NSCLC), NSCLC squamous cell, soft-tissue sarcoma, Kaposi's sarcoma, adenocarcinoma of the lung, squamous carcinoma of the lung, NSCLC with PDL1 >=50% TPS, neuroendocrine lung carcinoma, atypical carcinoid lung cancer, cancer of the peritoneum, esophageal cancer, hepatocellular cancer, liver cancer (including HCC), gastric cancer, stomach cancer (including gastrointestinal cancer), pancreatic cancer, glioblastoma, cervical cancer, ovarian cancer, urothelial cancer, bladder cancer, hepatoma, glioma, brain cancer (as well as edema, such as that associated with brain tumors), breast cancer (including, for example, triple negative breast cancer), testis cancer, testicular germ cell tumors, colon cancer, colorectal cancer (CRC), colorectal cancer MSS (MSS-CRC; microsatellite stable colorectal carcinoma/carcinoma), metastatic MSS-CRC, refractory metastatic MSS-CRC, MSS-CRC with liver metastasis, refractory MSS colorectal; MSS (microsatellite stable status), primary peritoneal cancer, microsatellite stable primary peritoneal cancer, platinum resistant microsatellite stable primary peritoneal cancer, CRC (MSS unknown), rectal cancer, endometrial cancer (including endometrial carcinoma), uterine carcinoma, salivary gland carcinoma, kidney cancer, renal cell cancer (RCC), renal cell carcinoma (RCC), gastro-esophageal junction cancer, prostate cancer, vulval cancer, thyroid cancer, hepatic carcinoma, carcinoid carcinoma, head and neck cancer, B-cell lymphoma (including non-Hodgkin's lymphoma, as well as low grade/follicular non-Hodgkin's lymphoma (NHL), small lymphocytic (SL) NHL, intermediate grade/follicular NHL, intermediate grade diffuse NHL, Diffuse Large B cell lymphoma, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia, Hodgkin's lymphoma (HD), chronic lymphocytic leukemia (CLL), acute lymphoblastic leukemia (ALL), T cell Acute Lymphoblastic Leukemia (T-ALL), Acute myeloid leukemia (AML), Hairy cell leukemia, chronic myeloblastic leukemia, multiple myeloma, post-transplant lymphoproliferative disorder (PTLD), abnormal vascular proliferation associated with phakomatoses, Meigs' syndrome, Merkel Cell cancer, MSI-high cancer, KRAS mutant tumors, adult T-cell leukemia/lymphoma, adenoid cystic cancer (including adenoid cystic carcinoma), malignant melanoma, pancreatic cancer, pancreatic adenocarcinoma, ovarian cancer (including ovarian carcinoma), primary peritoneal ovarian carcinoma, advanced epithelial ovarian cancer, advanced epithelial ovarian carcinoma, platinum resistant ovarian cancer, platinum sensitive epithelial ovarian cancer, platinum sensitive high grade ovarian cancer, platinum sensitive fallopian tube cancer, platinum sensitive peritoneal cancer, platinum sensitive primary peritoneal cancer, platinum sensitive ovarian cancer, high grade serous adenocarcinoma, pleural mesothelioma, cervical squamous cell carcinoma (cervical SCC), anal squamous cell carcinoma (anal SCC), carcinoma of unknown primary, gallbladder cancer, malignant melanoma, pleural mesothelioma, chordoma, endometrial sarcoma, chondrosarcoma, uterine sarcoma, uveal melanoma, amyloidosis, AL-amyloidosis, astrocytoma, and/or Myelodysplastic syndromes (MDS).

In some embodiments, the cancer for treatment using the triple combinations comprising pembrolizumab, a TIGIT antibody, and a PVRIG antibody formulation of the present invention includes a cancer selected from the group consisting of prostate cancer, liver cancer (HCC), rectal cancer, colorectal cancer (CRC), colorectal cancer MSS (MSS-CRC; microsatellite stable colorectal carcinoma/carcinoma), CRC (MSS unknown), metastatic MSS-CRC, refractory metastatic MSS-CRC, MSS-CRC with liver metastasis, ovarian cancer (including ovarian carcinoma), primary peritoneal ovarian carcinoma, advanced epithelial ovarian cancer, advanced epithelial ovarian carcinoma, platinum resistant ovarian cancer, platinum sensitive epithelial ovarian cancer, platinum sensitive high grade ovarian cancer, platinum sensitive fallopian tube cancer, platinum sensitive peritoneal cancer, platinum sensitive primary peritoneal cancer, platinum sensitive ovarian cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1 >=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

“Cancer therapy” herein refers to any method that prevents or treats cancer or ameliorates one or more of the symptoms of cancer. Typically such therapies will comprise administration of immunostimulatory anti-PVRIG antibodies (including antigen-binding fragments) either alone or in combination with chemotherapy or radiotherapy or other biologics and for enhancing the activity thereof, i.e., in individuals wherein expression of PVRIG suppresses antitumor responses and the efficacy of chemotherapy or radiotherapy or biologic efficacy. In some embodiments, the cancer therapy is a receptor tyrosine kinase inhibitor therapy. In some embodiments, the receptor tyrosine kinase inhibitor therapy comprises lenvatinib. In some embodiments, the subject administered the anti-PVRIG antibody and anti-TIGIT antibody in combination with pembrolizumab is refractory or resistant to treatment with at least two or more prior therapeutic treatments, wherein the prior therapeutic treatments include one or more of fluroropyrimidines (or fluoropyrimidines), irinotecan, oxaliplatin, mirvetuximab soravtansine-gynx, anti-PD-1, anti-PD-L1, anti-PD-L2, anti-CD96 antibody, anti-OX-40 antibody, anti-CD137 antibody, anti-LAG3, anti-TIM3, and/or anti-CTLA4 antibody therapies. In some embodiments, the subject administered the anti-PVRIG antibody and anti-TIGIT antibody in combination with pembrolizumab is refractory or resistant to treatment with at least two or more prior therapeutic treatments, wherein the two or more prior therapeutic treatments include fluroropyrimidines (or fluoropyrimidines), irinotecan, oxaliplatin, and/or mirvetuximab soravtansine-gynx. In some embodiments, the subject administered the anti-PVRIG antibody and anti-TIGIT antibody in combination with pembrolizumab is refractory or resistant to treatment with no more than three prior therapeutic treatments. In some embodiments, the subject administered the anti-PVRIG antibody and anti-TIGIT antibody in combination with pembrolizumab is refractory or resistant to treatment with at least three or more prior therapeutic treatments. In some embodiments, the subject administered the anti-PVRIG antibody and anti-TIGIT antibody in combination with pembrolizumab is refractory or resistant to treatment with at least three or more prior therapeutic treatments, where in the three prior therapeutic treatments include fluroropyrimidines (or fluoropyrimidines), irinotecan, oxaliplatin and/or mirvetuximab soravtansine-gynx. In some embodiments, the subject administered the anti-PVRIG antibody and anti-TIGIT antibody in combination with pembrolizumab has not had prior treatment with an anti-PD-1, anti-PD-L1, or anti-PD-L2-directed therapy. In some embodiments, the subject administered the anti-PVRIG antibody and anti-TIGIT antibody in combination with pembrolizumab has not had prior treatment with an immune checkpoint inhibitor, including anti-PD-1, anti-PD-L1, anti-PD-L2, anti-CD96 antibody, anti-OX-40 antibody, anti-CD137 antibody, anti-LAG3, anti-TIM3, and/or anti-CTLA4 antibody therapies.

5. Selected Triple Combination Treatment with Formulation Embodiments

In some embodiments, the present invention provides for treatment of cancer in a subject in need thereof by administration of pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4(S241P) (SEQ ID NO:4), and
   ii) a light chain variable domain comprising the vlCDR1, vlCDR2, and vlCDR3 from the light chain of CHA.7.518.1.H4(S241P) (SEQ ID NO:9); or
   i) a heavy chain variable domain comprising the vhCDR1, vhCDR2, and vhCDR3 from the heavy chain of CHA.7.538.1.H4(S241P) (SEQ ID NO:14), and
   ii) a light chain variable domain comprising the vlCDR1, vlCDR2, and vlCDR3 from the light chain of CHA.7.538.1.H4(S241P) (SEQ ID NO:19);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain variable domain comprising the vhCDR1, vhCDR2, and vhCDR3 from the heavy chain of CPA.9.083.H4(S241P) (SEQ ID NO:24), and
   ii) a light chain variable domain comprising the vlCDR1, vlCDR2, and vlCDR3 from the light chain of CPA.9.083.H4(S241P) (SEQ ID NO:29); or
   i) a heavy chain variable domain comprising the vhCDR1, vhCDR2, and vhCDR3 from the heavy chain of CPA.9.086.H4(S241P) (SEQ ID NO:34), and
   ii) a light chain variable domain comprising the vlCDR1, vlCDR2, and vlCDR3 from the light chain of CPA.9.086.H4(S241P) (SEQ ID NO:39), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4(S241P) (SEQ ID NO:4), and
   ii) a light chain variable domain comprising the vlCDR1, vlCDR2, and vlCDR3 from the light chain of CHA.7.518.1.H4(S241P) (SEQ ID NO:9); or
   i) a heavy chain variable domain comprising the vhCDR1, vhCDR2, and vhCDR3 from the heavy chain of CHA.7.538.1.H4(S241P) (SEQ ID NO:14), and
   ii) a light chain variable domain comprising the vlCDR1, vlCDR2, and vlCDR3 from the light chain of CHA.7.538.1.H4(S241P) (SEQ ID NO:19);
(a) an anti-PVRIG antibody;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain variable domain comprising the vhCDR1, vhCDR2, and vhCDR3 from the heavy chain of CPA.9.083.H4(S241P) (SEQ ID NO:24), and
   ii) a light chain variable domain comprising the vlCDR1, vlCDR2, and vlCDR3 from the light chain of CPA.9.083.H4(S241P) (SEQ ID NO:29); or
   i) a heavy chain variable domain comprising the vhCDR1, vhCDR2, and vhCDR3 from the heavy chain of CPA.9.086.H4(S241P) (SEQ ID NO:34), and
   ii) a light chain variable domain comprising the vlCDR1, vlCDR2, and vlCDR3 from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain is from the heavy chain of CHA.7.518.1.H4(S241P) (SEQ ID NO:4), and
   ii) a light chain variable domain is from the light chain of CHA.7.518.1.H4(S241P) (SEQ ID NO:9); or
   i) a heavy chain variable domain is from the heavy chain of CHA.7.538.1.H4(S241P) (SEQ ID NO:14), and
   ii) a light chain variable domain is from the light chain of CHA.7.538.1.H4(S241P) (SEQ ID NO:19);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain variable is from the heavy chain of CPA.9.083.H4(S241P) (SEQ ID NO:24), and
   ii) a light chain variable domain is from the light chain of CPA.9.083.H4(S241P) (SEQ ID NO:29); or
   i) a heavy chain variable domain is from the heavy chain of CPA.9.086.H4(S241P) (SEQ ID NO:34), and
   ii) a light chain variable domain is from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain is from the heavy chain of CHA.7.518.1.H4(S241P) (SEQ ID NO:4), and
   ii) a light chain variable domain is from the light chain of CHA.7.518.1.H4(S241P) (SEQ ID NO:9); or
   i) a heavy chain variable domain is from the heavy chain of CHA.7.538.1.H4(S241P) (SEQ ID NO:14), and
   ii) a light chain variable domain is from the light chain of CHA.7.538.1.H4(S241P) (SEQ ID NO:19);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain variable is from the heavy chain of CPA.9.083.H4(S241P) (SEQ ID NO:24), and
   ii) a light chain variable domain is from the light chain of CPA.9.083.H4(S241P) (SEQ ID NO:29); or
   i) a heavy chain variable domain is from the heavy chain of CPA.9.086.H4(S241P) (SEQ ID NO:34), and
   ii) a light chain variable domain is from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
   ii) a light chain comprising:
      a) a VL-CL, wherein the VL from CHA.7.518.1.H4(S241P) (SEQ ID NO:9) and wherein the CL region is from human kappa 2 light chain; or
   i) a heavy chain comprising:
      a) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   ii) a light chain comprising:
      a) a VL-CL, wherein the VL from CHA.7.538.1.H4(S241P) (SEQ ID NO:19) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain comprising:
      a) a VH-CH1-hinge-CH2-CH3, wherein the VH is from CPA.9.083.H4(S241P) (SEQ ID NO:24) and wherein the CH1-hinge-CH2-CH3 region is from IgG4; and
   ii) a light chain comprising:
      a) a VL-CL, wherein the VL from CPA.9.083.H4(S241P) (SEQ ID NO:29) and wherein the CL region is from human kappa 2 light chain; or
   i) a heavy chain comprising:
      a) a VH-CH1-hinge-CH2-CH3, wherein the VH is from CPA.9.086.H4(S241P) (SEQ ID NO:34) and wherein the CH1-hinge-CH2-CH3 region is from IgG4; and
   ii) a light chain comprising:
   a) a VL-CL, wherein the VL from CPA.9.086.H4(S241P) (SEQ ID NO:39) and wherein the CL region is from human kappa 2 light chain), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
   ii) a light chain comprising:
      a) a VL-CL, wherein the VL from CHA.7.518.1.H4(S241P) (SEQ ID NO:9) and wherein the CL region is from human kappa 2 light chain; or
   i) a heavy chain comprising:
      a) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   ii) a light chain comprising:
      a) a VL-CL, wherein the VL from CHA.7.538.1.H4(S241P) (SEQ ID NO:19) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain comprising:
      a) a VH-CH1-hinge-CH2-CH3, wherein the VH is from CPA.9.083.H4(S241P) (SEQ ID NO:24) and wherein the CH1-hinge-CH2-CH3 region is from IgG4; and
   ii) a light chain comprising:
      a) a VL-CL, wherein the VL from CPA.9.083.H4(S241P) (SEQ ID NO:29) and wherein the CL region is from human kappa 2 light chain; or
   i) a heavy chain comprising:
      a) a VH-CH1-hinge-CH2-CH3, wherein the VH is from CPA.9.086.H4(S241P) (SEQ ID NO:34) and wherein the CH1-hinge-CH2-CH3 region is from IgG4; and
   ii) a light chain comprising:
   a) a VL-CL, wherein the VL from CPA.9.086.H4(S241P) (SEQ ID NO:39) and wherein the CL region is from human kappa 2 light chain), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13); or
   i) a heavy chain comprising the heavy chain from CHA.7.538.1.H4(S241P) (SEQ ID NO:18); and
   ii) a light chain comprising the light chain from CHA.7.538.1.H4(S241P) (SEQ ID NO:23);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain comprising the heavy chain from CPA.9.083.H4(S241P) (SEQ ID NO:28); and
   ii) a light chain comprising the light chain from CPA.9.083.H4(S241P) (SEQ ID NO:33); or
   i) a heavy chain comprising the heavy chain from CPA.9.086.H4(S241P) (SEQ ID NO:38); and
   ii) a light chain comprising the light chain from CPA.9.086.H4(S241P) (SEQ ID NO:43)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13); or
   i) a heavy chain comprising the heavy chain from CHA.7.538.1.H4(S241P) (SEQ ID NO:18); and
   ii) a light chain comprising the light chain from CHA.7.538.1.H4(S241P) (SEQ ID NO:23);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain comprising the heavy chain from CPA.9.083.H4(S241P) (SEQ ID NO:28); and
   ii) a light chain comprising the light chain from CPA.9.083.H4(S241P) (SEQ ID NO:33); or
   i) a heavy chain comprising the heavy chain from CPA.9.086.H4(S241P) (SEQ ID NO:38); and
   ii) a light chain comprising the light chain from CPA.9.086.H4(S241P) (SEQ ID NO:43)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4(S241P) (SEQ ID NO:4), and
   ii) a light chain variable domain comprising the vlCDR1, vlCDR2, and vlCDR3 from the light chain of CHA.7.518.1.H4(S241P) (SEQ ID NO:9); or
   i) a heavy chain variable domain comprising the vhCDR1, vhCDR2, and vhCDR3 from the heavy chain of CHA.7.538.1.H4(S241P) (SEQ ID NO:14), and
   ii) a light chain variable domain comprising the vlCDR1, vlCDR2, and vlCDR3 from the light chain of CHA.7.538.1.H4(S241P) (SEQ ID NO:19);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain variable domain comprising the vhCDR1, vhCDR2, and vhCDR3 from the heavy chain of CPA.9.083.H4(S241P) (SEQ ID NO:24), and
   ii) a light chain variable domain comprising the vlCDR1, vlCDR2, and vlCDR3 from the light chain of CPA.9.083.H4(S241P) (SEQ ID NO:29); or
   i) a heavy chain variable domain comprising the vhCDR1, vhCDR2, and vhCDR3 from the heavy chain of CPA.9.086.H4(S241P) (SEQ ID NO:34), and
   ii) a light chain variable domain comprising the vlCDR1, vlCDR2, and vlCDR3 from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4(S241P) (SEQ ID NO:4), and
   ii) a light chain variable domain comprising the vlCDR1, vlCDR2, and vlCDR3 from the light chain of CHA.7.518.1.H4(S241P) (SEQ ID NO:9);
   i) a heavy chain variable domain comprising the vhCDR1, vhCDR2, and vhCDR3 from the heavy chain of CHA.7.538.1.H4(S241P) (SEQ ID NO:14), and
   ii) a light chain variable domain comprising the vlCDR1, vlCDR2, and vlCDR3 from the light chain of CHA.7.538.1.H4(S241P) (SEQ ID NO:19).
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain variable domain comprising the vhCDR1, vhCDR2, and vhCDR3 from the heavy chain of CPA.9.083.H4(S241P) (SEQ ID NO:24), and
   ii) a light chain variable domain comprising the vlCDR1, vlCDR2, and vlCDR3 from the light chain of CPA.9.083.H4(S241P) (SEQ ID NO:29); or
   i) a heavy chain variable domain comprising the vhCDR1, vhCDR2, and vhCDR3 from the heavy chain of CPA.9.086.H4(S241P) (SEQ ID NO:34), and
   ii) a light chain variable domain comprising the vlCDR1, vlCDR2, and vlCDR3 from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain is from the heavy chain of CHA.7.518.1.H4(S241P) (SEQ ID NO:4), and
   ii) a light chain variable domain is from the light chain of CHA.7.518.1.H4(S241P) (SEQ ID NO:9); or
   i) a heavy chain variable domain is from the heavy chain of CHA.7.538.1.H4(S241P) (SEQ ID NO:14), and
   ii) a light chain variable domain is from the light chain of CHA.7.538.1.H4(S241P) (SEQ ID NO:19);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain variable domain is from the heavy chain of CPA.9.083.H4(S241P) (SEQ ID NO:24), and
   ii) a light chain variable domain is from the light chain of CPA.9.083.H4(S241P) (SEQ ID NO:29); or
   i) a heavy chain variable domain is from the heavy chain of CPA.9.086.H4(S241P) (SEQ ID NO:34), and
   ii) a light chain variable domain is from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain is from the heavy chain of CHA.7.518.1.H4(S241P) (SEQ ID NO:4), and
   ii) a light chain variable domain is from the light chain of CHA.7.518.1.H4(S241P) (SEQ ID NO:9); Or
   i) a heavy chain variable domain is from the heavy chain of CHA.7.538.1.H4(S241P) (SEQ ID NO:14), and
   ii) a light chain variable domain is from the light chain of CHA.7.538.1.H4(S241P) (SEQ ID NO:19);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain variable domain is from the heavy chain of CPA.9.083.H4(S241P) (SEQ ID NO:24), and
   ii) a light chain variable domain is from the light chain of CPA.9.083.H4(S241P) (SEQ ID NO:29); or
   i) a heavy chain variable domain is from the heavy chain of CPA.9.086.H4(S241P) (SEQ ID NO:34), and
   ii) a light chain variable domain is from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
   ii) a light chain comprising:
      a) a VL-CL, wherein the VL from CHA.7.518.1.H4(S241P) (SEQ ID NO:9) and wherein the CL region is from human kappa 2 light chain; or
   i) a heavy chain comprising:
      a) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   ii) a light chain comprising:
      a) a VL-CL, wherein the VL from CHA.7.538.1.H4(S241P) (SEQ ID NO:19) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   a heavy chain comprising:
      a) a VH-CH1-hinge-CH2-CH3, wherein the VH is from CPA.9.083.H4(S241P) (SEQ ID NO:24) and wherein the CH1-hinge-CH2-CH3 region is from IgG4; and
   ii) a light chain comprising:
      a) a VL-CL, wherein the VL from CPA.9.083.H4(S241P) (SEQ ID NO:29) and wherein the CL region is from human kappa 2 light chain; or
   i) a heavy chain comprising:
      a) a VH-CH1-hinge-CH2-CH3, wherein the VH is from CPA.9.086.H4(S241P) (SEQ ID NO:34) and wherein the CH1-hinge-CH2-CH3 region is from IgG4; and
   ii) a light chain comprising:
   a) a VL-CL, wherein the VL from CPA.9.086.H4(S241P) (SEQ ID NO:39) and wherein the CL region is from human kappa 2 light chain), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
   ii) a light chain comprising:
      a) a VL-CL, wherein the VL from CHA.7.518.1.H4(S241P) (SEQ ID NO:9) and wherein the CL region is from human kappa 2 light chain; or
   i) a heavy chain comprising:
      a) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   ii) a light chain comprising:
      a) a VL-CL, wherein the VL from CHA.7.538.1.H4(S241P) (SEQ ID NO:19) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   a heavy chain comprising:
      a) a VH-CH1-hinge-CH2-CH3, wherein the VH is from CPA.9.083.H4(S241P) (SEQ ID NO:24) and wherein the CH1-hinge-CH2-CH3 region is from IgG4; and
   ii) a light chain comprising:
      a) a VL-CL, wherein the VL from CPA.9.083.H4(S241P) (SEQ ID NO:29) and wherein the CL region is from human kappa 2 light chain; or
   i) a heavy chain comprising:
      a) a VH-CH1-hinge-CH2-CH3, wherein the VH is from CPA.9.086.H4(S241P) (SEQ ID NO:34) and wherein the CH1-hinge-CH2-CH3 region is from IgG4; and
   ii) a light chain comprising:
   a) a VL-CL, wherein the VL from CPA.9.086.H4(S241P) (SEQ ID NO:39) and wherein the CL region is from human kappa 2 light chain), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13); or
   i) a heavy chain comprising the heavy chain from CHA.7.538.1.H4(S241P) (SEQ ID NO:18); and
   ii) a light chain comprising the light chain from CHA.7.538.1.H4(S241P) (SEQ ID NO:23);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain comprising the heavy chain from CPA.9.083.H4(S241P) (SEQ ID NO:28); and
   ii) a light chain comprising the light chain from CPA.9.083.H4(S241P) (SEQ ID NO:33); or
   i) a heavy chain comprising the heavy chain from CPA.9.086.H4(S241P) (SEQ ID NO:38); and
   ii) a light chain comprising the light chain from CPA.9.086.H4(S241P) (SEQ ID NO:43)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13); or
   i) a heavy chain comprising the heavy chain from CHA.7.538.1.H4(S241P) (SEQ ID NO:18); and
   ii) a light chain comprising the light chain from CHA.7.538.1.H4(S241P) (SEQ ID NO:23);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain comprising the heavy chain from CPA.9.083.H4(S241P) (SEQ ID NO:28); and
   ii) a light chain comprising the light chain from CPA.9.083.H4(S241P) (SEQ ID NO:33); or
   i) a heavy chain comprising the heavy chain from CPA.9.086.H4(S241P) (SEQ ID NO:38); and
   ii) a light chain comprising the light chain from CPA.9.086.H4(S241P) (SEQ ID NO:43)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4(S241P) (SEQ ID NO:4), and
   ii) a light chain variable domain comprising the vlCDR1, vlCDR2, and vlCDR3 from the light chain of CHA.7.518.1.H4(S241P) (SEQ ID NO:9); or
   i) a heavy chain variable domain comprising the vhCDR1, vhCDR2, and vhCDR3 from the heavy chain of CHA.7.538.1.H4(S241P) (SEQ ID NO:14), and
   ii) a light chain variable domain comprising the vlCDR1, vlCDR2, and vlCDR3 from the light chain of CHA.7.538.1.H4(S241P) (SEQ ID NO:19);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain variable domain comprising the vhCDR1, vhCDR2, and vhCDR3 from the heavy chain of CPA.9.083.H4(S241P) (SEQ ID NO:24), and
   ii) a light chain variable domain comprising the vlCDR1, vlCDR2, and vlCDR3 from the light chain of CPA.9.083.H4(S241P) (SEQ ID NO:29); or i) a heavy chain variable domain comprising the vhCDR1, vhCDR2, and vhCDR3 from the heavy chain of CPA.9.086.H4(S241P) (SEQ ID NO:34), and
   ii) a light chain variable domain comprising the vlCDR1, vlCDR2, and vlCDR3 from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain comprising the vhCDR1, vhCDR2, and vhCDR3 from the heavy chain of CHA.7.518.1.H4(S241P) (SEQ ID NO:4), and
   ii) a light chain variable domain comprising the vlCDR1, vlCDR2, and vlCDR3 from the light chain of CHA.7.518.1.H4(S241P) (SEQ ID NO:9);
   i) a heavy chain variable domain comprising the vhCDR1, vhCDR2, and vhCDR3 from the heavy chain of CHA.7.538.1.H4(S241P) (SEQ ID NO:14), and
   ii) a light chain variable domain comprising the vlCDR1, vlCDR2, and vlCDR3 from the light chain of CHA.7.538.1.H4(S241P) (SEQ ID NO:19);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain variable domain comprising the vhCDR1, vhCDR2, and vhCDR3 from the heavy chain of CPA.9.083.H4(S241P) (SEQ ID NO:24), and
   ii) a light chain variable domain comprising the vlCDR1, vlCDR2, and vlCDR3 from the light chain of CPA.9.083.H4(S241P) (SEQ ID NO:29); or i) a heavy chain variable domain comprising the vhCDR1, vhCDR2, and vhCDR3 from the heavy chain of CPA.9.086.H4(S241P) (SEQ ID NO:34), and
   ii) a light chain variable domain comprising the vlCDR1, vlCDR2, and vlCDR3 from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain is from the heavy chain of CHA.7.518.1.H4(S241P) (SEQ ID NO:4), and
   ii) a light chain variable domain is from the light chain of CHA.7.518.1.H4(S241P) (SEQ ID NO:9); or
   i) a heavy chain variable domain is from the heavy chain of CHA.7.538.1.H4(S241P) (SEQ ID NO:14), and
   ii) a light chain variable domain is from the light chain of CHA.7.538.1.H4(S241P) (SEQ ID NO:19);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain variable domain is from the heavy chain of CPA.9.083.H4(S241P) (SEQ ID NO:24), and
   ii) a light chain variable domain is from the light chain of CPA.9.083.H4(S241P) (SEQ ID NO:29); or
   i) a heavy chain variable domain is from the heavy chain of CPA.9.086.H4(S241P) (SEQ ID NO:34), and
   ii) a light chain variable domain is from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain variable domain is from the heavy chain of CHA.7.518.1.H4(S241P) (SEQ ID NO:4), and
   ii) a light chain variable domain is from the light chain of CHA.7.518.1.H4(S241P) (SEQ ID NO:9); or
   i) a heavy chain variable domain is from the heavy chain of CHA.7.538.1.H4(S241P) (SEQ ID NO:14), and
   ii) a light chain variable domain is from the light chain of CHA.7.538.1.H4(S241P) (SEQ ID NO:19);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain variable domain is from the heavy chain of CPA.9.083.H4(S241P) (SEQ ID NO:24), and
   ii) a light chain variable domain is from the light chain of CPA.9.083.H4(S241P) (SEQ ID NO:29); or
   i) a heavy chain variable domain is from the heavy chain of CPA.9.086.H4(S241P) (SEQ ID NO:34), and
   ii) a light chain variable domain is from the light chain of CPA.9.086.H4(S241P) (SEQ ID NO:39)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
   ii) a light chain comprising:
      a) a VL-CL, wherein the VL from CHA.7.518.1.H4(S241P) (SEQ ID NO:9) and wherein the CL region is from human kappa 2 light chain; or
   i) a heavy chain comprising:
      a) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   ii) a light chain comprising:
      a) a VL-CL, wherein the VL from CHA.7.538.1.H4(S241P) (SEQ ID NO:19) and wherein the CL region is from human kappa 2 light chain;
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain comprising:
      a) a VH-CH1-hinge-CH2-CH3, wherein the VH is from CPA.9.083.H4(S241P) (SEQ ID NO:24) and wherein the CH1-hinge-CH2-CH3 region is from IgG4; and
   ii) a light chain comprising:
      a) a VL-CL, wherein the VL from CPA.9.083.H4(S241P) (SEQ ID NO:29) and wherein the CL region is from human kappa 2 light chain; or
   i) a heavy chain comprising:
      a) a VH-CH1-hinge-CH2-CH3, wherein the VH is from CPA.9.086.H4(S241P) (SEQ ID NO:34) and wherein the CH1-hinge-CH2-CH3 region is from IgG4; and
   ii) a light chain comprising:
   a) a VL-CL, wherein the VL from CPA.9.086.H4(S241P) (SEQ ID NO:39) and wherein the CL region is from human kappa 2 light chain), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising:
      a) a VH-CH1-hinge-CH2-CH3, wherein the VH is from CHA.7.518.1.H4(S241P) (SEQ ID NO:4) and wherein the CH1-hinge-CH2-CH3 region is from IgG4; and
   ii) a light chain comprising:
      a) a VL-CL, wherein the VL from CHA.7.518.1.H4(S241P) (SEQ ID NO:9) and wherein the CL region is from human kappa 2 light chain; or
   i) a heavy chain comprising:
      a) a VH-CH1-hinge-CH2-CH3, wherein the VH is from CHA.7.538.1.H4(S241P) (SEQ ID NO:14) and wherein the CH1-hinge-CH2-CH3 region is from IgG4; and
   ii) a light chain comprising:
      a) a VL-CL, wherein the VL from CHA.7.538.1.H4(S241P) (SEQ ID NO:19) and wherein the CL region is from human kappa 2 light chain;
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain comprising:
      a) a VH-CH1-hinge-CH2-CH3, wherein the VH is from CPA.9.083.H4(S241P) (SEQ ID NO:24) and wherein the CH1-hinge-CH2-CH3 region is from IgG4; and
   ii) a light chain comprising:
      a) a VL-CL, wherein the VL from CPA.9.083.H4(S241P) (SEQ ID NO:29) and wherein the CL region is from human kappa 2 light chain; or
   i) a heavy chain comprising:
      a) a VH-CH1-hinge-CH2-CH3, wherein the VH is from CPA.9.086.H4(S241P) (SEQ ID NO:34) and wherein the CH1-hinge-CH2-CH3 region is from IgG4; and
   ii) a light chain comprising:
      a) a VL-CL, wherein the VL from CPA.9.086.H4(S241P) (SEQ ID NO:39) and wherein the CL region is from human kappa 2 light chain), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13); or
   i) a heavy chain comprising the heavy chain from CHA.7.538.1.H4(S241P) (SEQ ID NO:18); and
   ii) a light chain comprising the light chain from CHA.7.538.1.H4(S241P) (SEQ ID NO:23);
(b) from 10 mM to 100 mM histidine;
(c) from 30 mM to 100 mM NaCl;
(d) from 20 mM to 150 mM L-Arginine; and
(e) from 0.005% to 0.1% w/v polysorbate 80,
wherein the composition has a pH from 5.5 to 7.0; and
wherein the anti-TIGIT antibody comprises:
   i) a heavy chain comprising the heavy chain from CPA.9.083.H4(S241P) (SEQ ID NO:28); and
   ii) a light chain comprising the light chain from CPA.9.083.H4(S241P) (SEQ ID NO:33); or
   i) a heavy chain comprising the heavy chain from CPA.9.086.H4(S241P) (SEQ ID NO:38); and
   ii) a light chain comprising the light chain from CPA.9.086.H4(S241P) (SEQ ID NO:43)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the present invention provides for treatment of cancer in a subject in need thereof by administration of 200 mg/kg pembrolizumab, an anti-TIGIT, and a stable liquid pharmaceutical formulation of an anti-PVRIG antibody, wherein the anti-PVRIG antibody is administered at a dosage of about 0.01 mg/kg, 0.03 mg/kg, 0.1 mg/kg, 0.3 mg/kg, 1 mg/kg, 3 mg/kg, 10 mg/kg, 15 mg/kg, or 20 mg/kg, and wherein the stable liquid formulation of the anti-PVRIG antibody comprises:
(a) an anti-PVRIG antibody comprising:
   i) a heavy chain comprising the heavy chain from CHA.7.518.1.H4(S241P) (SEQ ID NO:8); and
   ii) a light chain comprising the light chain from CHA.7.518.1.H4(S241P) (SEQ ID NO:13); or
   i) a heavy chain comprising the heavy chain from CHA.7.538.1.H4(S241P) (SEQ ID NO:18); and
   ii) a light chain comprising the light chain from CHA.7.538.1.H4(S241P) (SEQ ID NO:23);
(b) about 25 mM histidine;
(c) about 60 mM NaCl;
(d) about 100 mM L-Arginine; and
(e) about 0.01% % w/v polysorbate 80,
wherein the composition has a pH from 6.5+/−0.2; and
wherein the anti-TIGIT antibody comprises:
   i) a heavy chain comprising the heavy chain from CPA.9.083.H4(S241P) (SEQ ID NO:28); and
   ii) a light chain comprising the light chain from CPA.9.083.H4(S241P) (SEQ ID NO:33); or
   i) a heavy chain comprising the heavy chain from CPA.9.086.H4(S241P) (SEQ ID NO:38); and
   ii) a light chain comprising the light chain from CPA.9.086.H4(S241P) (SEQ ID NO:43)), and
wherein the anti-TIGIT antibody is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody.

In some embodiments, the pembrolizumab is administered as a dosage of about 200 mg/kg. In some embodiments, the pembrolizumab is administered as a dosage of about 200 mg/kg every 3 weeks.

In some embodiments, pembrolizumab is administered as a dosage of about 2 mg/kg to 10 mg/kg. In some embodiments, pembrolizumab is administered as a dosage of about 2 mg/kg. In some embodiments, pembrolizumab is administered as a dosage of about 2 mg/kg. In some embodiments, pembrolizumab is administered as a dosage of about 3 mg/kg. In some embodiments, pembrolizumab is administered as a dosage of about 4 mg/kg. In some embodiments, pembrolizumab is administered as a dosage of about 5 mg/kg. In some embodiments, pembrolizumab is administered as a dosage of about 6 mg/kg. In some embodiments, pembrolizumab is administered as a dosage of about 7 mg/kg. In some embodiments, pembrolizumab is administered as a dosage of about 8 mg/kg. In some embodiments, pembrolizumab is administered as a dosage of about 9 mg/kg. In some embodiments, pembrolizumab is administered as a dosage of about 10 mg/kg.

In some embodiments, pembrolizumab is administered as a dosage of about no more than 2 mg/kg. In some embodiments, pembrolizumab is administered as a dosage of about 1 mg/kg to 2 mg/kg. In some embodiments, pembrolizumab is administered as a dosage of about 0.1 mg/kg to 1 mg/kg. In some embodiments, pembrolizumab is administered as a dosage of about 0.01 mg/kg to 0.1 mg/kg.

In some embodiments, pembrolizumab is administered as a dosage of about at least 10 mg/kg. In some embodiments, pembrolizumab is administered as a dosage of about 10 mg/kg to 20 mg/kg. In some embodiments, pembrolizumab is administered as a dosage of about 20 mg/kg to 30 mg/kg. In some embodiments, pembrolizumab is administered as a dosage of about 30 mg/kg to 40 mg/kg. In some embodiments, pembrolizumab is administered as a dosage of about 40 mg/kg to 50 mg/kg.

In some embodiments, pembrolizumab is administered about every 1 week to every 6 weeks. In some embodiments, pembrolizumab is administered about every week. In some embodiments, pembrolizumab is administered about every 2 weeks. In some embodiments, pembrolizumab is administered about every 3 weeks. In some embodiments, pembrolizumab is administered about every 4 weeks. In some embodiments, pembrolizumab is administered about every 5 weeks. In some embodiments, pembrolizumab is administered about every 6 weeks.

In some embodiments, pembrolizumab is administered as a dosage of about 2 mg/kg every 3 weeks. In some embodiments, pembrolizumab is administered as a dosage of about 10 mg/kg every 3 weeks. In some embodiments, pembrolizumab is administered as a dosage of about 200 mg every 3 weeks. In some embodiments, pembrolizumab is administered as a dosage of about 400 mg every 6 weeks.

In some embodiments, the pembrolizumab is administered over about 10 minutes, over about 15 minutes, over about 20 minutes, over about 25 minutes, over about 30 minutes, over about 35 minutes, or over about 40 minutes. In some embodiments, the pembrolizumab is administered over about 30 minutes+/−10 minutes.

Further disclosures of pembrolizumab are provided in https://www.accessdata.fda.gov/spl/data/157262d6-15e0-4b0a-968f-b99bab4aef50/157262d6-15e0-4bOa-968f-b99bab4aef50.xml, which is incorporated by reference herein in its entirety.

In some embodiments, the anti-PVRIG antibody, anti-TIGIT antibody, and pembrolizumab are administered in any order. In some embodiments, the anti-PVRIG antibody, anti-TIGIT antibody, and pembrolizumab are administered simultaneously. In some embodiments, the anti-PVRIG antibody, anti-TIGIT antibody, and pembrolizumab are administered sequentially. In some embodiments, the order of administration is anti-PVRIG antibody, anti-TIGIT antibody, and pembrolizumab. In some embodiments, the order of administration is anti-TIGIT antibody, anti-PVRIG antibody, and pembrolizumab. In some embodiments, the order of administration is pembrolizumab, anti-PVRIG antibody, and anti-TIGIT antibody. In some embodiments, the order of administration is pembrolizumab, anti-TIGIT antibody, and anti-PVRIG antibody. In some embodiments, the order of administration is anti-PVRIG antibody, pembrolizumab, and anti-TIGIT antibody. In some embodiments, the order of administration is anti-TIGIT antibody, pembrolizumab, and anti-PVRIG antibody.

### EXAMPLES

### Example 1: PVRIG Antibody Formulation Testing

The anti-PVRIG antibody that was formulated in each buffer (A and B) was spiked with the corresponding excipients to create the 20 conditions listed in FIG. 6. Each formulation was referred to by the Formulation ID.

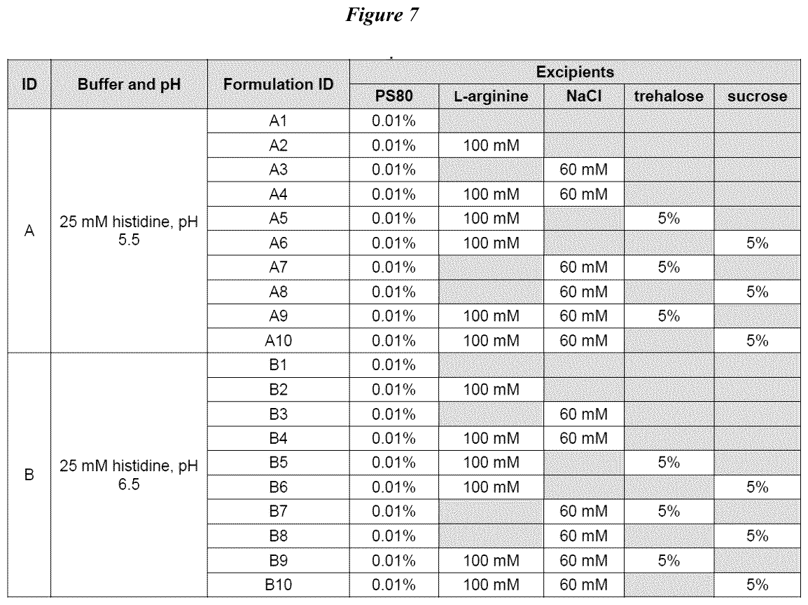

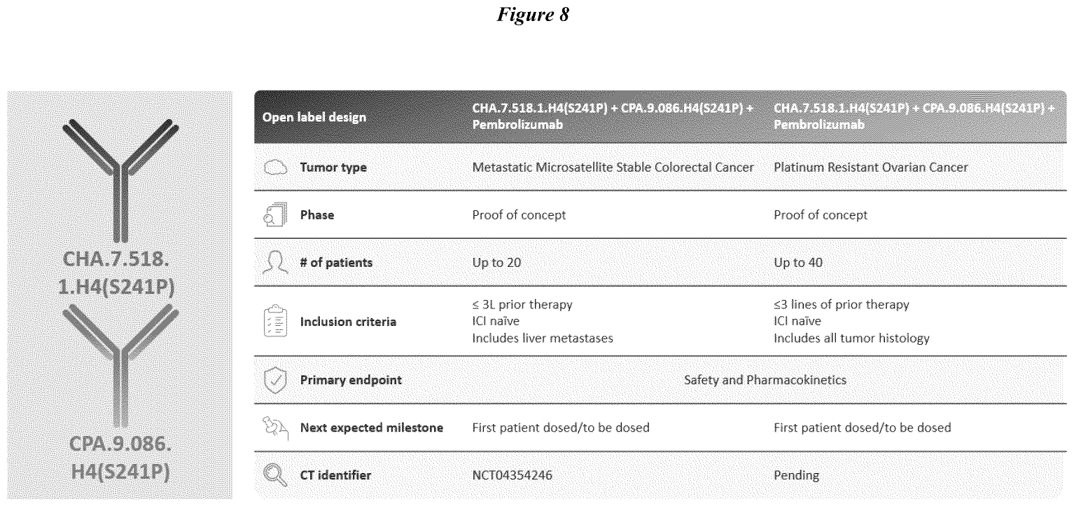

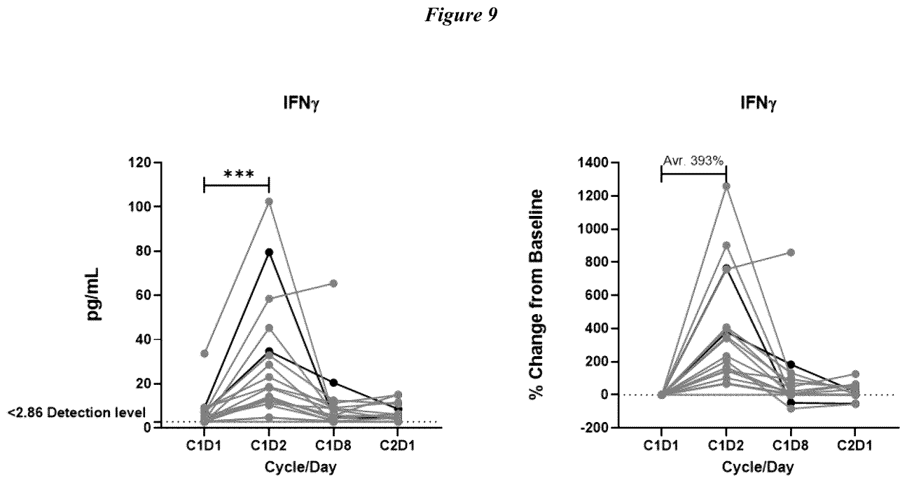

The formulated material was subjected to the stress and storage conditions in FIGS. 7A-7B.

### Formulation Study Procedural Steps

### Sampling Requirements

For each condition, a total of two vials were required for testing with one additional spare vial. One vial was required for LabChip (reduced and non-reduced), cIEF, concentration (A280 nm), the potency assay and SEC-HPLC analyses at CPS. The other vial was required for visual appearance and MFI analyses. Appropriate samples were removed at the indicated time points and frozen at <−60° C. until initiation of analysis, except samples for MFI and appearance testing. MFI and appearance testing which were performed immediately after pull.

The time zero (TO) samples of the 20 formulated samples were taken from the stock of vials stored at 2-8° C. and frozen at <−60° C. until analysis (or analyzed immediately in the case of appearance and MFI testing).

### Freeze/Thaw Study Storage at <−60° C.

For each cycle, three vials per formulation (total of 60 vials) were placed in storage at <−60° C. After a minimum of 16 hours, all three vials per formulation were pulled out of the <−60° C. storage condition (total of 60 vials) and allowed to warm to room temperature for 3-5 hours until thawed.

After being placed in the freezer for Cycle 3, the three vials were not thawed at room temperature until testing was ready to be initiated. Samples were then brought to room temperature prior to analysis.

Samples were assayed as described in this example.

### Agitation

Three vials from each of the 20 formulations (total of 60 vials) were placed and fixed on a shaker rotating at −200 rpm at room temperature. Vials were agitated for no less than 24 hours and no more than 48 hours. All vials were stored frozen at <−60° C. until analysis, except MFI and appearance testing, which was performed immediately. Samples were equilibrated to room temperature prior to analysis.

Samples were assayed as described in this example.

### 2-8° C. Storage for 8 Weeks (Testing at 0, 2, 4 and 8 Weeks)

Twelve vials were taken from each of the 20 formulations (a total of 240 vials including TO vials) and stored at 2-8° C. Two vials at TO were analyzed immediately for MFI and appearance. All other samples were labeled with temperature and time point and stored frozen at <−60° C. until analysis. Samples were brought to room temperature prior to analysis.

At each subsequent time point, three vials were taken per formulation out of the 2-8° C. storage condition, labeled with the temperature and time point and stored frozen at <−60° C. until analysis except MFI and appearance testing, which was performed immediately. Samples were brought to room temperature prior to analysis.

Samples were assayed as described in this example.

### Ambient Storage (25° C.) for 4 Weeks (Testing at 2 and 4 Weeks)

Six vials were taken from each of the 20 formulations (total 120 vials) and stored at ambient temperature (25° C.).

At each time point, three vials were taken per formulation out of ambient storage, labeled with temperature and time point and frozen at <−60° C. until analysis except MFI and appearance testing, which was performed immediately. Samples were brought to room temperature prior to analysis.

Samples were assayed as described in this example.

### 40° C. Storage for 2 Weeks (Testing at 1 and 2 Weeks)

Six vials were taken from each of the 20 formulations (total 120 vials) and stored at 40° C.

At each time point, three vials were taken per formulation out of 40° C. storage, labeled with temperature and time point and store frozen at <−60° C. until analysis except MFI appearance testing, which was performed immediately. Samples were brought to room temperature prior to analysis.

Samples were assayed as described in this example.

### Testing Plan and Schedule

For each formulation condition, sample and test were performed.

### Results

Each formulation was evaluated and compared to the conditions studied.

The critical assay results were compiled and analyzed. The critical assays analyzed to determine an appropriate formulation were SEC, cIEF and MFI. SEC high molecular and low molecular weight species were monitored throughout the study. cIEF results were obtained throughout the study. Finally, MFI particles/mL throughout the various sizes were monitored.

### Example 2: PVRIG Antibody Formulation

### Description and Composition of Solution for Infusion

The formulation is provided as a sterile preservative free liquid dosage form at a concentration of 20 mg/mL in a 10R Type I clear borosilicate glass vial equipped with a gray bromobutyl rubber stopper and aluminum flip cap crimp. The vials are filled to a target volume of 10 mL. The formulation is stored and shipped frozen at −20 C. Prior to use, the vials are thawed at ambient temperature, mixed by gentle swirling. For administration to patients, the formulation is diluted with 0.9% sodium chloride.

### Container Closure System

A single container closure system exists for the formulation and is comprised of a 10R Type I clear borosilicate glass vial, a 20 mm bromobutyl rubber stopper and a 20 mm aluminum flip cap crimp.

The formulation was produced by thawing and pooling the Drug Substance, followed by 0.22 μm sterile filtration and filling into sterile 10R glass vials at Vetter.

The formulation components and quantitative composition of the drug product on a nominal amount per vial unit (10 mL) is presented in the Table 1 below.

TABLE 1
Composition

| | | | Nominal |
|---|---|---|---|
| | | | amount |
| | | Presentation | [mg/vial] |
| | | 10R Glass | V = |
| Component | Function | Vial | 10.0 mL |
| CHA.7.518.1.H4(S241P) | Active | 20 mg/mL/mL | 200 |
| mAb | Ingredient | in liquid | |
| | | dosage form | |
| 25 mM | Buffer | 25 mM | 39 |
| Histidine | Component - | in liquid | |
| | pH | formulation | |
| | Stabilization | | |
| 60 mM | Buffer | 60 mM | 35 |
| NaCl | Component- | in liquid | |
| | Ionic Strength | formulation | |
| | Control | | |
| 100 mM L- | Formulation | 100 mM in | 174 |
| Arginine | Excipient- | liquid | |
| | Stabilizer | formulation | |
| Polysorbate 80 | Formulation | 0.01% w/v in | 1 |
| | Excipient | liquid | |
| | Surfactant | formulation | |

A sufficient volume is filled into vials based on the net fill weight to ensure a withdrawable volume of 10 mL.

### Example 3: A Phase 1 Study of the Safety and Tolerability of CPA.9.086.14(S241P) in Subjects with Advanced Malignancies

Phase 1 open label sequential dose escalation and cohort expansion study evaluating the safety, tolerability and preliminary antitumor activity of CPA.9.086.H4(S241P) as monotherapy and in combination with CHA.7.518.1.H4(S241P) in subjects with advanced malignancies.

Conditions for treatment: Advanced Cancer, Ovarian Cancer, Lung Cancer, Colon Cancer, Plasma Cell Neoplasm, Multiple Myeloma, HNSCC, Microsatellite Stable Colorectal Carcinoma, MSS-CRC, Metastatic MSS-CRC, or Refractory MSS-CRC or Platinum Resistant Ovarian Cancer.

Arm 1: Dose escalation: CPA.9.086.H4(S241P) monotherapy. CPA.9.086.H4(S241P) monotherapy administered IV every 3 weeks in sequential dose escalation doses in cohorts of subjects. Up to 7 dose escalation cohorts may be evaluated until a maximum tolerated dose or recommended dose for expansion (RDFE) is identified.

Arm 2: Combination Product: Evaluation of safety/tolerability: CPA.9.086.H4(S241P) in combination with CHA.7.518.1.H4(S241P) (both at the RDFE). Both study drugs will be evaluated at the RDFE for assessment of safety and tolerability. All study drugs will be administered IV every 3 weeks. Dual combination (CPA.9.086.H4(S241P)+CHA.7.518.1.H4(S241P)) for evaluation of safety/tolerability (both at RDFE). CPA.9.086.H4(S241P) will be combined with CHA.7.518.1.H4(S241P) for evaluation of safety and tolerability. All study drugs will be administered IV every 3 weeks.

Arm 3: CPA.9.086.H4(S241P) (RDFE) monotherapy. CPA.9.086.H4(S241P) monotherapy (RDFE) in subjects with multiple myeloma. CPA.9.086.H4(S241P) will be administered IV every 3 weeks. CPA.9.086.H4(S241P) monotherapy at the RDFE—in subjects with multiple myeloma. CPA.9.086.H4(S241P) will be administered IV every 3 weeks.

Arm 4: CPA.9.086.H4(S241P) in combination with CHA.7.518.1.H4(S241P) (both at the RDFE). CPA.9.086.H4(S241P) in combination with CHA.7.518.1.H4(S241P) (both at RDFE) in subjects with select tumor types who have exhausted standard treatment—HNSCC, CRC (MSS), NSCLC. All study drugs will be administered IV every 3 weeks. CPA.9.086.H4(S241P) in combination with CHA.7.518.1.H4(S241P) (both at the RDFE).

Arm 5: Combination Product: Triplet combination of CPA.9.086.H4(S241P)+CHA.7.518.1.H4(S241P)+Pembrolizumab in subjects with MSS-CRC, Metastatic MSS-CRC, or Refractory MSS-CRC or Platinum Resistant Ovarian Cancer. Triplet combination of CPA.9.086.H4(S241P)+CHA.7.518.1.H4(S241P)+Pembrolizumab evaluated in subjects with MSS-CRC. All study drugs will be administered IV every 3 weeks.

### Key Inclusion Criteria:

Subjects with histologically/cytologically confirmed advanced malignancy (solid tumor) who must have exhausted all available standard therapy, or not a candidate for standard therapy.

Subject is able to provide written, informed consent before initiation of any study related procedures, and is able, in the opinion of the investigator, to comply with all the requirements of the study.

Subject has Eastern Cooperative Oncology Group (ECOG) performance status 0-1.

### For Triplet Combination MSS-CRC:

Histologically confirmed adenocarcinoma of the colon/rectum

Stage IV disease

MSS-CRC status by an FDA approved test

Disease progression with no more than 3 prior lines of treatment including fluroropyrimidines (or fluoropyrimidines), irinotecan, and oxaliplatin or more than 3 prior lines of treatment including fluroropyrimidines (or fluoropyrimidines), irinotecan, and oxaliplatin.

### Key Exclusion Criteria:

Prior treatment with a TIGIT inhibitor.

Prior treatment with an inhibitor of PVRIG

Symptomatic interstitial lung disease or inflammatory pneumonitis.

History of immune-related events that required immunotherapy treatment discontinuation

For Triplet combination MSS-CRC cohort: No prior treatment with anti-PD-1, anti-PD-L1, or anti-PD-L2-directed therapy.

### Primary Outcome Measures

The safety and tolerability of CPA.9.086.H4(S241P) monotherapy and in combination with CHA.7.518.1.H4(S241P). [Time Frame: DLT evaluation window in the 1st cycle (21 Days).]

Incidence of subjects with Adverse Events (AEs) as per CTCAE v5.0 and Dose-Limiting Toxicities (DLTs).

To identify the maximum tolerated dose (MTD) and/or recommended dose for expansion of CPA.9.086.H4(S241P) monotherapy and in combination with CHA.7.518.1.H4(S241P). [Time Frame: 18 months.]

Evaluation of a dose of CPA.9.086.H4(S241P) monotherapy and in combination with CHA.7.518.1.H4(S241P) that is well tolerated by subjects.

To characterize the pharmacokinetic (PK) profile of CPA.9.086.H4(S241P) as monotherapy and in combination with CHA.7.518.1.H4(S241P). [Time Frame: 18 months.

Evaluation of parameters of CPA.9.086.H4(S241P) monotherapy or in combination with CHA.7.518.1.H4(S241P) exposure such as Maximum Plasma Concentration [Cmax]).

Evaluation of safety and tolerability of the Triplet combination (CPA.9.086.H4(S241P)+CHA.7.518.1.H4(S241P)+Pembrolizumab). [Time Frame: 18 months.]

Incidence of subjects on the Triplet combination (CPA.9.086.H4(S241P)+CHA.7.518.1.H4(S241P)+Pembrolizumab) with Adverse Events (AEs) per CTCAE v5.0.

Evaluation of the PK profile of the Triplet combination (CPA.9.086.H4(S241P)+CHA.7.518.1.H4(S241P)+Pembrolizumab). [Time Frame: 18 months.]

Evaluation of PK parameters e.g., Cmax.

Evaluation of the PK profile of the Triplet combination (CPA.9.086.H4(S241P)+CHA.7.518.1.H4(S241P)+Pembrolizumab). [Time Frame: 18 months.]

Evaluation of PK parameters, e.g., AUC.

### Secondary Outcome Measures

To characterize immunogenicity of CPA.9.086.H4(S241P) monotherapy and in combination with CHA.7.518.1.H4(S241P). [Time Frame: 18 months.]

Evaluation of anti-drug antibody to CPA.9.086.H4(S241P) (monotherapy) or CPA.9.086.H4(S241P), CHA.7.518.1.H4(S241P) when administered in combination.

To characterize the immunogenicity of the Triplet combination (CPA.9.086.H4(S241P)+CHA.7.518.1.H4(S241P)+Pembrolizumab). [Time Frame: 18 months.]

Evaluation of antidrug antibody to CPA.9.086.H4(S241P), CHA.7.518.1.H4(S241P).

### Original Secondary Outcome Measures ICMJE

To characterize immunogenicity of CPA.9.086.H4(S241P) monotherapy [Time Frame: 18 months.]

Evaluation of anti-drug antibody to CPA.9.086.H4(S241P).

Current Other Pre-specified Outcome Measures

Evaluation of the preliminary antitumor activity of CPA.9.086.H4(S241P) as monotherapy and in combination with CHA.7.518.1.H4(S241P). [Time Frame: 24 months.]

An assessment of preliminary antitumor activity eg ORR with CPA.9.086.H4(S241P) monotherapy and CPA.9.086.H4(S241P) in combination with CHA.7.518.1.H4(S241P).

Preliminary antitumor activity of the triplet combination (CPA.9.086.H4(S241P)+CHA.7.518.1.H4(S241P)+Pembrolizumab). [Time Frame: 24 months.]

Assessment of preliminary antitumor activity, e.g., ORR.

### Example 4: CRC (MSS) Triplet Expansion Cohort: Cpa.9.086.H4(S241P)+CHA.7.518.1.H4(S241P)+Pembrolizumab

### Exclusion Criterion

Prior treatment with an anti-PD-1/PD-L1/PD-L2, anti-CD96 antibody, anti-OX-40 antibody, anti-CD137 antibody, anti-LAG3, anti-TIM3, or anti-CTLA4 antibody.

### Example 5: Clinical Study of Ovarian Cancer (Triplet Expansion)

### Inclusion Criteria

Subjects with advanced epithelial ovarian cancer, fallopian tube cancer, or primary peritoneal carcinoma are eligible.

Platinum resistant ovarian cancer (PROC) defined as disease recurrence <6 months after completion of a platinum-containing regimen. Subjects with primary platinum refractory disease are ineligible. Primary platinum refractory disease is defined as progression of disease prior to completion of 1st line platinum therapy or immediately following (≤3 months following last date of chemotherapy).

Received ≤3 prior lines for PROC; maintenance bevacizumab or PARP are not included as a line of therapy.

Subjects who have received PARP inhibitor therapy are eligible.

### Exclusion Criteria

Subjects with prior treatment with an anti-PD-1/PD-L1/PD-L2, anti-CD96 antibody, anti-OX-40 antibody, anti-CD137 antibody, anti-LAG3, anti-TIM3, or anti-CTLA4 antibody are ineligible.

### EXAMPLE 6: CHA.7.518.1.H4(S241P) in Combination with CPA.9.086.H4(S241P) and Pembrolizumab Demonstrates Preliminary Antitumor Activity in Patients with MSS-CRC and Liver Metastases

Background: There is a high unmet need for the treatment of patients (pts) with metastatic microsatellite stable colorectal cancer (MSS-CRC). This example reports encouraging antitumor activity with the combination of CHA.7.518.1.H4(S241P)+nivolumab in patients with MSS-CRC and liver metastases [ 12/22(77%)], [ORR 2/17 (12%); disease control rate [DCR] 5/17 (29%)][1]. CHA.7.518.1.H4(S241P) is a novel, 1st-in-class immune checkpoint inhibitor (ICI) that binds to PVRIG, a DNAM-1 axis member, leading to activation of T and NK-cells; CPA.9.086.H4(S241P) is an ICI of TIGIT. Pembrolizumab is an ICI of PD-1. It is hypothesized that in patients with metastatic MSS-CRC, the triple combination by inhibiting the DNAM-1 axis would demonstrate antitumor activity with a favorable safety and tolerability profile.

Methods: 20 patients were enrolled with metastatic MSS-CRC who all received CHA.7.518.1.H4(S241P) 15 mg/kg+CPA.9.086.H4(S241P) 3 mg/kg+pembrolizumab 200 mg all IV Q3W. Primary objectives were safety/tolerability, with secondary objective of antitumor activity of the combination. Key inclusion criteria: Age ≥18 yrs, measurable disease, MSS by IHC or genomic testing, ≤3 prior lines including fluroropyrimidines, irinotecan, and oxaliplatin. Key exclusion criteria: prior receipt of ICI including anti-PVRIG, anti-TIGIT. Responses were investigated per RECIST v1.1, safety per CTCAE v5.0.

Results: Median age 57.5 yrs, 11/20 [F], 15/20 [75%] pts with liver metastases. Median of 3 prior lines of therapy. Objective response rate was [ORR] 1/20 [5%] patients; 7 patients with stable disease (SD). Disease control rate (DCR) [CR+PR+SD] 8/20 [40%] (CR=complete response; PR=partial response). In patients with liver metastases ORR 1/15 [7%], DCR 6/15 (40%); 6/8 [75%] pts with best response ≥SD had liver mets. Treatment related adverse events (TRAEs) were reported in 11/20 [55%] patients, the majority were ≤G2 7/20 [35%] with the most frequent TRAE of 4 patients each with ≤G2 fatigue, myalgia, 4/20 patients with G3 TRAE, there were no ≥G4 TRAEs. Strong peripheral IFNγ induction was observed after treatment. Three patients [1 PR, 2 SD] are ongoing at the time of data cut.

Conclusions: The data further supports the antitumor activity of the combination of CHA.7.518.1.H4(S241P)+CPA.9.086.H4(S241P)+pembrolizumab in patients with MSS-CRC and specifically in patients with liver metastases. Of note the data further reinforces the data previously disclosed in a similar patient population of 22 patients that received CHA.7.518.1.H4(S241P)+nivolumab [1]. No new safety signals were reported.

### CITED REFERENCES

1. Rasco D, Dumbrava E, Sharma M, et al 659 COM701 plus nivolumab demonstrates preliminary antitumor activity and immune modulation of tumor microenvironment in patients with metastatic MSS-CRC and liver metastases. Journal for ImmunoTherapy of Cancer 2022; 10:doi: 10.1136/jitc-2022-SITC2022.0659.

### EXAMPLE 7: CHA.7.518.1.H4(S241P) in Combination with CPA.9.086.H4(S241P) and Pembrolizumab Demonstrates Preliminary Antitumor Activity in Patients with MSS-CRC and Liver Metastases

### Introduction

There is a high unmet need for new treatments for patients with metastatic microsatellite stable colorectal cancer (MSS-CRC), which represents a challenging population to treat.
Current 3rd-line standard of care trifluridine/tipiracil (Lonsurf®)+bevacizumab demonstrated an objective response rate (ORR) of 6.1%, median progression-free survival (mPFS) of 5.6 months, and median overall survival (OS) of 10.8 months. See, Prager G W, et al. N Engl J Med. 2023; 388(18):1657-1667.
Most patients (˜70%) with advanced MSS-CRC have liver metastases and do not respond well to immune checkpoint inhibitor (ICI) therapy. See, Chen E X, et al. JAMA Netw Open. 2023; 6(12):e2346094.
MSS-CRC is considered to be a tumor of lower immunogenicity and the presence of liver metastases results in an additional mechanism of acquired ICI resistance. See, Huyghe N, et al. Cancers 2022; 14(9): 2241; and Yu J, et al. Nat Med. 2021; 27(1):152-164.

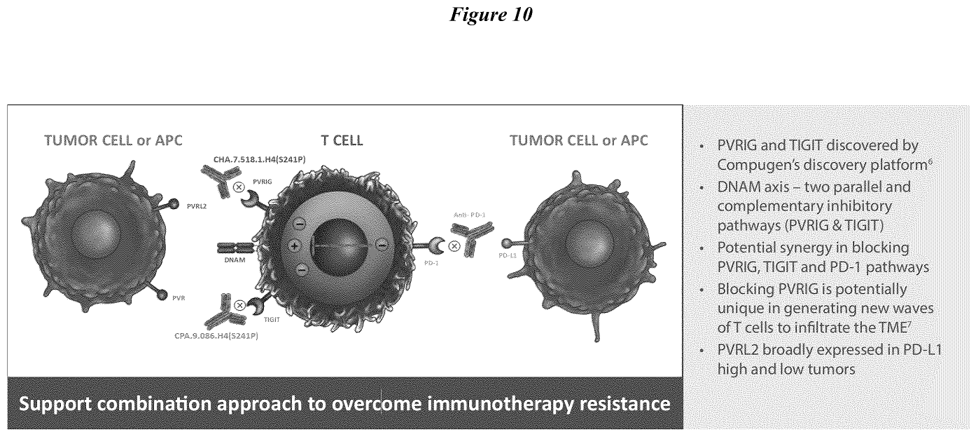

CHA.7.518.1.H4(S241P) is a novel, 1st-in-class ICI that inhibits PVRIG, a DNAM-1 axis member (FIG. 10), leading to activation of T and NK-cells. CPA.9.086.H4(S241P) is an ICI that inhibits the DNAM-1 family member TIGIT.

In a prior phase I clinical trial, the combination of CHA.7.518.1.H4(S241P)+nivolumab demonstrated encouraging preliminary antitumor activity in participants with MSS-CRC (N=22), 77% of whom had liver metastases. See, Rasco D, et al. J Immunother Cancer. 2022; 10(Suppl 2):A1-A1603.
Among participants with liver metastases, ORR was 12% (n= 2/17), 2 participants with PR, and disease control rate (DCR) was 24% ( 4/17). See, Rasco D, et al. J Immunother Cancer. 2022; 10(Suppl 2):A1-A1603.

It is hypothesized that a triplet combination of CHA.7.518.1.H4(S241P) and CPA.9.086.H4(S241P) plus pembrolizumab (anti-PD1) (see, Keytruda® (pembrolizumab) [Prescribing information]. Merck & Co., Inc., Rahway, NJ. 2024; and Schalper K A, et al. Nat Med. 2020; 26(5):688-692) would demonstrate antitumor activity by inhibiting the DNAM-1 axis in participants with metastatic MSS-CRC, and that this combination would have a favorable safety and tolerability profile.

### Objective

This example reports preliminary results on safety, tolerability, translational pharmacodynamics, and anti-tumor activity of the triplet combination (CHA.7.518.1.H4(S241P)+CPA.9.086.H4(S241P)+pembrolizumab) in patients with MSS-CRC, including patients with liver metastases.

### Methods

### Trial Design

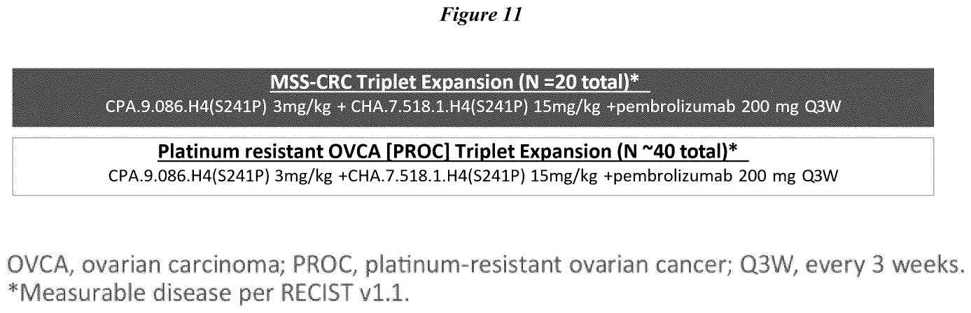

Data are from the MSS-CRC triplet expansion cohort of an ongoing phase I, open-label, multi-center, dose-escalation and expansion trial that is being conducted (FIG. 11).

Tumors are assessed (per RECIST v1.1) every 6 weeks for the first 6 cycles and every 12 weeks thereafter, or upon suspicion of progressive disease.

Trial treatment is administered for up to 35 cycles (˜24 months), until disease progression, unacceptable toxicity, initiation of new anticancer therapy, withdrawal of consent, or death.
To account for the phenomenon of pseudo-progression or delayed response with ICI, participants may continue to receive trial treatment beyond RECIST-defined progression at the discretion of the investigator and be assessed again within 6-8 weeks from initial progression to confirm disease progression.
Participants with confirmed disease progression per RECIST but with clinical benefit as per investigator assessment may continue to receive the investigational products.

Blood inflammatory markers (i.e., serum cytokines) including IFN-7, IL-6 and IL-8 levels were tested on C1D1 pre-infusion, C1D2, C1D8 and C2D1 pre-infusion.

Participants were required to have a tumor biopsy taken prior to first dose and after the second dose (but prior to third dose).

### Trial Objectives

Key primary objective: evaluate the safety and tolerability of the triplet combination (CHA.7.518.1.H4(S241P)+CPA.9.086.H4(S241P)+pembrolizumab), based on adverse event (AE) monitoring per NCI CTCAE v5.0.

Secondary objective: characterize immunogenicity of CPA.9.086.H4(S241P) in combination with CHA.7.518.1.H4(S241P) and pembrolizumab.

Prespecified exploratory objectives included:
Preliminary antitumor activity of the triplet combination (CHA.7.518.1.H4(S241P)+CPA.9.086.H4(S241P)+pembrolizumab).
Pharmacodynamic effects of trial drug(s) in the tumor and in blood, including changes in serum cytokine levels.

### Participants

### Key Inclusion Criteria:

Age≥18 years
Stage IV histologically confirmed adenocarcinoma of the colon/rectum
Measurable disease as defined by RECIST v1.1
Documented MSS status by an FDA approved test (e.g. genomic testing or immunohistochemistry for mismatch repair proficient)
Disease progression with ≤3 prior lines of treatment including fluroropyrimidines, irinotecan, and oxaliplatin
No prior treatment with an anti-PD-1/PD-L1/2 agent, anti-CD96 antibody, anti-OX-40 antibody, anti-CD137 antibody, anti-LAG3 agent, anti-TIM3 agent, or anti-CTLA4 antibody.

### Key Exclusion Criteria:

Prior receipt of ICI, including anti-PVRIG or anti-TIGIT therapy.
History of immune-related toxicities on prior immunotherapy treatment leading to discontinuation.

### Results

### Participant Baseline Characteristics and Disposition

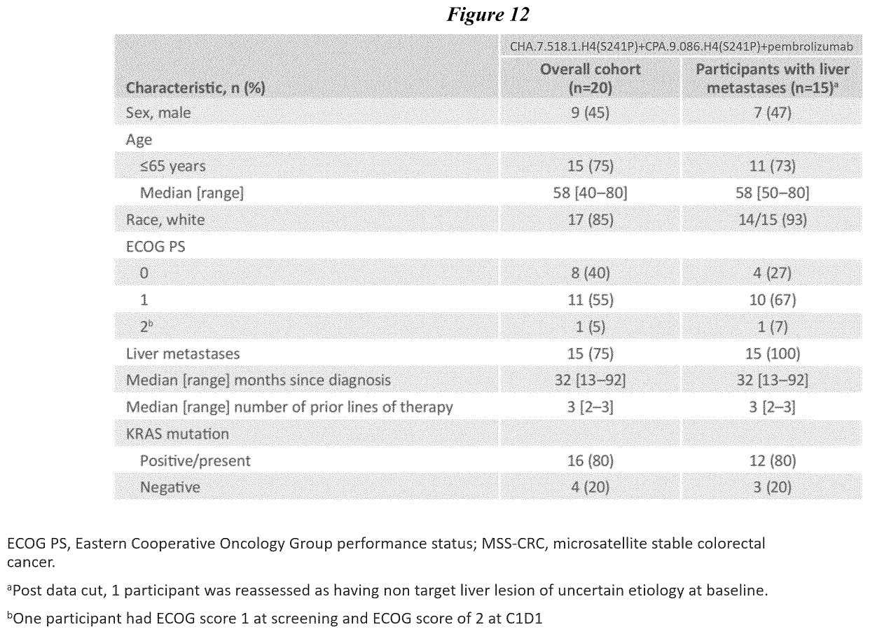

Baseline characteristics for the 20 participants with MSS-CRC are summarized in FIG. 12.

Most ( 15/20[75%]) participants had liver metastases at baseline:

Demographics were similar between the entire cohort and subset of participants with liver metastases.

All participants were previously treated with cytotoxic therapy, and none reported prior anti-PD-1/PDL1 therapy (as per eligibility criteria).

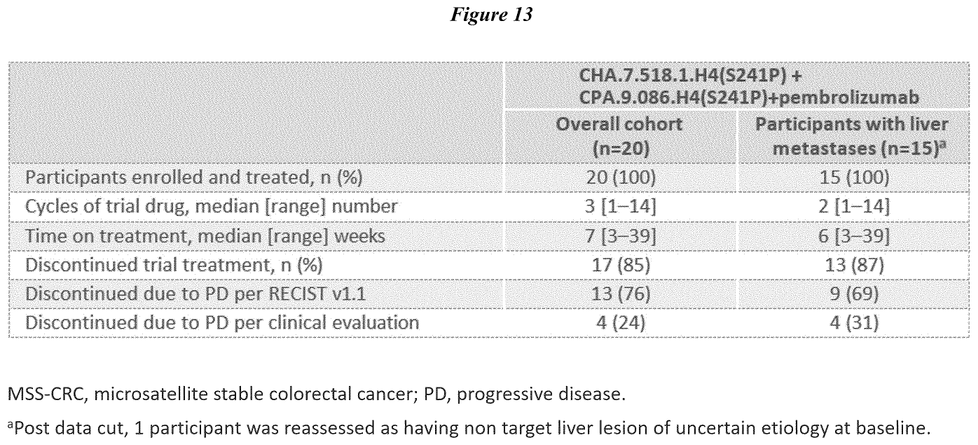

17 participants (85%) had discontinued trial treatment, all due to disease progression (FIG. 13).

### Safety

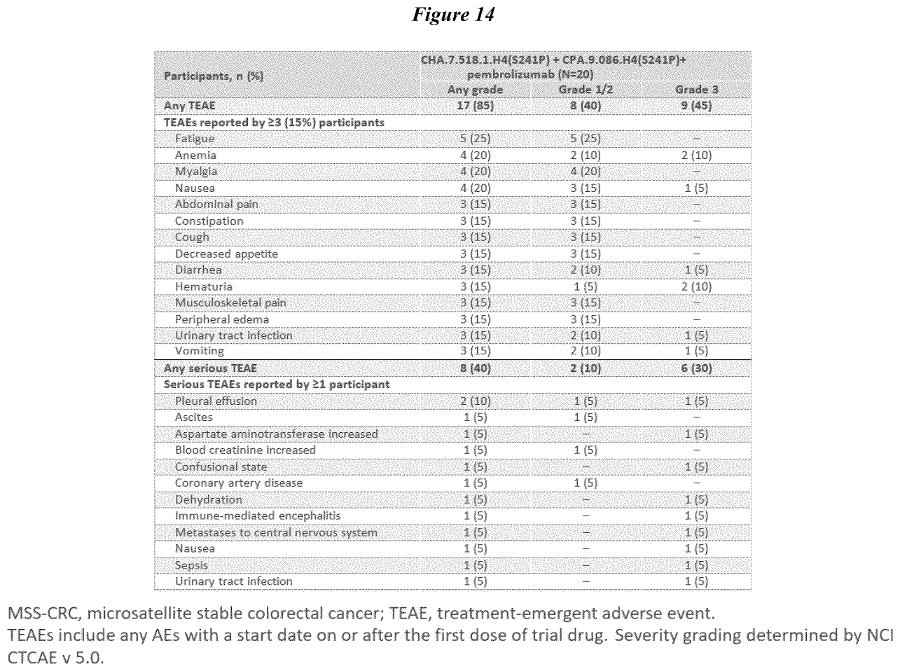

Treatment-emergent AEs (TEAEs) were reported in 17/20 (85%) participants (FIG. 14).

The most frequently reported TEAEs overall were fatigue, anemia, myalgia, and nausea, which are commonly reported in cancer patients treated with pembrolizumab and other approved PD-[L]1 inhibitors.

Most TEAEs were mild to moderate in severity (≤grade 2).

There were no grade 4 or 5 (life threatening or fatal) TEAEs.

Eight participants (40%) experienced ≥1 serious event.

Immune-mediated events were reported in 3 participants; all were considered possibly related to trial drugs:
One participant had a serious grade 3 immune mediated encephalitis, which was the only serious possibly related event.
One participant had grade 1 hyperthyroidism followed by grade 2 hypothyroidism.
One experienced a grade 1 rash.

### Anti-Tumor Activity

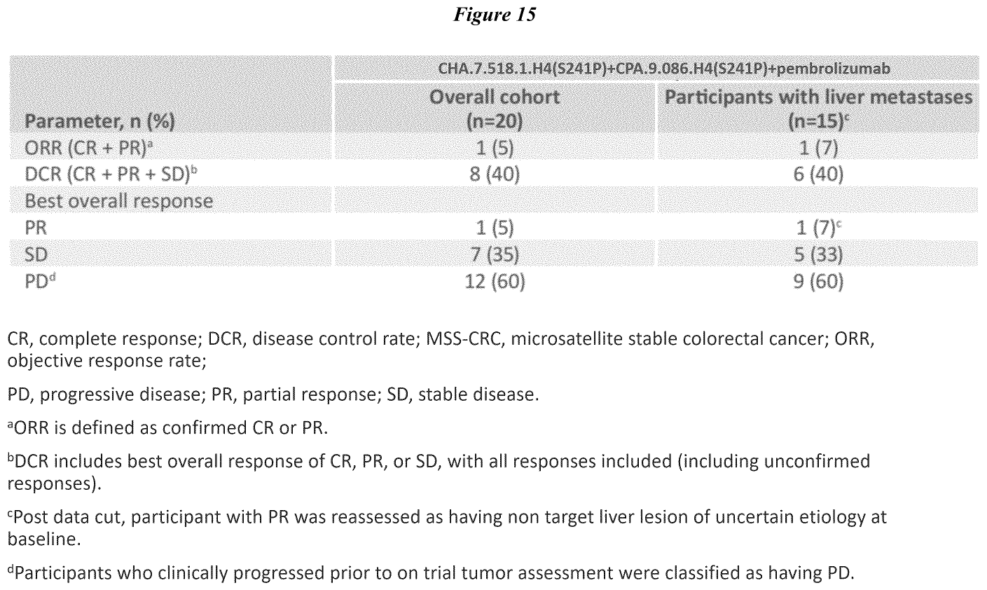

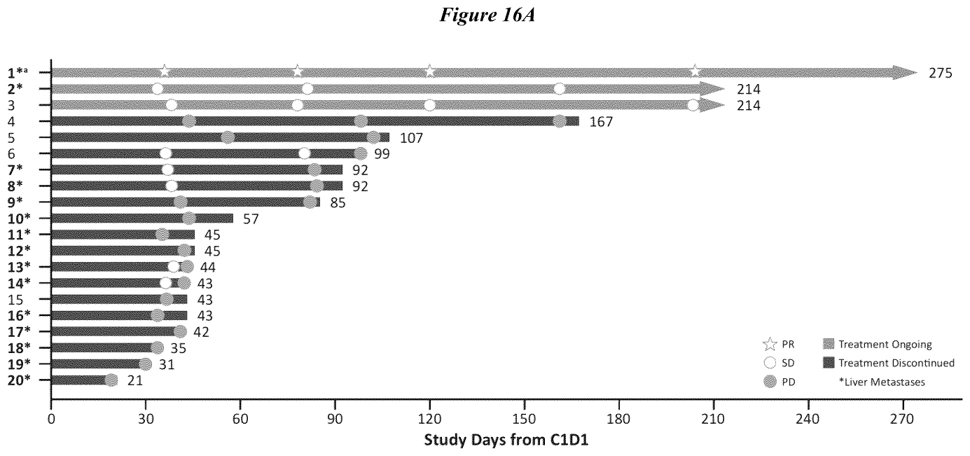

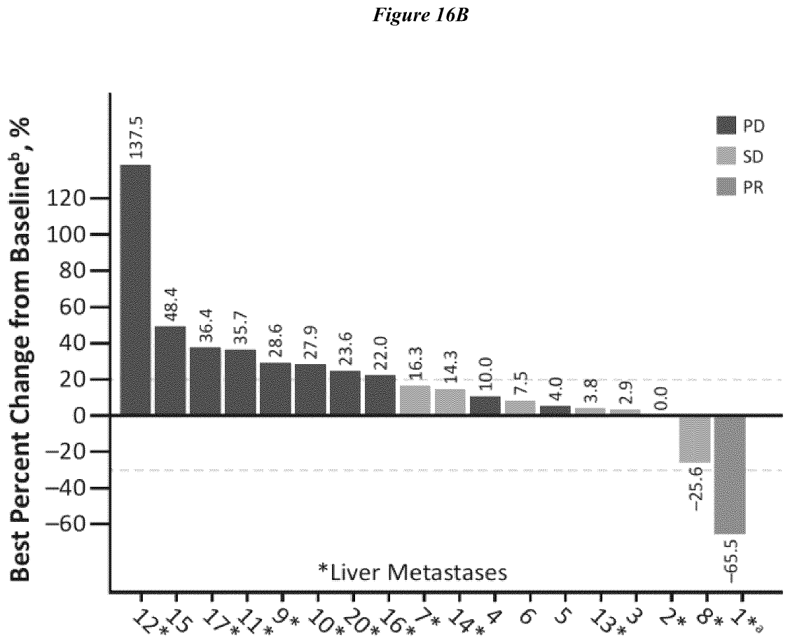

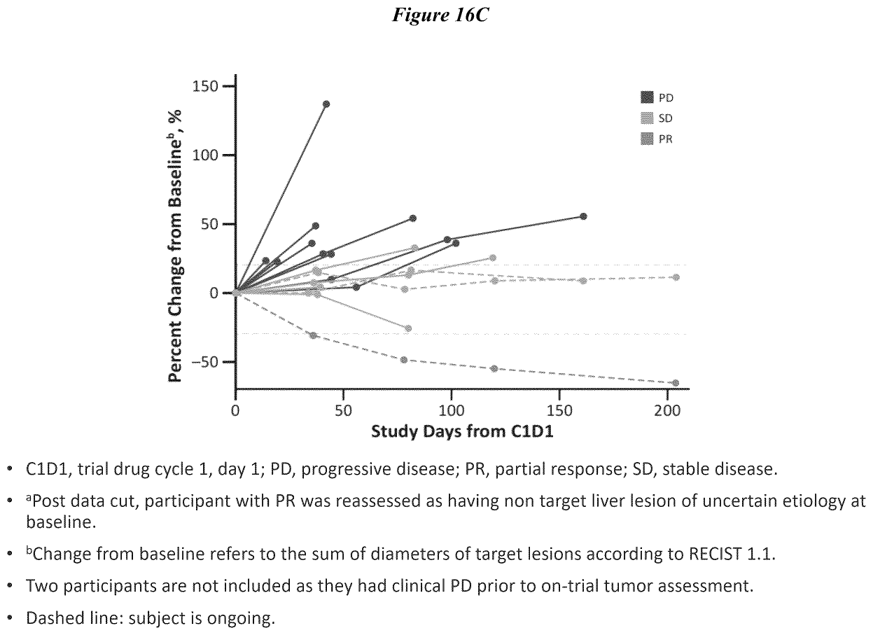

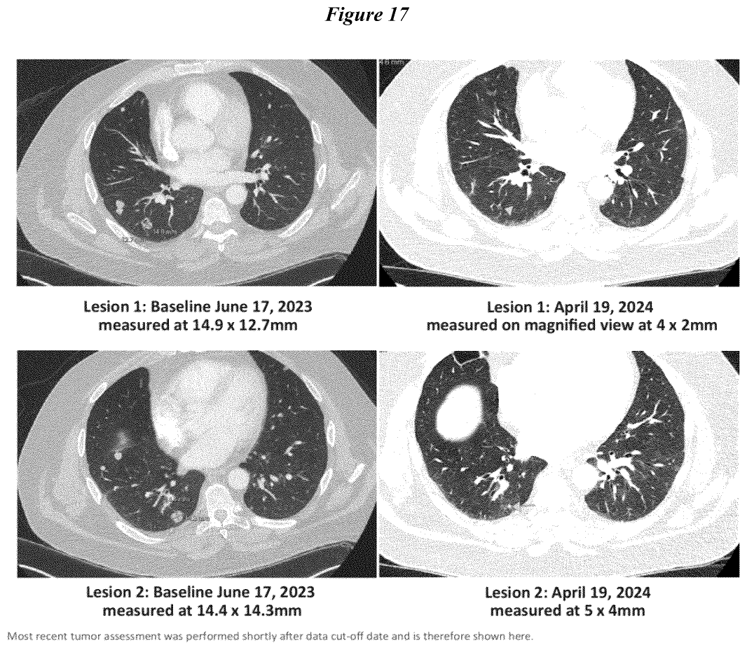

In both the overall cohort of participants with MSS-CRC and the subset of participants with liver metastases, the DCR was 40% (N= 8/20 and 6/15, respectively) (FIGS. 15-16).

One participant achieved a sustained partial response (see clinical vignette)

Clinical Vignette: Participant with a Partial Response

The subject is a 52 year old male, ECOG I with stage IV rectal cancer (histology: adenocarcinoma).
Initial diagnosis: 2016
Diagnosed stage IV: 2021
MSS KRAS negative.

At study entry the participant had target lesions in the lungs and a non target lesion in the liver which was reported as a hypo-dense lesion (liver lesion was reassessed to be of uncertain etiology).

The subject had 3 prior lines treatment, including multiple combinations of standard of care chemotherapy, panitumumab and bevacizumab.
The last regimen prior to trial entry was FOLFOX+bevacizumab (best response: progressive disease).

By time of data cutoff participant was on treatment and completed cycle 14.

### Pharmacodynamic Activation of the Immune System

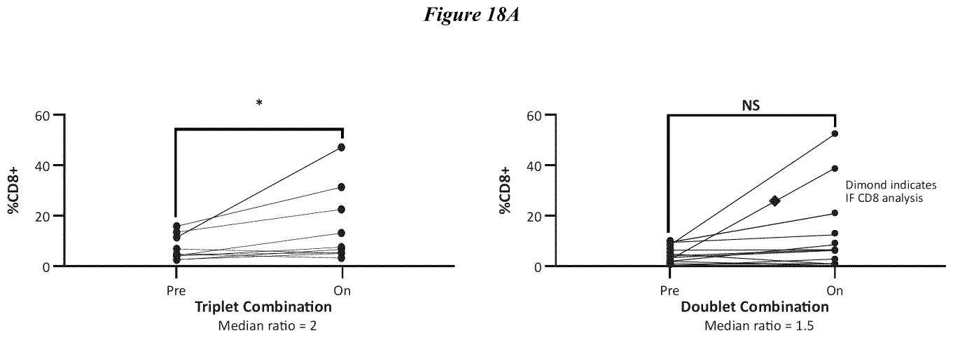

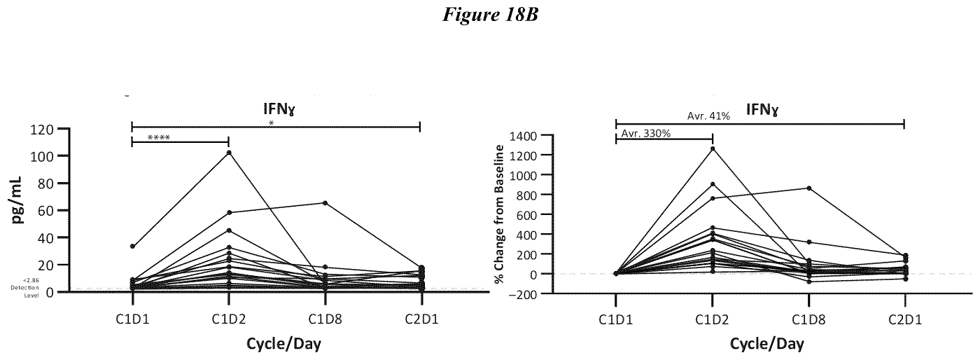

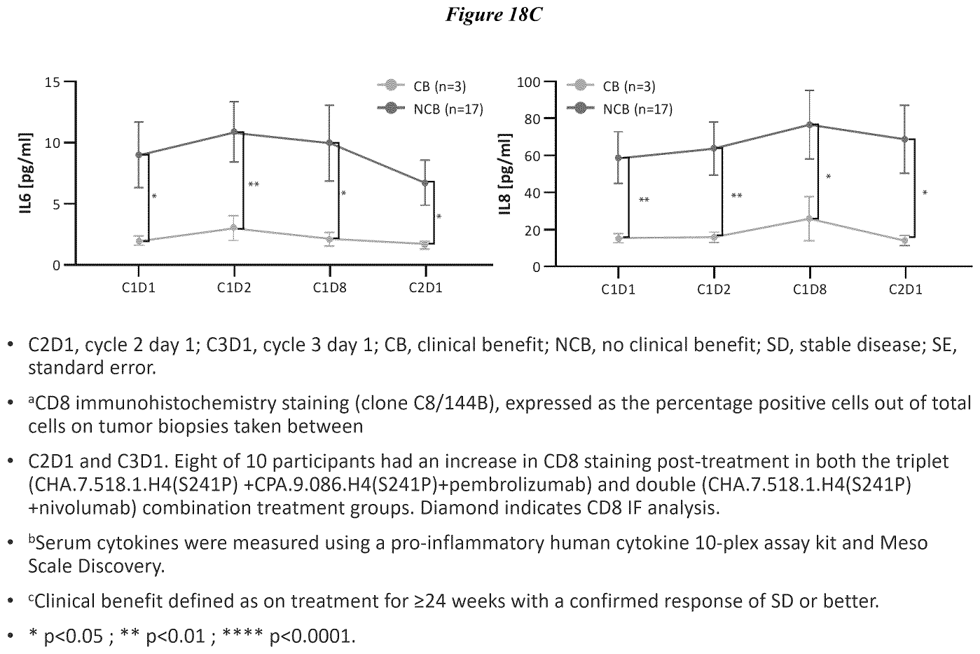

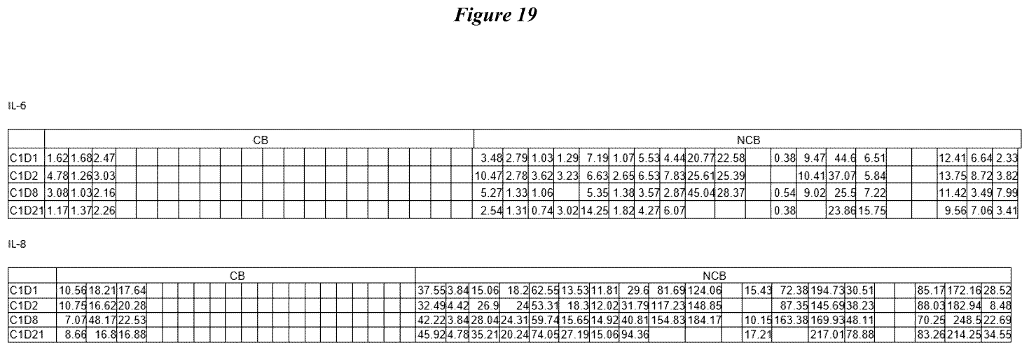

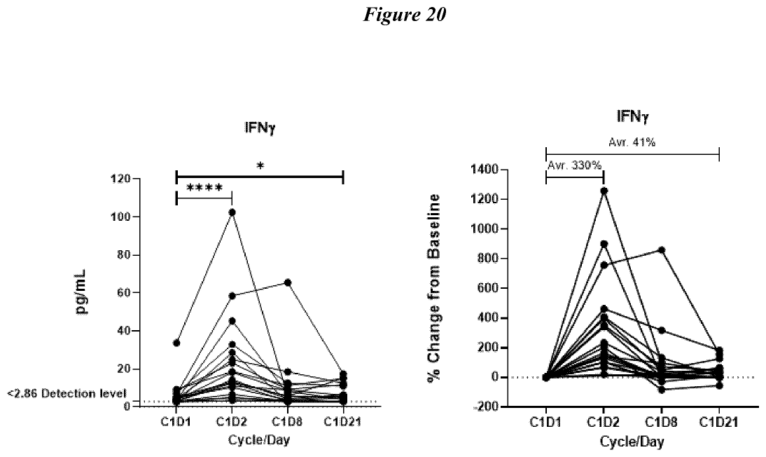

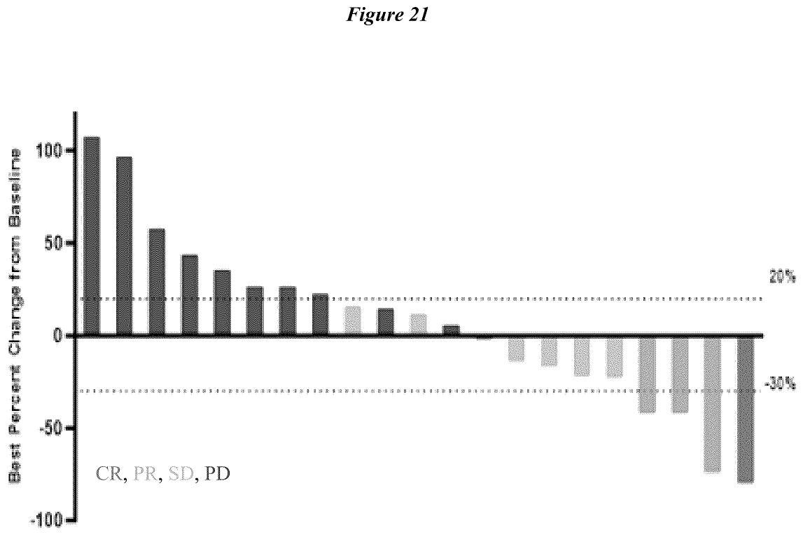

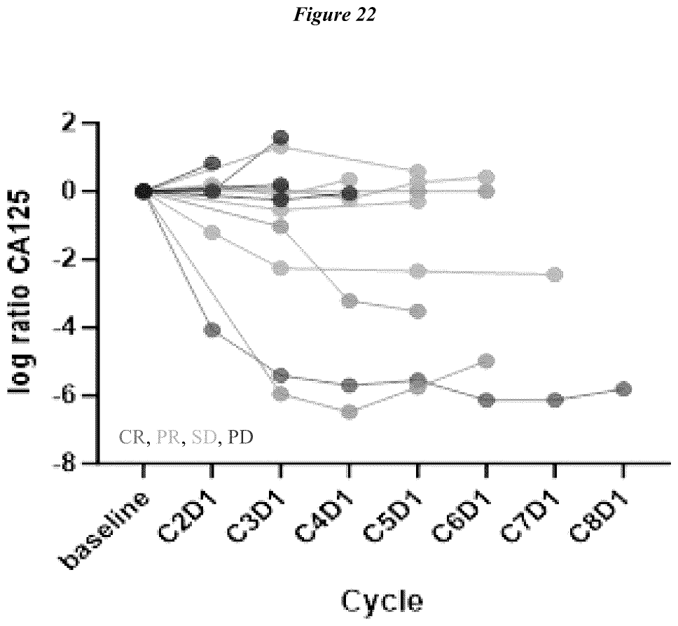

Increased CD8+ T cell infiltration was observed in tumors after treatment (FIG. 18A):
A median 2-fold increase in CD8+ T cell infiltration was observed after CHA.7.518.1.H4(S241P)+CPA.9.086.H4(S241P)+pembrolizumab, higher than with the doublet combination of CHA.7.518.1.H4(S241P)+nivolumab as reported from a previous trial. See, Rasco D, et al. J Immunother Cancer. 2022; 10(Suppl 2):A1-A1603.

Robust IFNγ induction was observed in patients' serum after treatment (FIG. 18B).

Significantly lower serum concentrations of immunosuppressive cytokines IL-6 and IL-8 were observed pre- and post-treatment among participants benefiting from treatment for more than 24 weeks (FIG. 18C).

### Conclusions

In this phase 1b expansion cohort of participants with heavily pre-treated advanced MSS-CRC, majority with liver metastases, the triplet combination of CHA.7.518.1.H4(S241P), CPA.9.086.H4(S241P) and pembrolizumab administered every 3 weeks was well tolerated. The safety profile of the triplet combination was generally consistent with that of pembrolizumab and other approved PD-L1 inhibitors.

Exploratory analysis demonstrated antitumor activity: ORR ( 1/20, 5%), DCR ( 8/20) 40%, best response of partial response in 1 patient with 3 patients (1PR, 2SDs) remaining on treatment at data cut.

Pharmacodynamic activation of the immune system in the tumor and blood was observed, supportive of CHA.7.518.1.H4(S241P) driven anti-tumor activity. Such activation was not previously reported in MSS-CRC with PD-L1 inhibitors.

Among the cohort reported here and in the prior reported data (see, Rasco D, et al. J Immunother Cancer. 2022; 10(Suppl 2):A1-A1603), it has been shown that CHA.7.518.1.H4(S241P) has a clinical benefit, is well tolerated and induced immune activation in a traditionally immune therapy resistant population of MSS-CRC. This data suggestive of CHA.7.518.1.H4(S241P)-driven effects in this resistant population may warrant further evaluation of other CHA.7.518.1.H4(S241P) drug combinations.

### Example 8: Triple Blockade of the DNAM-1 Axis with CHA.7.518.1.H4(S241P) (Anti-PVRIG)+Cpa.9.086.H4(S241P) (Anti-Tigit)+Pembrolizumab (Anti-Pd-1) Demonstrates Preliminary Antitumor Activity in Patients with Platinum Resistant OVCA, Interim Results of a Phase I Trial

### Background

Treatment [tx] options for platinum resistant ovarian cancer [PROC] are limited. Responses in this patient [pt] group with immune check point inhibitors [ICI], including combination with anti-TIGIT antibodies is <10% and ≈10% with chemotherapy^{1,2,3}. CHA.7.518.1.H4(S241P), a IgG4, potential 1′ in-class ICI blocks PVRIG, leading to activation of T-cells. CPA.9.086.H4(S241P) is an IgG4 ICI blocker of TIGIT. A 20% objective response rate (ORR) following combination CHA.7.518.1.H4(S241P)+BMS-986207 (anti-TIGIT)+nivolumab in PROC was previously reported³. In this example an additional cohort of PROC pts treated with CHA.7.518.1.H4(S241P), CPA.9.086.H4(S241P) and pembrolizumab demonstrating encouraging antitumor activity, and safety, consistent with the prior report is presented⁴.

### Methods

Clinical study is ongoing. The PROC cohort enrolled 25 pts. 25 safety-evaluable pts received CHA.7.518.1.H4(S241P)15 mg/kg+CPA.9.086.H4(S241P) 3 mg/kg+pembrolizumab 200 mg Q3W. Primary objectives [obj]: safety/tolerability; secondary obj: antitumor activity. Key inclusion criteria: Age ≥18 yrs, up to 3 prior lines of tx for histologically confirmed PROC. Key exclusion criteria: prior receipt of any ICI. Investigator assessed responses per RECIST v1.1, AE severity per CTCAE v5.0.

### Results

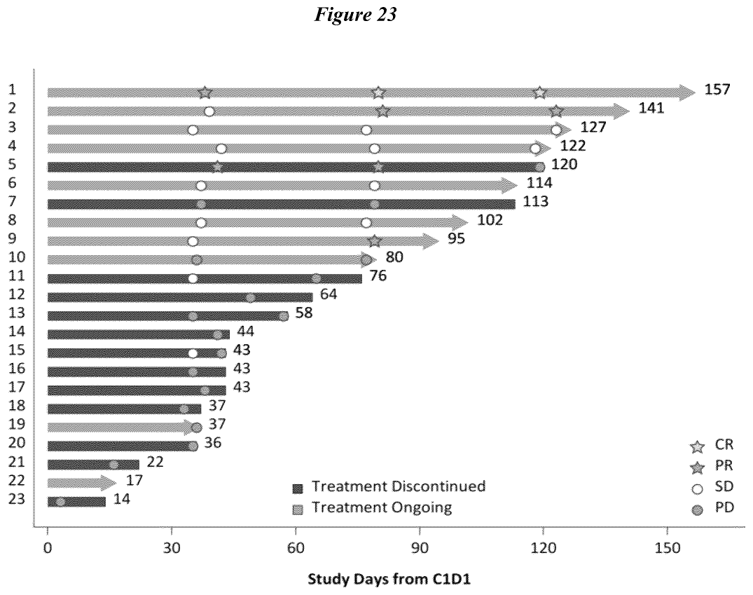

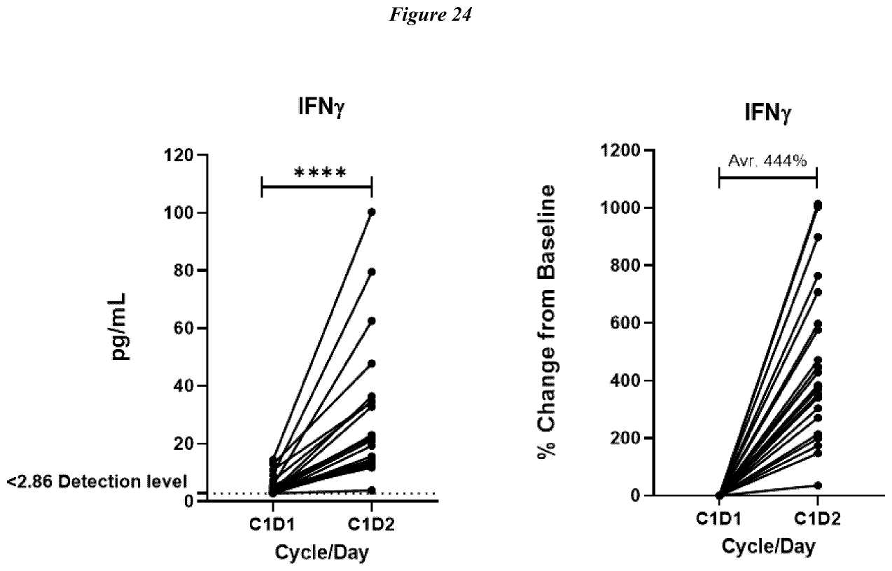

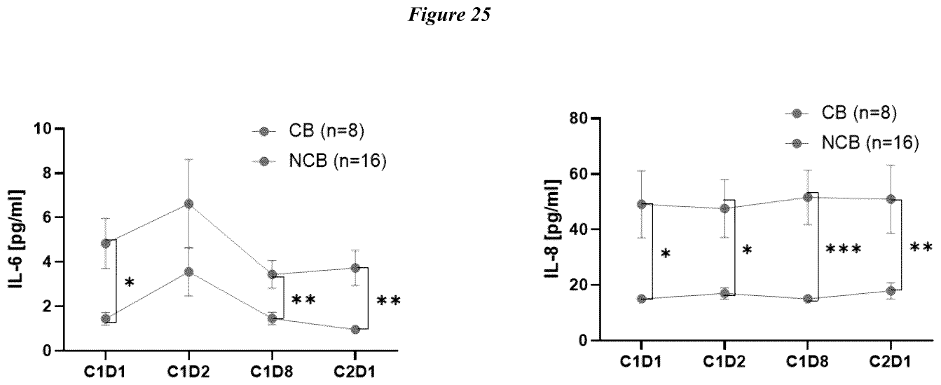

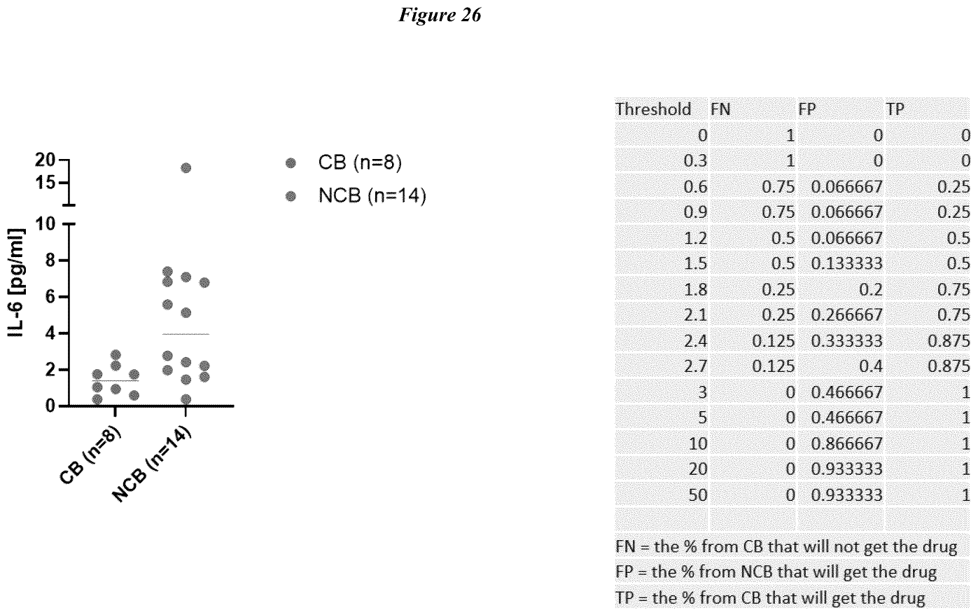

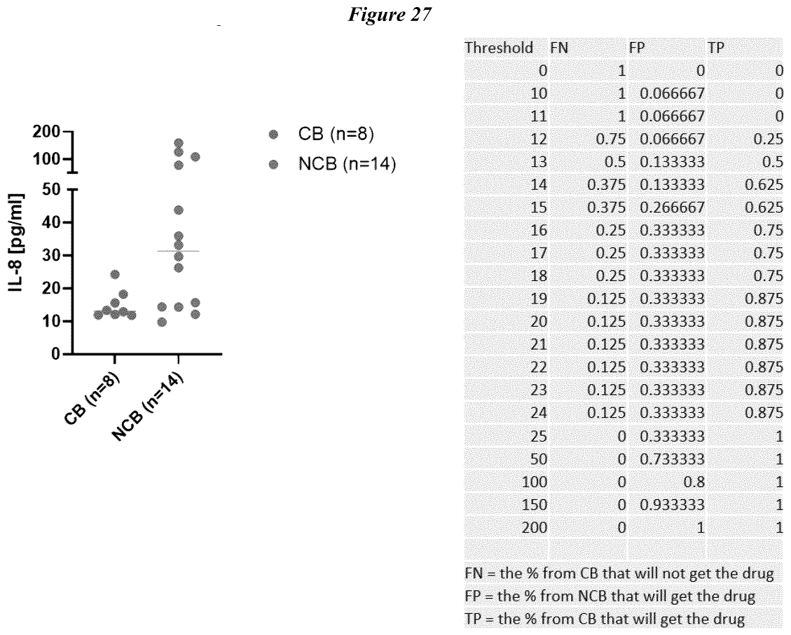

ORR was assessed in 23 efficacy evaluable pts median [med] age, 63yr, mean time on treatment 10.6 weeks [2-22 weeks, 10 pt ongoing, FIG. 23]. Best ORR 4/23 [17.4%] pts [1 CR, 3 PRs, Table 3] and 6 pts with SD, resulting in a disease control rate [CR+PR+SD] of 10/23 [43.5%, Table 3]. ADC treatment was given to 6 of the pts prior to enrollment on to the study. One PR pt had prior treatment with mirvetuximab soravtansine-gynx. Strong peripheral IFN-γ induction was observed following treatment in 14 out of 14 pts tested. Majority of AEs were of ≤2 grade in severity. No grade ⅘ events were reported. One grade 3 event of serious immune related encephalopathy was reported in one pt, which was resolving following treatment with steroids [Tables 3,4].

### Conclusions

The combination of CHA.7.518.1.H4(S241P)+CPA.9.086.H4(S241P)+pembrolizumab demonstrates encouraging signal of antitumor activity with immune activation in pts with heavily pre-treated PROC. The study further supports the clinical utility of blocking PVRIG and TIGIT both of the DNAM-1 pathway+anti-PD1 in PROC.

TABLE 2
Subject Disposition.

| | CHA.7.518.1.H4(S241P) + | CHA.7.518.1.H4(S241P) + |
|---|---|---|
| Variable | CPA.0.086.H4(S241P) + Pembro | CPA.9.086.H4(S241P) + Pembro |
| Reason | Safety Evaluable | Efficacy Evaluable |
| [n (%)]¹ | Population (N = 25) | Population (N = 23) |
| Ongoing in study treatment | 10 (40%) | 10 (43%) |
| Discontinued study treatment | 15 (60%) | 13 (57%) |
| Adverse Event | 1 (7%) | — |
| Progression per RECIST 1.1 | 12 (80%) | 11 (85%) |
| Progressive Disease due to | 2 (13%) | 2 (15%) |
| Clinical Evaluation | | |
| Ongoing in study | 21 (84%) | 19 (83%) |
| Discontinued study | 4 (16%) | 4 (17%) |
| Death | 3 (75%) | 3 (75%) |
| Other | 1 (25%) | 1 (25%) |

¹Denominator for each reason is the number of patients who discontinued study (treatment) in the respective dose group.

TABLE 3
Summary of Overall Response Rate (ORR)
and Disease Control Rate (DCR).

| | | CPA.9.086.H4(S241P) + |
|---|---|---|
| | | CHA.7.518.1.H4(S241P) + |
| | Characteristics | Pembro (N = 23) |
| ORR (CR + PR)¹ | | 4 (179%) |
| DCR (CR + PR + SD)² | | 10 (43%) |
| Best Overall | | |
| Response (BOR) | | |
| | CR | 1 (4%) |
| | PR | 3 (13%) |
| | SD | 6 (26%) |
| | PD | 12 (52%) |
| | Missing³ | 1 (4%) |

CR, complete response; PR, partial response; SD, stable disease; PD, progressive disease; ORR, objective response rate; DCR, disease control rate.
¹Unconfirmed assessments included - 1 PR.
²Unconfirmed assessments included - 1 PR, 2 SD.
³Firstpost screening tumor assessment not yet performed by time of data cutoff

TABLE 4
Overall Summary of TEAEs ovarian cancer, triplet dose expansion.

| | CPA9.086.H4(S241P) + |
|---|---|
| Adverse Event | CHA.7.518.1.H4(S241P) |
| Category [n (%)] | Pembro (N = 25) |
| Any TEAE | 25 (100%) |
| Any Grade 3 or Greater TEAEs | 7 (28%) |
| Any Serious TEAE | 4 (16%) |
| Any Serious Related TEAEs* | 1 (4%) |
| Any TEAEs Leading to | 2 (8%) |
| Treatment Discontinuation | |
| Any Infusion-Related TEAEs | 3 (12%) |
| Any Immune-Related TEAEs | 11 (44%) |
| Any TEAE leading to Death | 0 (0%) |

TEAE—Treatment emergent adverse event

TABLE 5
TEAEs by Preferred Term and Severity reported in at least 15%
of the participants, Ovarian Cancer, triplet dose expansion.

| AE preferred | | | | | |
|---|---|---|---|---|---|
| Term/n [%] | Grade | Grade | Grade | Grade | All |
| (N = 25) | 1 | 2 | 3 | 4/5 | Grades |
| Any TEAE | 7 (28) | 11 (44) | 7 (28) | — | 25 (100) |
| Fatigue | 4 (16) | 3 (12) | 1 (4) | — | 8 (32) |
| Diarrhoea | 6 (24) | 1 (4) | — | — | 7 (28) |
| Nausea | 3 (12) | 3 (12) | — | — | 6 (24) |
| Abdominal | 1 (4) | 3 (12) | — | — | 4 (16) |
| pain | | | | | 4 (16) |
| Vomiting | 4 (16) | — | — | — | |

### REFERENCES

1. Zhang Y, et al. Current Advances in PD-1/PD-L1 Blockade in Recurrent Epithelial Ovarian Cancer. Front Immunol 2022; 13:901772.
2. Perets R, et al. Safety and efficacy of vibostolimab (vibo) plus pembrolizumab (pembro) and coformulation of vibo/pembro in ovarian cancer naive to PD-1/PD-L1 inhibitors. Cancer Research. 2022; 82(suppl 12; abstract CTI80).
3. Mutch D G, et al. Randomized phase III trial of gemcitabine compared with pegylated liposomal doxorubicin in patients with platinum-resistent ovarian cancer. J Clin Oncol. 2007; 25(19):2811.
4. Moroney J, et al. Triple blockade of the DNAM-axis with COM701+BMS-986207+nivolumab demonstrates preliminary antitumor activity in patients with platinum resistant OVCA. Immuno-Oncology and Technology 2022; 16 (suppl 1; abstract 158P).

### Example 9: Triple Blockade of DNAM-1 Axis with CHA.7.518.1.H4(S241P) (Anti-PVRIG)+Cpa.9.086.H4(S241P) (Anti-Tigit)+Pembrolizumab Shows Preliminary Antitumor Activity in Patients with Platinum Resistant Ovarian Cancer, Interim Results of a Phase I Trial

### INTRODUCTION

Ovarian cancer is the 5th leading cause of cancer-related death among women and a leading cause of death among all gynecological cancers. (Ovarian Cancer Research Alliance, 2024, https://ocrahope.org/get-the-facts/statistics/.)

Platinum-resistant ovarian cancer (PROC) represents a challenging population to treat with a high unmet need for new treatments.

The overall response rate (ORR) in patients with PROC treated with anti-PD-(L)1+/−anti-TIGIT is approximately 10%. (Zhang Y, et al. Front Immunol. 2022; 13:901772. Perets R, et al. Cancer Res. 2022; 82(suppl 12; abstract CT180). Ghisoni E, et al. Nat Rev Clin Oncol. 2024; Epub ahead of print.)

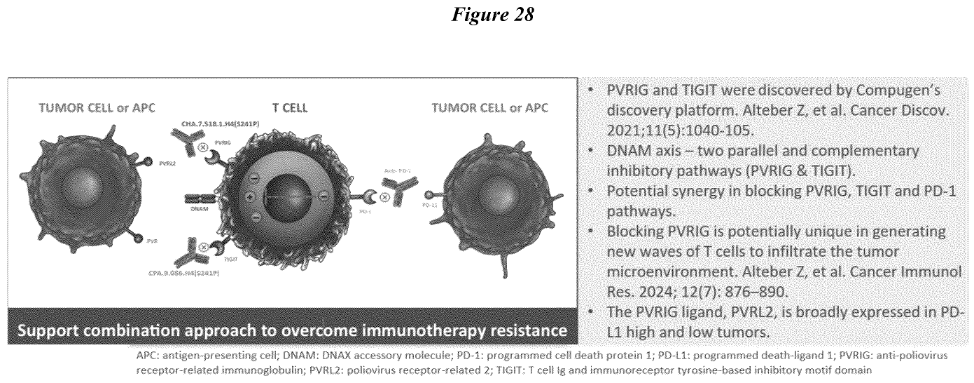

CHA.7.518.1.H4(S241P) is a potential 1st-in-class immune checkpoint inhibitor (ICI) that inhibits the DNAM-1 axis member PVRIG, leading to the activation of T and natural killer (NK)-cells (FIG. 28). CPA.9.086.H4(S241P) is a potential best-in-class ICI that inhibits the DNAM-1 family member TIGIT. (Hansen K, et al. Cancer Immunol Immunother. 2021; 70(12):3524-3540.)

In a prior Phase I clinical trial, the combination of CHA.7.518.1.H4(S241P)+BMS-986207 (anti-TIGIT)+nivolumab (anti-PD1) demonstrated an ORR of 4/20 (20%) in PROC patients. (Gaillard et al., J Immun Ther Cancer. 2023; 11: doi: 10.1136/jitc-2023-SITC2023.0669.)

It is hypothesized that a triplet combination immunotherapy of CHA.7.518.1.H4(S241P)+CPA.9.086.H4(S241P)+pembrolizumab (anti-PD1) would demonstrate antitumor activity by inhibiting the DNAM-1 axis in PROC which would have a favorable safety and tolerability profile.

### Objectives

Provided herein are preliminary results of a cohort of PROC patients treated with the triplet combination of CHA.7.518.1.H4(S241P)+CPA.9.086.H4(S241P)+pembrolizumab in existing clinical trial demonstrating encouraging safety and antitumor activity, consistent with a prior PROC cohort. (Gaillard et al., J Immun Ther Cancer. 2023; 11: doi: 10.1136/jitc-2023-SITC2023.0669.)

### Key Primary Objective

To evaluate the safety profile of
CHA.7.518.1.H4(S241P)+CPA.9.086.H4(S241P)+pembrolizumab.

### Prespecified Exploratory Objectives

Preliminary antitumor activity of
CHA.7.518.1.H4(S241P)+CPA.9.086.H4(S241P)+pembrolizumab.

Pharmacodynamic effects of trial drug(s) in the tumor and blood, including changes in serum cytokine levels.

### Methods

### Trial Design

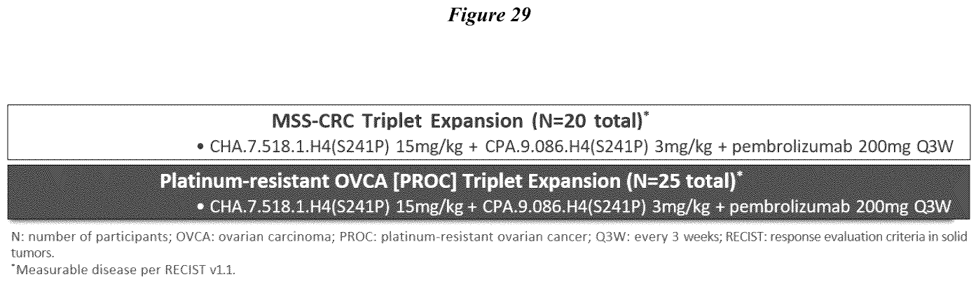

Data reported are from the PROC expansion cohort of an ongoing Phase I, open-label, multicenter, dose-escalation, and expansion trial that is being conducted in the US (FIG. 29).

### Participants

### Key Inclusion Criteria:

Advanced epithelial ovarian, fallopian tube, or primary peritoneal carcinoma.

PROC defined as disease recurrence <6 months after completion of a platinum-containing regimen.

Patients with primary platinum-refractory disease (defined as progression of disease prior to completion of 1st line platinum therapy or immediately following [≤3 months following last date of chemotherapy]) were ineligible.

Patients will have received ≤3 prior lines for PROC.

Baseline organ function: creatinine clearance ≥40 mL/min (Cockcroft and Gault equation), albumin ≥3.0 g/dL, hemoglobin ≥8 g/dL, and platelet count ≥50,000/mm3.

### Key Exclusion Criteria:

Prior treatment with a TIGIT inhibitor, PVRIG inhibitor, anti-PD-1/PD-L1/2, anti-CD96 antibody, anti-OX-40 antibody, anti-CD137 antibody, anti-LAG3, anti-TIM3, anti-CTLA4 antibody.

History of another malignancy within 2 years prior to first study drug administration.

### Treatment

Study treatment up to 35 cycles (˜24 months) until progressive disease, unacceptable toxicity, initiation of new anticancer therapy, withdrawal of consent, or death.

### Safety and Response Evaluation

Antitumor activity is evaluated per RECIST v1.1 with tumor assessments performed every 6 weeks for the first 6 cycles and every 12 weeks thereafter or at any time point where PD is suspected.

Blood inflammatory markers (i.e., serum cytokines) including IFN-γ, IL-6 and IL-8 levels are tested on C1D1 pre-infusion, C1D2, C1D8, and C1D21 (i.e., C2D1 pre-dose).

Safety is assessed throughout the study until 90 days post last dose of study treatment.

### Results

### Participant Baseline Demographics and Disease Characteristics

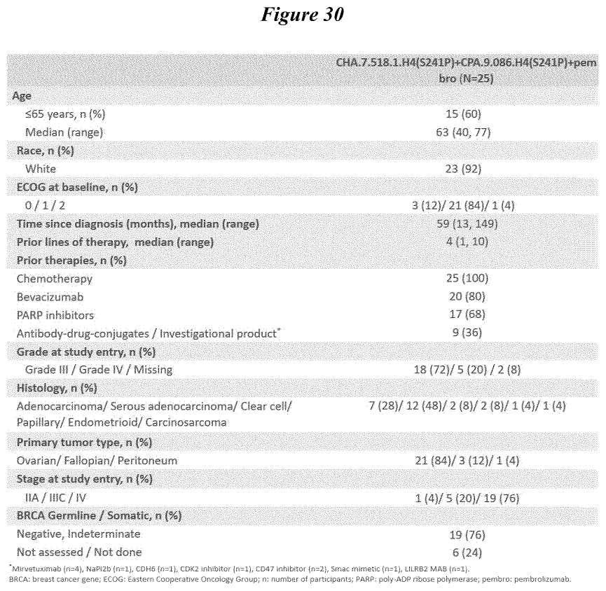

At data cut-off date, 25 participants were enrolled and treated. Baseline characteristics are summarized in FIG. 30.

The majority of the participants had high-grade adenocarcinoma/serous adenocarcinoma. Papillary and clear cell carcinoma were present in 2 participants (each) and endometrioid and carcinosarcoma each in 1 participant.

The patient population was heavily pretreated (median: 4 prior lines of therapy).

Majority of participants were treated with bevacizumab and PARP inhibitors prior to enrollment into the study.

More than a third of participants (n=9), were previously treated with antibody-drug conjugates (ADC) (n=6) and/or other investigational products (n=5). Two participants were treated with both.

### Participant Disposition and Exposure

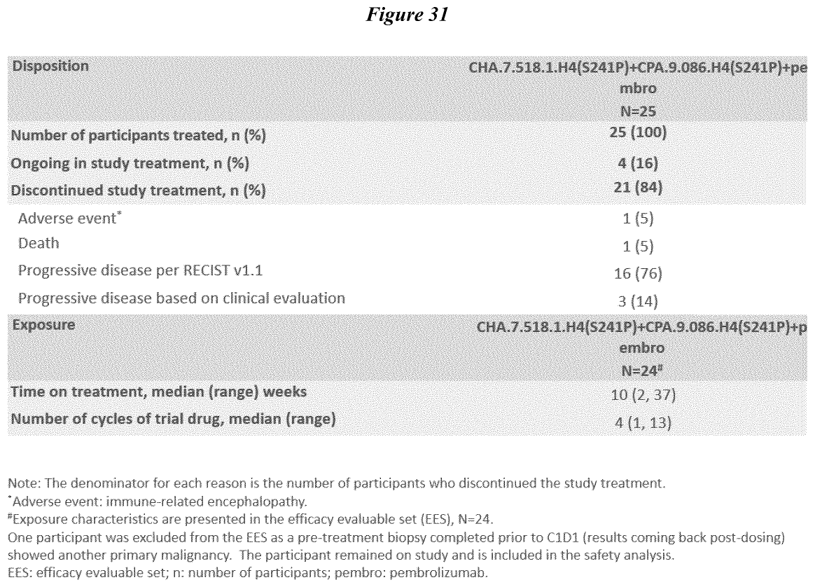

At data cut-off date, 21 of the 25 enrolled participants (84%) had discontinued trial treatment: 1 participant due to an AE, 1 due to death, and 19 participants due to disease progression (FIG. 31).

The trial is ongoing; 4 (16%) participants remain under treatment.

### Safety

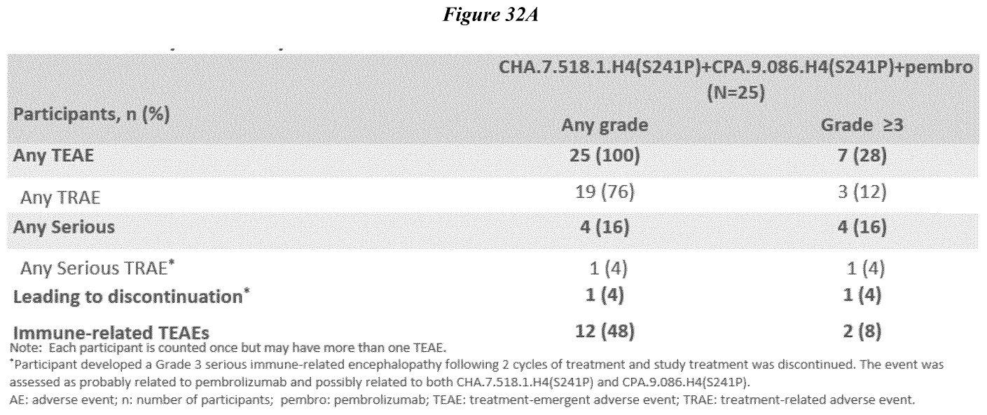

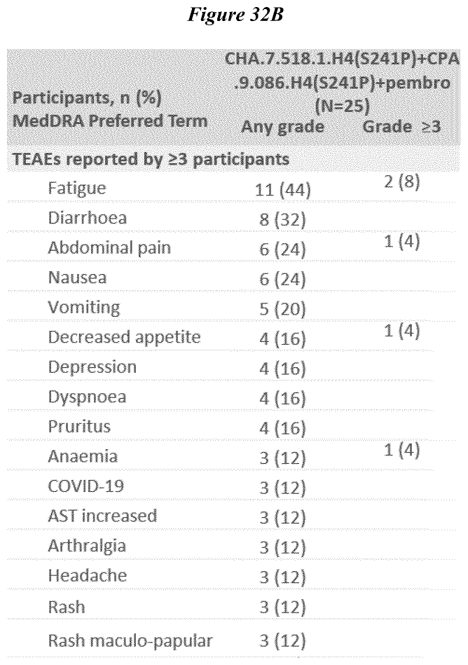

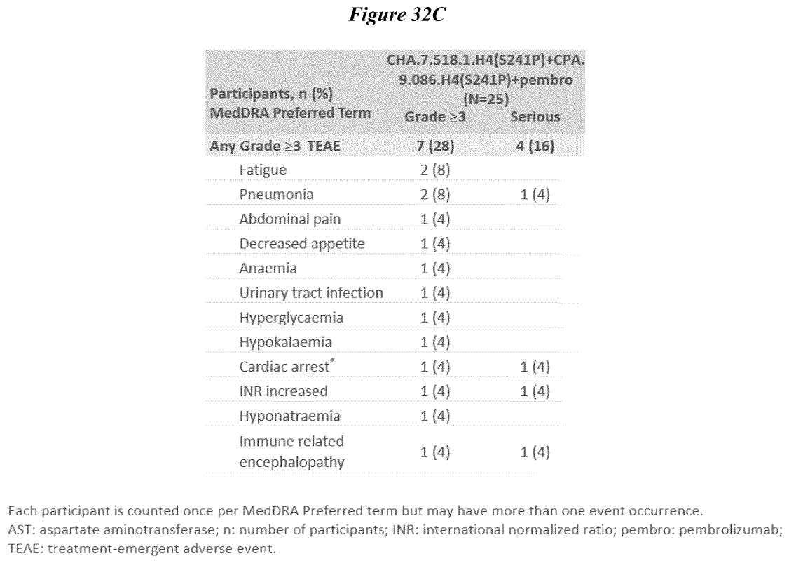

Treatment-emergent adverse events (TEAEs) were reported in all 25 participants (100%) (FIG. 32 3A).

Most TEAEs were mild to moderate in severity (≤Grade 2).

Grade ≥3 TEAEs were reported in 7 participants (28%) (FIG. 32B).

One Grade 5 SAE of cardiac arrest was reported, assessed as not related to study treatment (FIG. 32C).

One Grade 3 serious immune-related encephalopathy was reported (FIG. 32C):

The participant improved following treatment with steroids.

Treatment with all 3 study drugs was discontinued.

### Antitumor Activity

24 of the 25 treated participants were evaluable for response.

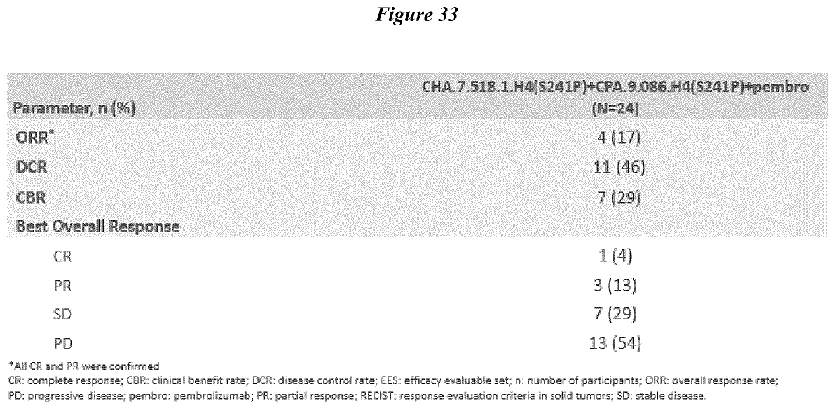

ORR (CR+PR) was 4/24 (17%) and DCR (BOR: CR+PR+SD) was 11/24 (46%) (FIG. 33).

The clinical benefit rate (CBR: confirmed CR/PR or SD for >180 days) was 7/24 (29%) (FIG. 33).

Among the 7 participants with CBR, 2 received prior treatment with ADC and 1 with Smac mimetic.

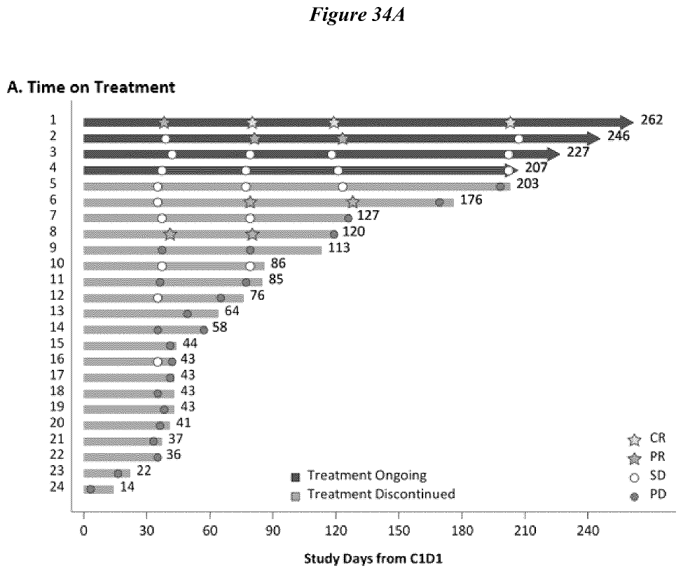

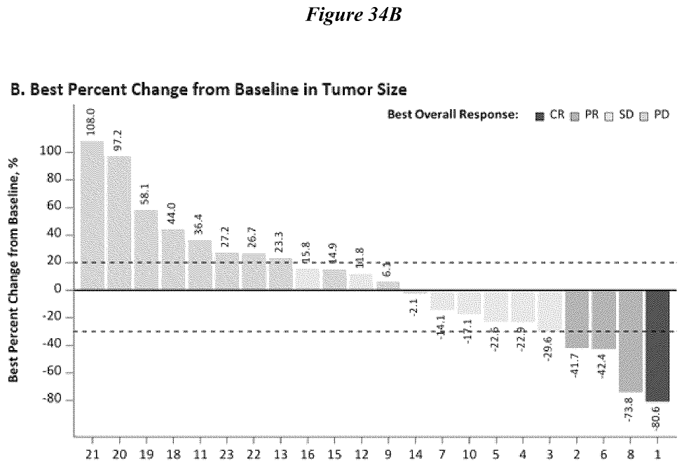

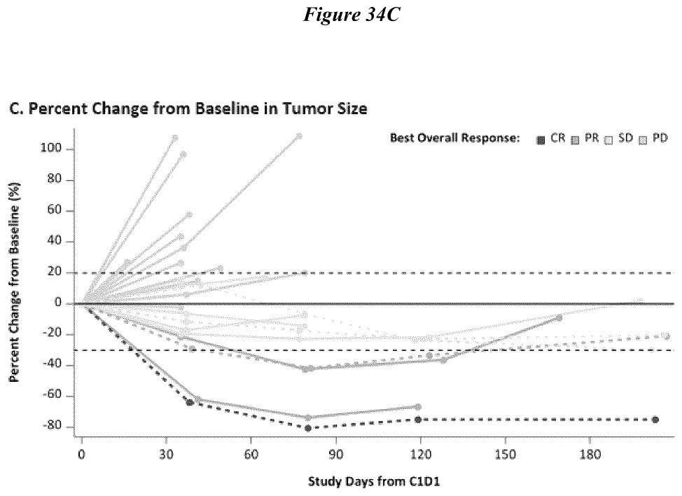

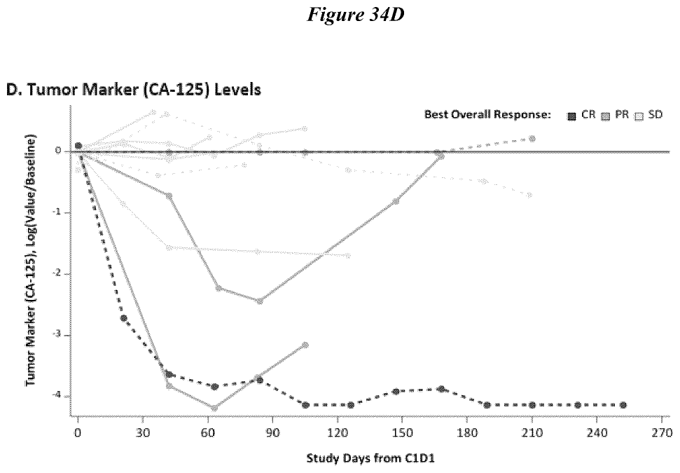

Four participants (BOR of 1 CR, 1 PR, 2 SDs) continued treatment for >200 days (FIG. 34A).

One of the 2 SDs (FIG. 34B, Participant #3) presented with a best target lesion response reduction of 29.6% in the last tumor assessment.

Reductions in CA-125 were observed in 3 of the SD participants, supporting the observed benefit in these participants (FIG. 34D).

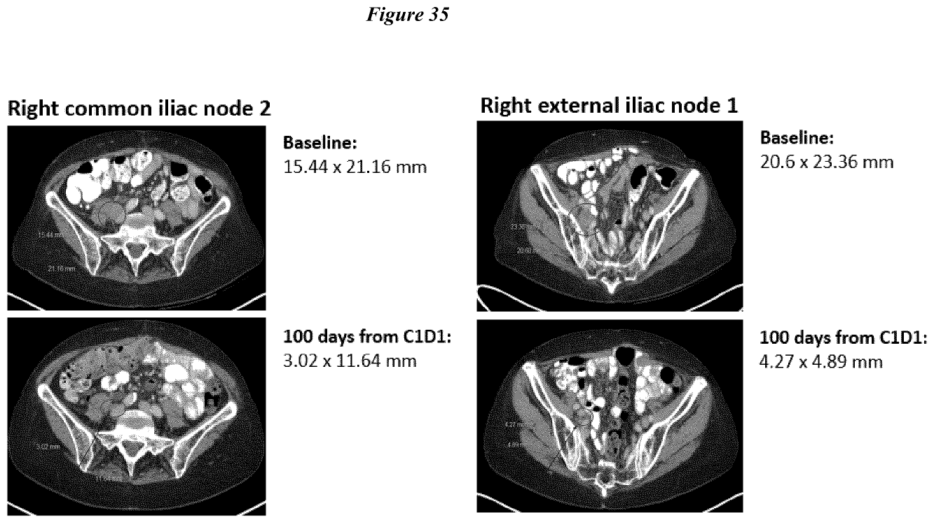

Clinical Vignette: Participant with a Complete Response (FIG. 35)

69 year-old patient with baseline ECOG 1 and stage IV PROC (histology: grade III Serous adenocarcinoma).

Initial Diagnosis: 2018; Stage IV diagnosed: 2022.

Prior lines of treatment included combinations of carboplatin and Taxol/Nab-paclitaxel and bevacizumab maintenance.

The last treatment line in a clinical trial with an investigational product (Smac mimetic) with the best response of SD.

As of the data cut-off date, the participant was still on treatment (C13).

### Pharmacodynamic Activation of the Immune System

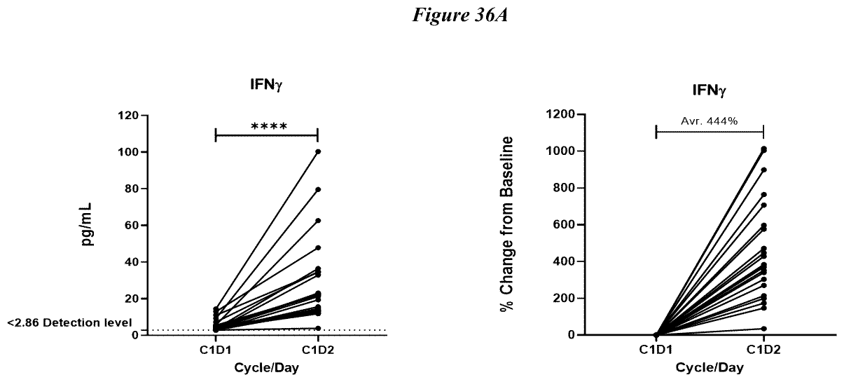

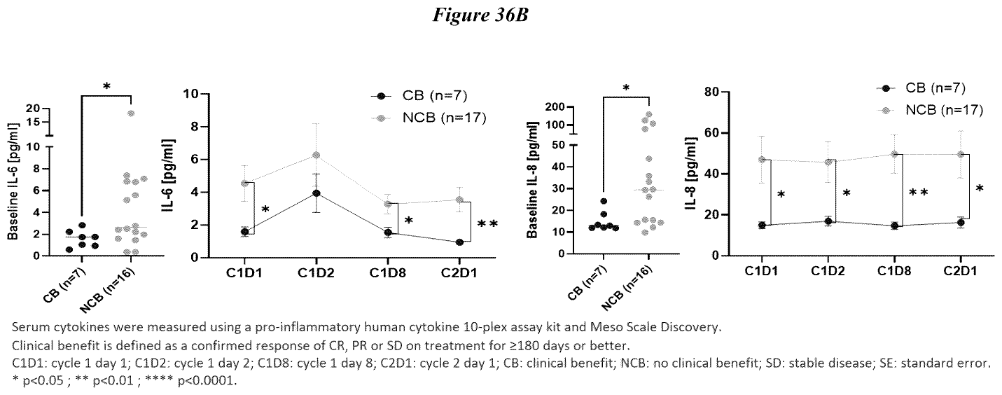

Statistically significant peripheral IFN-γ induction was observed at C1D2, one day after treatment with CHA.7.518.1.H4(S241P)+CPA.9.086.H4(S241P)+pembrolizumab, compared with C1D1 (pre-treatment) (FIG. 36A).

Significantly lower serum concentrations of immunosuppressive cytokines IL-6 and IL-8 were observed among participants benefiting from treatment, both pre-treatment and during treatment (FIG. 36B).

### Conclusions

This ongoing Phase I expansion cohort confirms previous findings showing encouraging efficacy and safety of triple blockade of the DNAM-1 axis in advanced heavily pre-treated PROC patients, where 36% of the population had been on other investigational agents prior to study entry.

The safety and tolerability profile was generally consistent with approved anti-PD-(L)1s.

Clinical benefit, with deep and durable responses, was observed in patients with high-grade adenocarcinoma, in a patient with carcinosarcoma—historically nonresponsive to ICIs, and in patients previously exposed to ADCs and other investigational drugs:
ORR 17%, DCR 46%, CBR 29%, with 4 patients still on treatment at data cut.

Robust pharmacodynamic activation of the immune system was observed on treatment.

Lower concentrations of immunosuppressive cytokines prior to and on treatment were associated with a clinical benefit.

The totality of the data from both cohorts supports the use of the triplet blockade of PVRIG, TIGIT, and PD-1 in ovarian cancer, with the potential to expand beyond PROC into earlier settings.

### CITED REFERENCES

1. Ovarian Cancer Research Alliance, 2024, https://ocrahope.org/get-the-facts/statistics/.
2. Zhang Y, et al. Front Immunol. 2022; 13:901772.
3. Perets R, et al. Cancer Res. 2022; 82(suppl 12; abstract CT180).
4. Ghisoni E, et al. Nat Rev Clin Oncol. 2024; Epub ahead of print.
5. Hansen K, et al. Cancer Immunol Immunother. 2021; 70(12):3524-3540.
6. Gaillard et al., J Immun Ther Cancer. 2023; 11: doi: 10.1136/jitc-2023-SITC2023.0669.
7. Alteber Z, et al. Cancer Discov. 2021; 11(5):1040-105.
8. Alteber Z, et al. Cancer Immunol Res. 2024; 12(7): 876-890.

### Example 10: Anti-Tumor Activity and Safety Data of CHA.7.518.1.H4(S241P) (Anti-PVRIG), Cpa.9.086.H4(S241P) (Anti-Tigit) and Pembrolizumab Combination in Patients with Platinum Resistant Ovarian Cancer

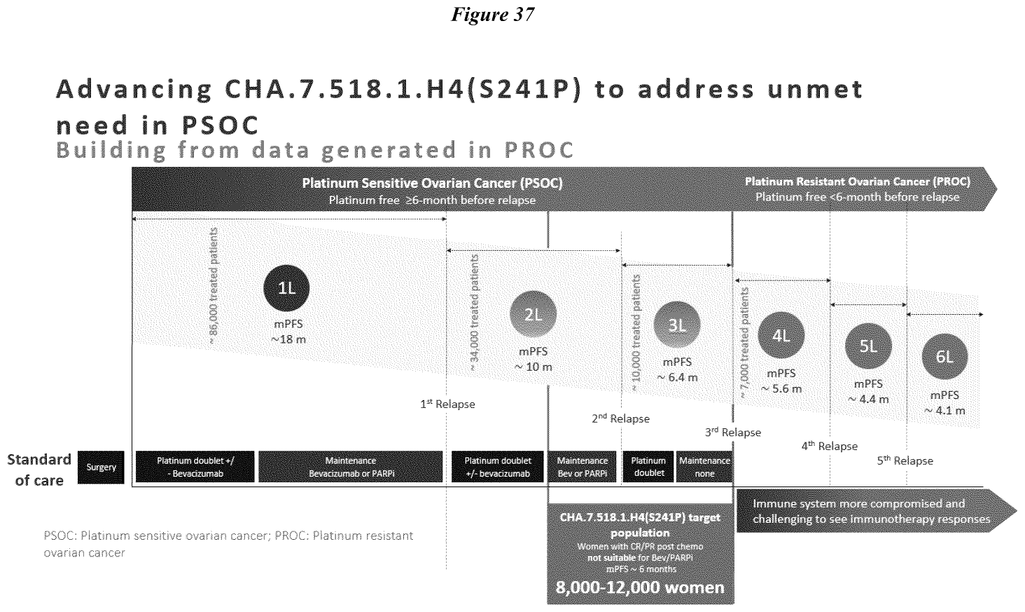

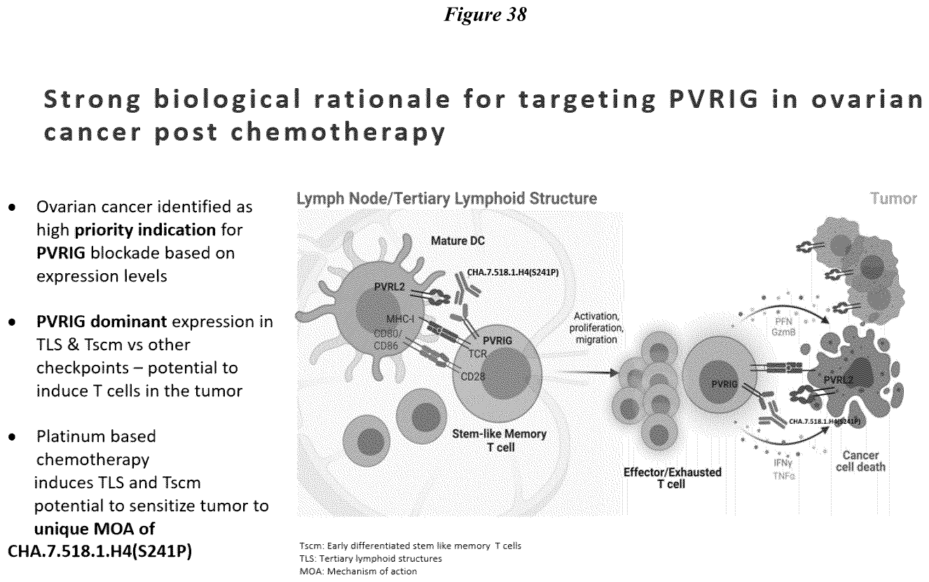

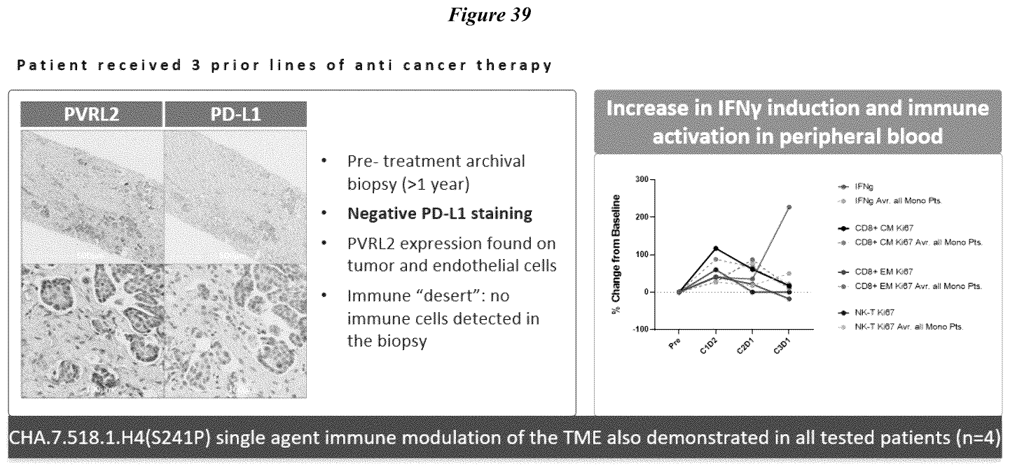

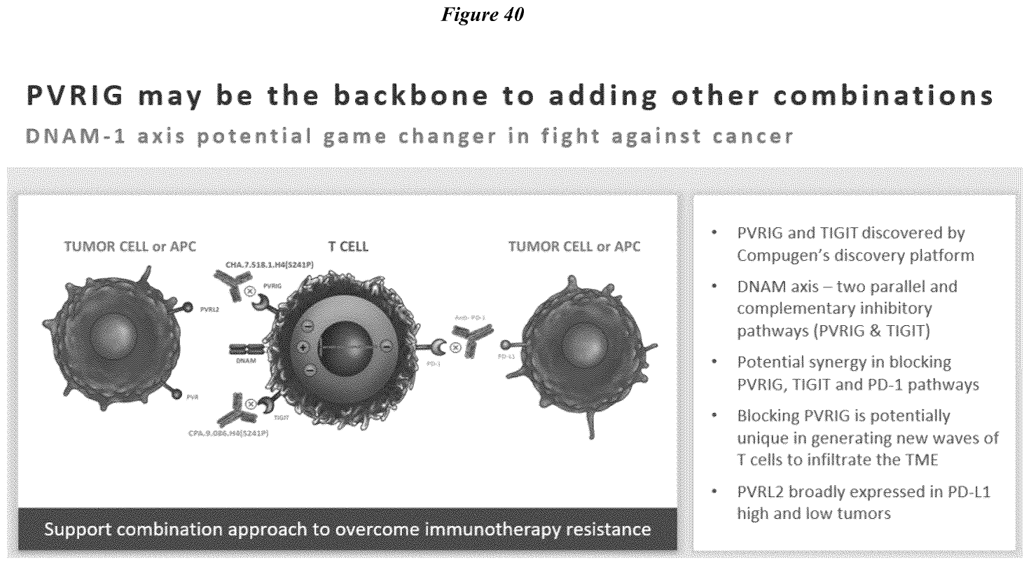

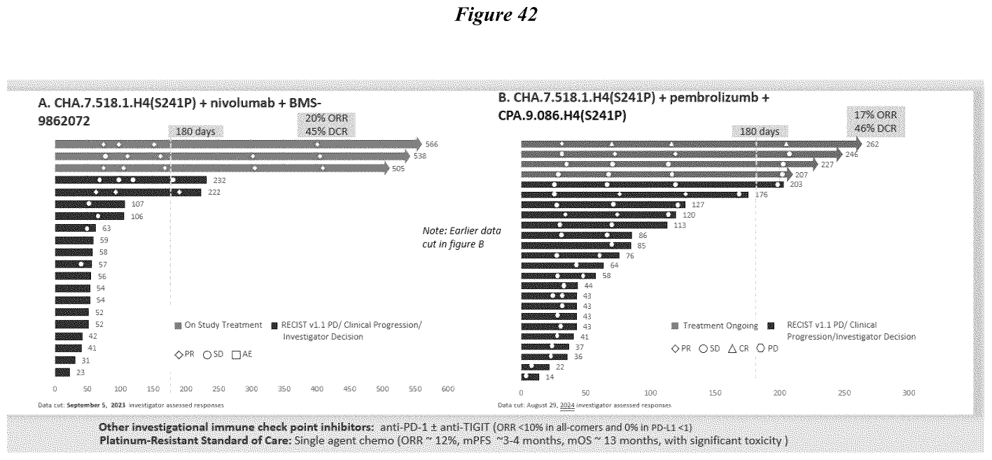

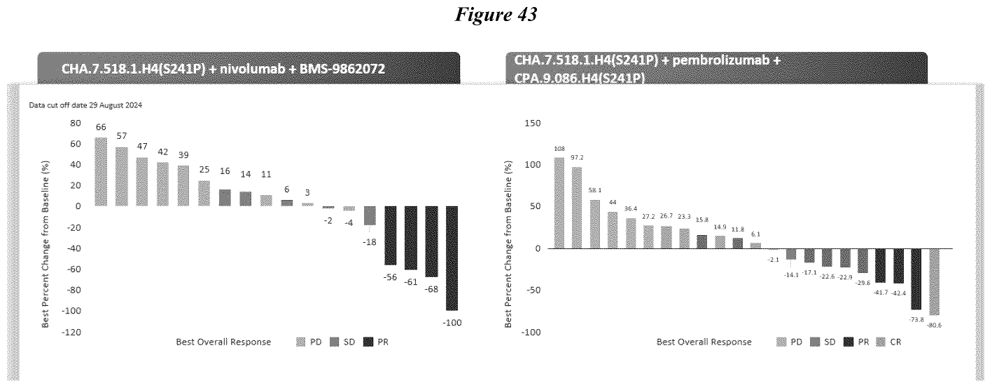

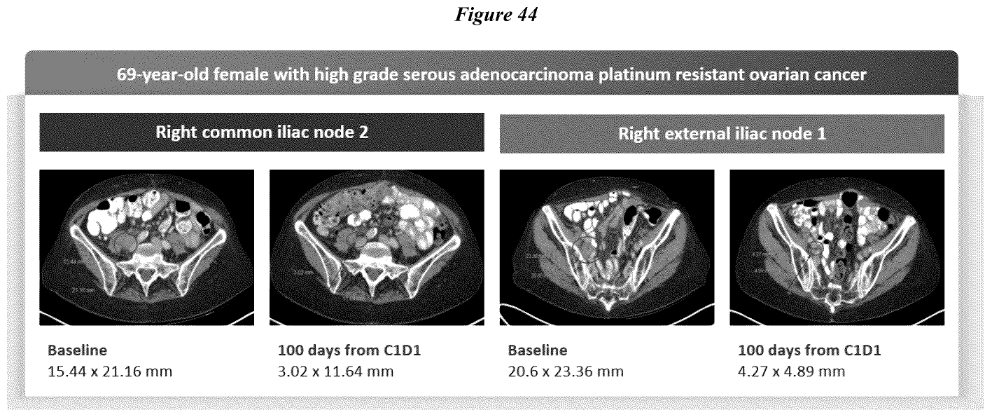

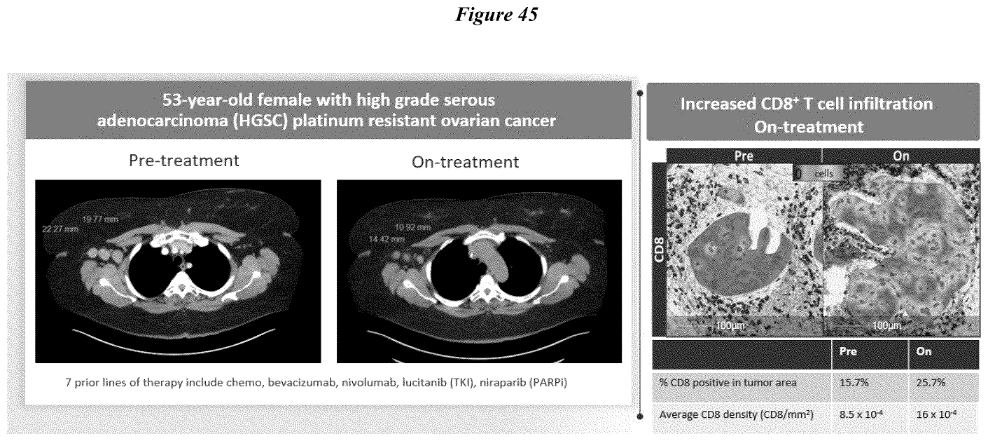

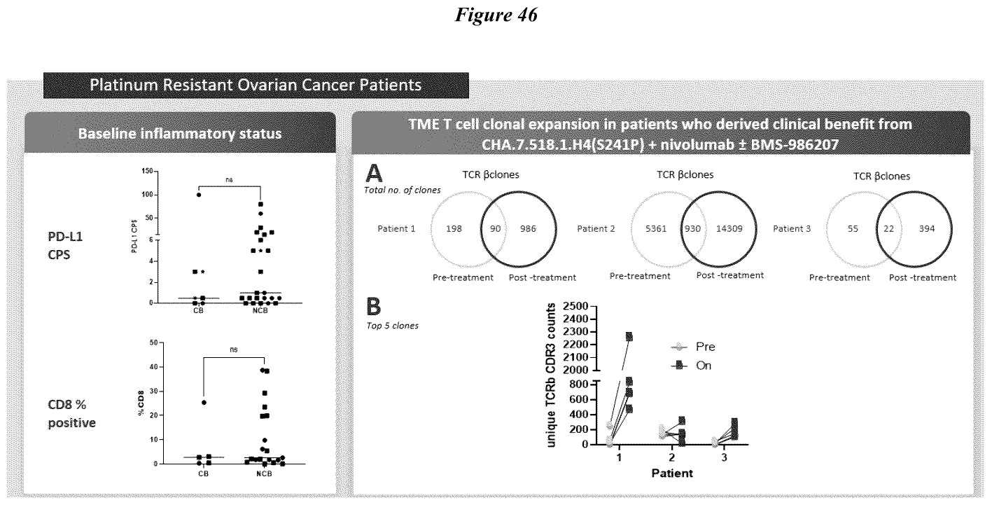

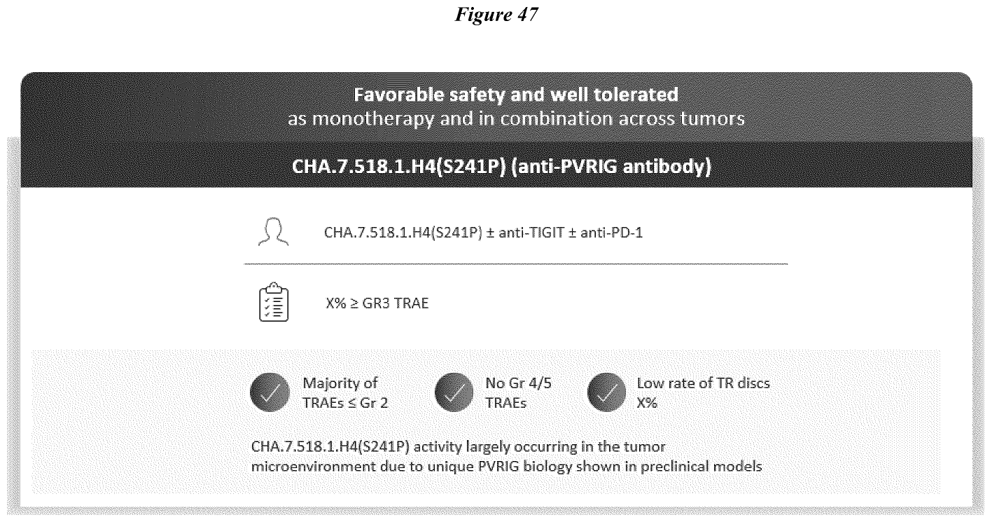

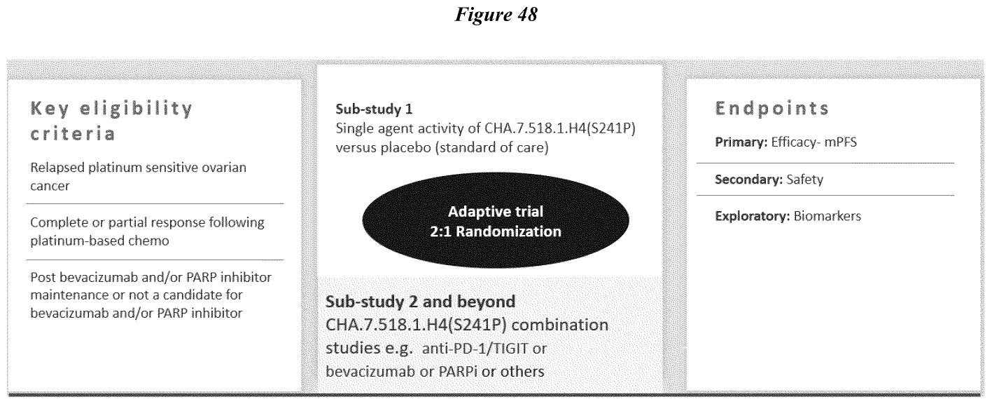

Data supporting the anti-tumor activity and safety profile of the triple combination CHA.7.518.1.H4(S241P) (ANTI-PVRIG), CPA.9.086.H4(S241P) (ANTI-TIGIT), and pembrolizumab in advanced heavily pre-treated patients with platinum resistant ovarian cancer (PROC) is provided herein. Also see, FIGS. 37-48.

The data from this study demonstrates that CHA.7.518.1.H4(S241P) is active, has a favorable safety profile, and is a differentiated immune checkpoint inhibitor. CHA.7.518.1.H4(S241P) in combination with CPA.9.086.H4(S241P) (Fc reduced anti-TIGIT) and pembrolizumab (anti-PD-1) resulted in durable objective responses in late-stage ovarian cancer patients typically not responsive to other immunotherapeutic agents. There is a significant unmet need for effective, durable, and tolerable treatment options for patients with relapsed ovarian cancer.

There is consistency of the data between the two platinum resistant ovarian cancer studies demonstrating CHA.7.518.1.H4(S241P) driven activity and safety profile in more than forty advanced and heavily pre-treated patients. These data support our initial observation of the unique mechanism of action of CHA.7.518.1.H4(S241P) translating into clinical benefit in patients with ovarian cancer. This feedback from ovarian cancer experts supports advancing CHA.7.518.1.H4(S241P) to an earlier setting of ovarian cancer therapy based on its overall activity, safety profile and durability demonstrated in advanced disease. There is a gap in care for women with platinum sensitive ovarian cancer, who respond to chemotherapy but are ineligible for or cannot tolerate additional maintenance treatment. These patients have a less compromised immune system, providing the opportunity to harness the unique mechanism of action of CHA.7.518.1.H4(S241P) to potentially change the disease trajectory improving progression free survival.

As noted, there is a gap in care for maintenance therapy in relapsed platinum sensitive ovarian cancer patients where safe and durable treatment options will have an advantage for women who have received prior maintenance treatment and have no options for additional maintenance treatment. This patient population is ranging between 8,000 to 12,000 women and is around one-third of the platinum sensitive 2L/3L patient population. Given the durability and tolerability profile of CHA.7.518.1.H4(S241P), CHA.7.518.1.H4(S241P) may fit this maintenance therapy need and has the potential to be highly differentiated in this setting and consequently may face less competition.

Moreover, these patients are less heavily pretreated and therefore have a less immune compromised system, providing the opportunity for CHA.7.518.1.H4(S241P) immunotherapy to harness its unique mechanism of action to potentially increase the time to disease progression and change the trajectory of the disease. In addition, platinum-based chemotherapy has been shown to induce tertiary lymphoid structures and T memory stem cells, and therefore has the potential to sensitize the tumors of the patients previously treated with chemotherapy to CHA.7.518.1.H4(S241P) by leveraging its unique mechanism of action on these cells.

Advancing CHA.7.518.1.H4(S241P) in this maintenance setting of platinum sensitive ovarian cancer has not only a strong clinical and biological rationale, it also takes into consideration the less competitive landscape and greater opportunity.

The trial is to be an adaptive platform trial, in relapsed platinum sensitive ovarian cancer patients who have received at least 2 prior lines of platinum-based chemotherapy regimens and are not candidates to receive standard of care maintenance treatment. Our development approach will be stepwise, starting with a randomized sub-study initially enrolling 60 patients, who will be randomized 2:1 to CHA.7.518.1.H4(S241P) monotherapy or to placebo. The primary endpoint will be median progression free survival, where the placebo benchmark is expected to be approximately six months.

Such a platform design allows for assessing CHA.7.518.1.H4(S241P) as a single-agent-maintenance therapy and opening additional arms (also referred to as sub-studies) in the future. Additional sub-studies would permit the evaluation of CHA.7.518.1.H4(S241P) as a backbone treatment, in combination with agents, like anti-PD-1/TIGIT checkpoint inhibitors, therefore supporting our DNAM-1 triplet combination strategy. Additional sub-studies would also permit exploring options like bevacizumab, PARP, ADCs or others, potentially with partners, to extract the full potential of CHA.7.518.1.H4(S241P) combinations. As part of the platform design, continued exploratory assessments of various potential biomarker enrichment strategies will be included.

The totality of the study data confirms that CHA.7.518.1.H4(S241P) is active and importantly that durable responses were reported with a good tolerability profile and the further development was justified as monotherapy and in combination, in platinum sensitive ovarian cancer, a setting which is particularly relevant for immunotherapy as the immune system is less compromised lending to an opportunity for CHA.7.518.1.H4(S241P) to change the course of the disease. In addition, the favorable safety profile of these drugs becomes an even greater point of differentiation in this earlier setting and as maintenance regimen. CHA.7.518.1.H4(S241P) alone and in combination was well tolerated, demonstrating consistent durable responses, in heavily pretreated PROC particularly in patients without liver metastases.

### Example 11: An Adaptive Clinical Platform Trial to Evaluate the Safety and Efficacy of CHA.7.518.1.H4(S241P) as Monotherapy or Combination Therapy as Maintenance Therapy in Participants with Relapsed Platinum Sensitive Ovarian Cancer (PSOC)

The goal of this clinical trial is to learn if the experimental antibody CHA.7.518.1.H4(S241P) delays the progression of ovarian cancer in participants with Relapsed Platinum Sensitive Ovarian Cancer. This study will also provide information regarding the safety of CHA.7.518.1.H4(S241P).

This study will address whether CHA.7.518.1.H4(S241P), when used as a maintenance treatment, will stop or slow the progression of ovarian cancer, and in particular in relapsed platinum sensitive ovarian cancer.

This study will also address whether CHA.7.518.1.H4(S241P) will delay the time to needing a new anti-cancer treatment.

This study will further address the side effects do participants have when taking CHA.7.518.1.H4(S241P).

Trial CPG-01-201 is an adaptive-platform trial comprised of multiple sub-studies.

Sub-study 1 will be a parallel group, double-blind, randomized placebo-controlled trial in which participants will be randomized in a 1:2 ratio to either placebo (a look-alike substance that contains no drug) or CHA.7.518.1.H4(S241P) treatment arms. This means that there is a 33% chance of being placed in the group getting placebo and a 67% chance of being placed in the group that will get the experimental CHA.7.518.1.H4(S241P) antibody.

Subsequent sub-studies will evaluate CHA.7.518.1.H4(S241P) in combination with other anticancer drugs.

### Parallel Assignment:

This study includes an adaptive platform trial enabling the evaluation of CHA.7.518.1.H4(S241P) initially as a single agent compared to placebo in relapsed PSOC. This will enable the evaluation of the effects of CHA.7.518.1.H4(S241P) as a single agent followed by the potential to adjust the trial to evaluate combinations with CHA.7.518.1.H4(S241P).

A data monitoring committee will review all safety and efficacy data on a regular basis and will make recommendations regarding study unblinding and timing to start additional sub-studies evaluating CHA.7.518.1.H4(S241P) in combination with other anticancer drugs.

An interim analysis will be conducted when approximately 60 participants in sub-study 1 have been followed for approximately 9 months.

### Arms and Interventions

| Arms | Assigned Interventions |
|---|---|
| Experimental: | Drug: CHA.7.518.1.H4(S241P) |
| CHA.7.518.1.H4(S241P) | CHA.7.518.1.H4(S241P) |
| | will be administered via |
| | intravenous (IV) infusion |
| | once every 3 weeks. |
| Placebo | Drug: Normal Saline |
| Comparator: | Saline will be administered |
| Normal Saline | via intravenous (IV) |
| | infusion once every 3 weeks |

### Outcome Measures

### Primary Outcome Measure:

To evaluate the effect of CHA.7.518.1.H4(S241P) as a single agent on progression free survival when administered as a maintenance regimen in participants with relapsed PSOC.
Progression free survival in CHA.7.518.1.H4(S241P)-treated participants compared to placebo-treated participants.
Time Frame: From the date of randomization until the date of disease progression, or date of death from any cause whichever occurs first, assessed up to 2 years.

### Secondary Outcome Measure:

To evaluate the safety and tolerability of the CHA.7.518.1.H4(S241P) as a single agent when administered as a maintenance regimen in participants with relapsed PSOC.
Number of participants with adverse events in the CHA.7.518.1.H4(S241P) treatment arm compared to the placebo treatment arm
Time Frame: From randomization up to 90 days post last cycle of study treatment (each cycle is 3 weeks; study treatment may continue up to 2 years).

To evaluate the effect of CHA.7.518.1.H4(S241P) as a single agent on other efficacy endpoints when administered as a maintenance regimen in participants with relapsed PSOC
Time to the initiation of a new anti-cancer treatment in CHA.7.518.1.H4(S241P)-treated participants compared to placebo-treated participants
Time Frame: From date of randomization until the date of the initiation of a new anti-cancer treatment, or date of death from any cause whichever occurs first, assessed up to 2 years.

### Inclusion Criteria:

Has relapsed platinum sensitive epithelial ovarian cancer, platinum sensitive fallopian tube cancer or platinum sensitive primary peritoneal cancer.

Has completed at least 2 previous courses (i.e., lines) of platinum containing therapy.

For the last chemotherapy course prior to being randomized into the study, must have had a minimum of 4 cycles of a platinum containing regimen and achieved a partial or complete tumor response.

Has received prior maintenance therapy with bevacizumab or a PARP inhibitor if eligible and is not a candidate for, or has declined in writing, bevacizumab or PARP inhibitor therapy.

Have recovered from toxicities of prior chemotherapy or other therapy (to grade 1 or less, except for alopecia and neuropathy recovered to a ≤grade 2).

### Exclusion Criteria:

Has had 4 or more lines of cytotoxic chemotherapy in total.

Is being treated with immunosuppressive doses of systemic medications, such as steroids within 2 weeks before study drug administration.

Has had prior treatment with PD-1, PD-L1, anti-PVRIG, TIGIT or any other check point inhibitors.

Presence of radiographic or biopsy proven liver metastases at the beginning or completion of current line of platinum-based chemotherapy.

Drainage of ascites during last 2 cycles of last chemotherapy or any time after completion of the last chemotherapy regimen.

Bowel obstruction in the 6 weeks prior to randomization.

Have known active central nervous system metastases and/or carcinomatous meningitis/leptomeningeal carcinomatosis.

Has active hepatitis B virus (HBV) or hepatitis C virus (HCV), or subjects with human immunodeficiency virus (HIV).

Has active and clinically relevant bacterial, fungal, or viral infection that is not controlled or requires systemic antibiotics, antifungals, or antivirals, respectively.

Has received a live viral vaccine within 30 days of planned start of study treatment or requiring a live vaccine during the study.

Has a history of severe allergic, anaphylactic, or other hypersensitivity reactions to a human or humanized monoclonal antibody (mAb) or allergy to any excipients in the investigational products.

Has any serious or unstable concomitant systemic disorder.

Has any other condition that may increase the risk associated with study participation or may interfere with the interpretation of study results and, in the opinion of the investigator, would make the subject inappropriate for entry into the study.

Is currently participating in or have participated in a clinical study and received an investigational agent or used an investigational device within 4 weeks prior to the first dose of study treatment.

Is pregnant or breastfeeding or planning to become pregnant during the period of the study.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method of maintenance treatment for platinum sensitive ovarian cancer, platinum sensitive epithelial ovarian cancer, platinum sensitive high grade ovarian cancer, platinum sensitive fallopian tube cancer, platinum sensitive peritoneal cancer, or platinum sensitive primary peritoneal cancer, the method comprising administering an anti-PVRIG antibody to a subject in need thereof, wherein
the anti-PVRIG antibody comprises:
i) a heavy chain variable domain comprising the vhCDR1, vhCDR2, and vhCDR3 from the heavy chain variable domain of CHA.7.518.1.H4(S241P) (SEQ ID NO:4), and
ii) a light chain variable domain comprising the vlCDR1, vlCDR2, and vlCDR3 from the light chain variable domain of CHA.7.518.1.H4(S241P) (SEQ ID NO:9); or
i) a heavy chain variable domain comprising the vhCDR1, vhCDR2, and vhCDR3 from the heavy chain variable domain of CHA.7.538.1.2.H4(S241P) (SEQ ID NO:14), and
ii) a light chain variable domain comprising the vlCDR1, vlCDR2, and vlCDR3 from the light chain variable domain of CHA.7.538.1.2.H4(S241P) (SEQ ID NO:19).

2. The method of maintenance treatment according to claim 1, wherein the subject has been previously treated with a platinum treatment and a standard of care maintenance therapeutic treatment: or wherein the subject has been previously treated with a platinum treatment and is ineligible and/or cannot tolerate a standard of care maintenance therapeutic treatment.

3. (canceled)

4. The method of maintenance treatment according to claim 1, wherein the subject has not been previously treated with a platinum treatment for greater than or equal to about 6 months prior to relapse of the ovarian cancer.

5. The method of maintenance treatment according to claim 1, wherein the subject is responsive to the previous platinum treatment.

6. The method of maintenance treatment according to claim 2, wherein the prior maintenance therapeutic treatment comprises bevacizumab and/or a PARP inhibitor.

7. (canceled)

8. The method of maintenance treatment according to claim 1, wherein the maintenance treatment further comprises administering to the subject in need thereof bevacizumab and/or a PARP inhibitor.

9. The method of maintenance treatment according to claim 1, wherein the anti-PVRIG antibody is administered as a stable liquid pharmaceutical formulation and, wherein the stable liquid pharmaceutical formulation of the anti-PVRIG antibody comprises:
a) from 10 mM to 100 mM histidine;
b) from 30 mM to 100 mM NaCl;
c) from 20 mM to 150 mM L-Arginine; and
d) from 0.005% to 0.1% w/v polysorbate 80,
wherein the formulation has a pH from 5.5 to 7.0.

10.-14. (canceled)

15. The method of maintenance treatment according to claim 1, wherein the pharmaceutical formulation comprises from 10 mM to 80 mM histidine, from 15 mM to 70 mM histidine, from 20 mM to 60 mM histidine, from 20 mM to 50 mM histidine, from 20 mM to 30 mM histidine, or about 25 mM histidine.

16. (canceled)

17. The method of maintenance treatment according to claim 1, wherein the pharmaceutical formulation comprises from 30 mM to 100 mM NaCl, from 30 mM to 90 mM NaCl, from 40 mM to 80 mM NaCl, from 30 mM to 70 mM histidine, from 45 mM to 70 mM NaCl, or about 60 mM NaCl.

18. (canceled)

19. The method of maintenance treatment according to claim 1, wherein the pharmaceutical formulation comprises from 20 mM to 140 mM L-arginine, from 30 mM to 140 mM L-arginine, from 40 mM to 130 mM L-arginine, from 50 mM to 120 mM L-arginine, from 60 mM to 110 mM L-arginine, from 70 mM to 110 mM L-arginine, from 80 mM to 110 mM L-arginine, from 90 mM to 110 mM L-arginine, or about 100 mM L-arginine.

20. (canceled)

21. The method of maintenance treatment according to claim 1, wherein the pharmaceutical formulation comprises from 0.006% to 0.1% w/v polysorbate 80, from 0.007% to 0.09% w/v polysorbate 80, from 0.008% to 0.08% w/v polysorbate 80, from 0.009% to 0.09% w/v polysorbate 80, from 0.01% to 0.08% w/v polysorbate 80, from 0.01% to 0.07% w/v polysorbate 80, from 0.01% to 0.07% w/v polysorbate 80, or from 0.01% to 0.06% w/v polysorbate 80, from 0.009% to 0.05% w/v polysorbate 80, or about 0.01% polysorbate 80.

22. (canceled)

23. The method of maintenance treatment according to claim 1, wherein the pH is from 6 to 7.0, from 6.3 to 6.8, or 6.5+/−0.2.

24.-25. (canceled)

26. The method of maintenance treatment according to claim 1, wherein the anti-PVRIG antibody is at a concentration of from 10 mg/mL to 40 mg/mL, 15 mg/mL to 40 mg/mL, 15 mg/mL to 30 mg/mL, 10 mg/mL to 25 mg/mL, or 15 mg/mL to 25 mg/mL.

27. The method of maintenance treatment according to claim 1, wherein said formulation is stable at −20° C. for at least 1 month, 3 months, 6 months, 9 months, 12 months, 18 months, 24 months, 30 months, 36 months, 42 months or 48 months: or at 2° C. to 8° C. for at least 1 week, 2 weeks, 3 weeks, 4 weeks, 5 weeks, 6 weeks, 7 weeks, 8 weeks, 9 weeks, 10 weeks, 1 month, 3 months, 6 months, 9 months, 12 months, 18 months, 24 months, 30 months, 36 months, 42 months or 48 months: or at about 20° C. to 25° C. for at least 1 week, 2 weeks, 3 weeks, 4 weeks, 5 weeks, 6 weeks, 1 months, 3 months, 6 months, or 9 months, 12 months, 18 months, or 36 months: or at 35° C. to 40° C. for at least 1 week, 2 weeks, 3 weeks, 4 weeks, 5 weeks, 6 weeks, 7 weeks, or 8 weeks.

28.-37. (canceled)

38. The method of maintenance treatment according to claim 1, wherein the anti-PVRIG antibody is administered every 1 week, 2 weeks, 3 weeks, or 4 weeks.

39. (canceled)

40. The method of maintenance treatment according to claim 1, wherein the anti-PVRIG antibody is administered at a dosage of about 0.01 mg/kg to about 20 mg/kg of the anti-PVRIG antibody, about 0.01 mg/kg to about 15 mg/kg of the anti-PVRIG antibody or about 0.01 mg/kg to about 10 mg/kg of the anti-PVRIG antibody, about 15 mg/kg, or about 20 mg/kg.

41.-42. (canceled)

43. The method of maintenance treatment according to claim 1, wherein the anti-PVRIG antibody is administered at a concentration of about 15 mg/kg or 20 mg/kg every 3 weeks.

44.-57. (canceled)

58. A method of treatment for cancer in a subject refractory or resistant to at least two or more prior therapeutic treatments, the method comprising administering an anti-TIGIT antibody, and anti-PVRIG antibody, and pembrolizumab, wherein:
a) the anti-PVRIG antibody comprises:
i) a heavy chain variable domain comprising the vhCDR1, vhCDR2, and vhCDR3 from the heavy chain variable domain of CHA.7.518.1.H4(S241P) (SEQ ID NO:4), and
ii) a light chain variable domain comprising the vlCDR1, vlCDR2, and vlCDR3 from the light chain variable domain of CHA.7.518.1.H4(S241P) (SEQ ID NO:9); or
iii) a heavy chain variable domain comprising the vhCDR1, vhCDR2, and vhCDR3 from the heavy chain variable domain of CHA.7.538.1.2.H4(S241P) (SEQ ID NO:14), and
iv) a light chain variable domain comprising the vlCDR1, vlCDR2, and vlCDR3 from the light chain variable domain of CHA.7.538.1.2.H4(S241P) (SEQ ID NO:19); and
b) the anti-TIGIT antibody comprises:
i) a heavy chain variable domain comprising the vhCDR1, vhCDR2, and vhCDR3 from the heavy chain variable domain of CPA.9.083.H4(S241P) (SEQ ID NO:24), and
ii) a light chain variable domain comprising the vlCDR1, vlCDR2, and vlCDR3 from the light chain variable domain of CPA.9.083.H4(S241P) (SEQ ID NO:29); or
i) a heavy chain variable domain comprising the vhCDR1, vhCDR2, and vhCDR3 from the heavy chain variable domain of CPA.9.086.H4(S241P) (SEQ ID NO:34), and
ii) a light chain variable domain comprising the vlCDR1, vlCDR2, and vlCDR3 from the light chain of CPA.9.086.H4(S241P) (SEQ ID NO:39).

59. The method according to claim 58, wherein the anti-PVRIG antibody is administered as a stable liquid pharmaceutical formulation and, wherein the stable liquid pharmaceutical formulation of the anti-PVRIG antibody comprises:
a) from 10 mM to 100 mM histidine;
b) from 30 mM to 100 mM NaCl;
c) from 20 mM to 150 mM L-Arginine; and
d) from 0.005% to 0.1% w/v polysorbate 80,
wherein the formulation has a pH from 5.5 to 7.0; and
wherein the cancer is selected from the group consisting of prostate cancer, liver cancer (HCC), rectal cancer, colorectal cancer (CRC), colorectal cancer MSS (MSS-CRC: microsatellite stable colorectal carcinoma/carcinoma), CRC (MSS unknown), metastatic MSS-CRC, refractory metastatic MSS-CRC, MSS-CRC with liver metastasis, ovarian cancer (including ovarian carcinoma), primary peritoneal ovarian carcinoma, advanced epithelial ovarian cancer, advanced epithelial ovarian carcinoma, platinum resistant ovarian cancer, platinum sensitive epithelial ovarian cancer, platinum sensitive high grade ovarian cancer, platinum sensitive fallopian tube cancer, platinum sensitive peritoneal cancer, platinum sensitive primary peritoneal cancer, high grade serous adenocarcinoma, endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), uveal melanoma, glioma, renal cell cancer (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cell cancer, MSI-high cancer, KRAS mutant tumors, adult T-cell leukemia/lymphoma, pleural mesothelioma, anal SCC, neuroendocrine lung cancer (including neuroendocrine lung carcinoma), small cell lung cancer, NSCLC, NSCLC large cell, NSCLC squamous cell, NSCLC adenocarcinoma, atypical carcinoid lung cancer, NSCLC with PDL1 >=50% TPS, cervical SCC, pancreatic cancer, pancreatic adenocarcinoma, adenoid cystic cancer (including adenoid cystic carcinoma), primary peritoneal cancer, microsatellite stable primary peritoneal cancer, platinum resistant microsatellite stable primary peritoneal cancer, Myelodysplastic syndromes (MDS), HNSCC, PD1 refractory or relapsing cancer, gastroesophageal junction cancer, gastric cancer, chordoma, sarcoma, endometrial sarcoma, chondrosarcoma, uterine sarcoma, plasma cell disorders, multiple myeloma, amyloidosis, AL-amyloidosis, glioblastoma, astrocytoma and fallopian tube cancer.

60.-158. (canceled)

159. A method for determining a cancer patient population for treatment with an anti-PVRIG antibody, an anti-TIGIT antibody, and an anti-PD-1 antibody, the method comprising:
a) detecting the presence of IL-6 in the serum from the cancer patient,
b) quantitating the measurement of the level of IL-6;
treating the cancer patient with an anti-PVRIG antibody, an anti-TIGIT antibody, and an anti-PD-1 antibody when IL-6 is present at a decreased and/or lower baseline serum level as compared to a control or a patient that is not treated with the anti-PVRIG antibody, and the anti-TIGIT antibody, and the anti-PD-1 antibody or relative to a cancer patient with a higher serum level of IL-6.

160.-192. (canceled)
